(19) United States
(12) Patent Application Publication
(10) Pub. No.: US 2013/0280170 A1
(43) Pub. Date: Oct. 24, 2013

(54) Imaging methods for oncolytic virus therapy

(71) Applicant: Aladar A. Szalay, Highland, CA (US)

(72) Inventor: Aladar A. Szalay, Highland, CA (US)

(21) Appl. No.: 13/815,728

(22) Filed: Mar. 13, 2013

Related U.S. Application Data
(60) Provisional application No. 61/687,347, filed on Apr. 20, 2012.

(51) Int. Cl. A61K 49/10; C12Q 1/70

(52) U.S. Cl. CPC A61K 49/10; C12Q 1/70
USPC 424/9.2; 435/5; 424/9.37

(57) ABSTRACT

Diagnostic methods for in vivo and ex vivo detection of oncolytic virus infection of tumors in a subject are provided. The diagnostic methods employ imaging agents that detect macrophage or inflammatory cells, such perfluorocarbon (PFC) imaging agents, to detection of inflammation associated with oncolytic virus administration, and, thereby detect tumors and cancers. Combinations and kits for use in the practicing the methods also are provided.

### PRIORITY CLAIM AND RELATED APPLICATIONS

Benefit of priority is claimed to U.S. Provisional Application Ser. No. 61/687,347, filed Apr. 20, 2012, to Aladar A. Szalay, entitled “IMAGING METHODS FOR ONCOLYTIC VIRUS THERAPY.”

This application is related to International PCT application No. Serial No. (Attorney Dkt. No. 33316-4839PC), filed Mar. 13, 2013, entitled “IMAGING METHODS FOR ONCOLYTIC VIRUS THERAPY,” which also claims priority to U.S. Provisional Application Ser. No. 61/687,347. The subject matter of each of the above-noted applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Mar. 11, 2013, is identical, 3.87 megabytes in size, and titled 4839seq.001.txt.

### FIELD OF INVENTION

Methods for detecting tumor colonization by an oncolytic virus and detecting and/or imaging tumors infected with an oncolytic virus are provided. Methods for determining the efficacy of an oncolytic virus therapy also are provided.

### BACKGROUND

Oncolytic viruses, such as vaccinia virus, have been shown to be effective for the diagnosis and therapy of tumors in preclinical models of a wide variety of cancers. Recent clinical studies have shown that these viruses also exhibit anti-tumor effects in humans. The effectiveness of oncolytic virus therapies depends on the localization and preferential accumulation of the viruses in the tumor following administration. Existing methods for detecting the presence of virus in a tumor depend on expression of a detectable protein encoded by the virus or the analysis of extracted tumor tissue. Detection of expressed proteins, such as fluorescent proteins or proteins that bind to contrast agents, can often be difficult to image in vivo and detection may depend on high levels of gene expression which may be toxic to the subject or difficult to attain. In addition, tumor biopsies for ex vivo detection are invasive procedures that can be painful, risky, and costly to the patient. Accordingly, there exists a need for non-invasive methods of detecting and assessing the level of tumor colonization by an oncolytic virus.

### SUMMARY

Provided are methods and combinations for detecting the accumulation of oncolytic virus in a subject to whom the virus is administered. The methods herein can be adapted for use with any method in which an oncolytic virus is administered. The methods detect accumulation of macrophage and/or other inflammatory cells that occur concomitant with oncolytic virus accumulation. The macrophage and/or other inflammatory cells are detected using reagents specific therefor. Detection of the macrophage and other inflammatory cells provides for detection of that viruses as they accumulate in the same loci that these cells occur. As a result, viral infection/accumulation can be monitored indirectly, and it is not necessary for the viruses encode or contain reporters or other detectable moieties, thus, permitting use of therapeutic oncolytic viruses that do not encode such extraneous products. Hence, for example, clonal strains and strains modified to encode therapeutic products only can be employed. The methods provided herein include any methods in which detection of viruses is required or is an element. Such methods include, but are not limited to, detection, diagnosis and monitoring of immunoprivileged cells, such as tumors, monitoring treatment thereof, including assessing efficacy of treatment by detecting, first infection of tumor cells and, then, their decrease. Efficacy assessment depends upon the stage of treatment. Initially virus accumulation is assessed, which is indicative that virus is colonizing a site and replicating, which is necessary for oncolysis. After such time, decreases in virus accumulation are indicative that treatment is effective. Thus, also provided are uses of agents for detection of macrophage and/or other inflammatory cells for detecting oncolytic viruses.

Any agent for detecting macrophage and/or other inflammatory cells can be employed in the methods provided herein. The macrophage and/or other inflammatory cells can be detected by imaging or by sampling body fluids or biopsies or other such methods. Exemplary of agents for detection of macrophage and/or other inflammatory cells are perfluorocarbons and perfluorocarbon-containing compounds. The agents typically are imaging agents for detection by imaging, such as MRI and PET method methods. The agents accumulate in the macrophages or cells by phagocytosis.

Thus, provided are methods for detecting and/or imaging cells and tissues infected with an oncolytic virus or detecting and/or imaging sites in which virus accumulates by administering an oncolytic virus to a subject; administering an agent for detection of macrophage or inflammatory cells in a subject; detecting or imaging the accumulation of macrophage or inflammatory cells in a subject. Oncolytic viruses accumulate in immunoprivileged tissues, such as tumors and wounds or other inflamed tissues. The methods provided herein, thus, can detect or image tumors cells in a subject by detecting or imaging the accumulation of macrophage or other inflammatory cells that are recruited when cells are infected by oncolytic viruses. Detection or imaging changes in such accumulation can detect or diagnose tumors, and/or monitor oncolytic virus therapy. The method of claim 1, wherein Oncolytic therapy can be monitored, for example, by imaging the macrophage to detect or image any changes in the profile of accumulated macrophage.

Since macrophage and/or inflammatory cells are distinct from the virus, the agents that detect them are administered separately from the virus. As a result, it is not necessary for the virus to encode any heterologous or otherwise detectable protein or protein that induces or produces a detectable signal. The viruses, however, can contain such protein as a separate or complementary way of detecting the virus. Thus, the oncolytic viruses can be wild type viruses or viruses that do not encode any heterologous gene products. The viruses, however, can encode heterologous gene products or heterologous nucleic acids, such as therapeutic proteins, including anti-tumor proteins. Thus, the virus can include heterologous nucleic acid that encodes a therapeutic or diagnostic reagent, such as, for example, an anticancer agent, an anti-metastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind to a contrasting agent, genes for optical imaging or detection, genes for PET imaging and genes for MRI imaging. Monitoring accumulation of viruses can be used to assess whether the therapy is effective.

Thus, among the methods provided herein are methods for assessing oncolytic virus infection of a tumor, by administering an oncolytic virus to a subject having a tumor; administering an agent for imaging macrophage or inflammatory cells to the subject; and detecting or imaging the accumulation of the agent in the tumor, wherein accumulation of the agent in the tumor is indicative that oncolytic virus has infected the tumor

Also provided are methods for r assessing the responsiveness of a tumor to oncolytic therapy by administering an oncolytic virus to a subject having a tumor; administering an agent for imaging macrophage or inflammatory cells to the subject; and detecting or imaging the accumulation of the agent, such as perfluorocarbon in the tumor. Accumulation of the agent in the tumor is indicative that the tumor is responsive to treatment with an oncolytic virus, thus, indicating that treatment is efficacious or that a particular virus is a candidate for therapy. Also detection of the accumulation of the macrophage or inflammatory cells can be used to detect or image or diagnose tumors. The agent can be detected or imaged in vivo in the subject or ex vivo in body fluid or tissue samples, such as biopsies. The agent can be detected or imaged by any suitable method, such as magnetic resonance imaging (MRI) or magnetic resonance spectroscopy (MRS).

The agent can be administered with the virus, sequentially, intermittently, in any order, in the same or a separate composition. For example, the imaging agent can be administered at a predetermined time prior to or after the administration of the oncolytic virus, such as at least 15 minutes, 30 minutes, 1 hour, 2 hours, 3 hours, 4 hours, 6 hours, 12 hours, 18 hours, 24 hours, or 48 hours or more prior to the administration of the oncolytic virus. The particular time can be empirically determined. The agent can be administered at a predetermined time following the administration of the oncolytic virus, such as, but not limited to, at least 1 hour, 2 hours, 3 hours, 4 hours, 6 hours, 12 hours, 18 hours, 24 hours, 2 days, 3 days, 4 days, 5 days, 6 days, 7 days, 8 days, 9 days, 10 days, 11 days, 12 days, 13 days, 14 days, 3 weeks 4 weeks, 1 month following the administration of the oncolytic virus. The agent can be detected or imaged, for example, at least 1 hour, 2 hours, 4 hours, 6 hours, 12 hours, 18 hours, 24 hours, 30 hours, 36 hours, 42 hours, or 48 hours following the administration of the perfluorocarbon imaging agent. The agent can be detected or imaged a plurality of times at successive time points following administration of the oncolytic virus or intermittently with administration of the virus or sequentially. The oncolytic virus can be administered in an amount sufficient to induce accumulation of the agent but that is lower than a treatment dosage of the virus, particularly for methods of diagnosis or detection of tumors, or for selecting or identify viruses that are candidates for treatment or for assessing efficacy before initiating treatment. Dosages of oncolytic virus administered can be empirically determined if needed, and include, but are not limited to, for example, 1×10² pfu to 1×10⁸ pfu, such as at least or at least about or is or is about 1×10² pfu, 1×10³ pfu, 1×10⁴ pfu, 1×10⁵ pfu, 1×10⁶ pfu, 1×10⁷ pfu or 1×10⁸ pfu. The viruses can be administered in therapeutic dosages, such as, but not limited to 1×10⁶ pfu to 1×10¹⁴ pfu, or an amount that is at least or at least about or is or is about 1×10⁶ pfu, 1×10⁷ pfu or 1×10⁸ pfu, 1×10⁹ pfu, 1×10¹⁰ pfu, 1×10¹¹ pfu, 1×10¹² pfu, 1×10¹³ pfu, or 1×10¹⁴ pfu. If a virus is tested for efficacy and is determined to accumulate in targeted tissues, treatment can continued, typically at a therapeutic dosage. For example, the virus can be administered in an amount that is at least 1×10⁹ pfu at least one time over at least one cycle of administration. It can be administered a plurality of times. The agent can be detected or imaged at a predetermined time(s) after each successive administration of the virus in a cycle of administration. The virus can be administered a single time or for a plurality of cycles. A cycle of administration can be, for example, at least or is two days, three days, four days, five days, six days, seven days, 14 days, 21 days or 28 days. The virus can be administered once or more, such as, two times, three times, four times, five times, six times or seven times over a cycle of administration. Exemplary of such protocols is administering the virus on the first day of a cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle.

The virus can be administered systemically or locally. It can be administered, for example, by intravenous, intraarterial, intratumoral, endoscopic, intralesional, intramuscular, intradermal, intraperitoneal, intravesicular, intraarticular, intrapleural, percutaneous, subcutaneous, oral, parenteral, intranasal, intratracheal, inhalation, intracranial, intraprostatic, intravitreal, topical, ocular, vaginal, rectal, transdermal and other such routes of administration.

The agents for detection or imaging include perfluorocarbon imaging agents. These include, for example, linear, branched and cyclic perfluorocarbons. Exemplary of perfluorocarbon imaging agents are those that contain a perfluorocarbon include perfluoroalkyl ethers, such as a perfluoropolyether, perfluoro crown ether, such as perfluoro-15-crown-5-ether, perfluoroalkane, perfluoropentane, perfluorohexane, perfluorononane, perfluorohexyl bromide, perfluorooctyl bromide, perfluorooctane, perfluorodecalin, perfluorocycloalkane, perfluoro amine, and mixtures thereof. The imaging agent can include a mixture of two or more perfluorocarbons.

The agents include perfluorocarbon imaging agents that contain a poly(ethylene oxide) block copolymer, such as for example, a poly(ethylene oxide)-polyester block copolymer, such as, but not limited to, poly(ethylene oxide)-block-poly(ε-caprolactone) copolymer, poly(ethylene oxide)-block-(L) polylactide copolymer, poly(ethylene oxide)-block-(D) polylactide copolymer, poly(ethylene oxide)-block-(D,L) polylactide copolymer, and combinations thereof. Exemplary of such is a poly(ethylene oxide) block copolymer is a poly(ethylene oxide)-polyether block copolymer, such as a polyethylene-polyether tri-block copolymer, including, for example, poly(ethylene oxide)-poly(propylene oxide)-poly(ethylene oxide) (PEO-PPO-PEO) tri-block copolymer.

The agents, particularly non-polar agents, can be formulated as an emulsion. For example, the agent can be a perfluorocarbon imaging agent, and can be present in the emulsion in an amount of, for example, from about 5% v/v to about 60% v/v, inclusive, of the emulsion, such as from about 10% v/v to about 40% v/v of the emulsion. The emulsions also can include other components, such as emulsification agents and an emulsion stabilizing agents.

The emulsions can contain nanoparticles with an average size of is less than or about 800 nm, such as, for example, less than or about 750 nm, less than or about 700 nm, less than or about 650 nm, less than or about 600 nm, less than or about 550 nm, less than or about 500 nm, less than or about 450 nm, than or about 400 nm, than or about 350 nm, than or about 300 nm, than or about 250 nm, than or about 200 nm, than or about 150 nm, than or about 100 nm, than or about 50 nm, or than or about 25 nm. The emulsion can have a polydispersity index ranging from about 0.1 to about 0.2.

The agent can contains a targeting moiety or a detectable moiety, such as a dye, a fluorescent molecule or a radio-label, such as an ¹⁸F isotope, for imaging, such as by positron emission tomography (PET) imaging

The methods can be practiced on mammalian subjects, including human and a non-human animal. Non-human animals include, but are not limited to, domesticated animals, zoo animals and farm animals, such as, for example an ape, monkey, gorilla, mouse, rat, rabbit, ferret, chicken, goat, cow, deer, sheep, horse, pig, dog or cat.

Oncolytic therapy can be combined with other therapies, including surgery, immunotherapy, radiation and/or chemotherapy. The agents can be used to monitor the effectiveness or assess the combination therapies. The tumors that can be detected and/or that are treated with the oncolytic virus include, but are not limited to, a tumor of the lung, breast, colon, brain, prostate, liver, pancreas, esophagus, kidney, stomach, thyroid, bladder, uterus, cervix or ovary. Tumors that can be detected include, but are not limited to, solid tumors, circulating tumors or cells, blood tumors and lymphatic tumors, metastatic tumors.

Many oncolytic therapeutic viruses are known. These include oncolytic herpes viruses, vesicular stomatitis viruses, reoviruses and vaccinia viruses, including NYAC, Lister strains, such as LIVP, Wyeth, WR and other strains. Exemplary of the viruses are LIVP strains, which include any known to those of skill in the art or developed or derived therefrom. Included for example, is an LIVP virus or modified form thereof designated GLV-1h68 (GLV-ONC1), viruses whose genome contains a sequence of nucleotides set forth in SEQ ID NO:2, or a sequence of nucleotides that has at least 90% or 95% sequence identity to SEQ ID NO:2 or a virus or modified form thereof that includes a genome that contains sequence of nucleotides set forth in SEQ ID NOS: 20-26, a sequence of nucleotides that has at least 97% sequence identity to a sequence of nucleotides set forth in SEQ ID NO: 20-26 or oncolytic virus that includes a sequence of nucleotides selected from a virus whose genome comprises SEQ ID NO:1, or a sequence of nucleotides that exhibits at least 99% sequence identity to any of SEQ ID NOS: 1.

Also included are clonal strains of viruses, such as clonal strains of LIVP and a modified form thereof. Included are LIVP that contain a sequence of nucleotides selected from: a) nucleotides 2,256-181,114 or 10,073-180,095 of SEQ ID NO:20, nucleotides 11,243-182,721 of SEQ ID NO:21, nucleotides 6,264-181,390 of SEQ ID NO:22, nucleotides 7,044-181,820 of SEQ ID NO:23, nucleotides 6,674-181,409 of SEQ ID NO:24, nucleotides 6,716-181,367 of SEQ ID NO:25 or nucleotides 6,899-181,870 of SEQ ID NO:26; and b) a sequence of nucleotides that has at least 97% sequence identity to a sequence of nucleotides 2,256-181,114 or 10,073-180,095 of SEQ ID NO:20, nucleotides 11,243-182,721 of SEQ ID NO:21, nucleotides 6,264-181,390 of SEQ ID NO:22, nucleotides 7,044-181,820 of SEQ ID NO:23, nucleotides 6,674-181,409 of SEQ ID NO:24, nucleotides 6,716-181,367 of SEQ ID NO:25 or nucleotides 6,899-181,870 of SEQ ID. The viruses can encode a heterologous gene product, such as a heterologous gene product is inserted into or in place of a non-essential gene or region in the genome of the virus. Exemplary are vaccinia viruses, such as LIVP viruses in which the heterologous gene product is inserted at the hemagglutinin (HA), thymidine kinase (TK), F14.5L, vaccinia growth factor (VGF), A35R, N1L, E2L/E3L, K1L/K2L, superoxide dismutase locus, 7.5K, C7-K1L, B13R+B14R, A26L or 14L gene loci in the genome of the virus. The nucleic acid encoding the heterologous gene product can be operatively linked to a promoter. The promoter can be a constitutive or inducible promoter. The heterologous nucleic acid can encode, for example, a therapeutic product and/or a reporter gene product, such as a fluorescent protein, a bioluminescent protein, a receptor or an enzyme. Exemplary fluorescent proteins include a green fluorescent protein, an enhanced green fluorescent protein, a blue fluorescent protein, a cyan fluorescent protein, a yellow fluorescent protein, a red fluorescent protein, or a far-red fluorescent protein, such as the Katushka (TurboFP635; available from Evrogen; see, also, Shcerobo et al. (2007) Nat. Methods 4:741-746), which is well-known far-red mutant of the red fluorescent protein from sea anemone Entacmaea quadricolor. Other reporter gene product include, but are not limited to, enzymes, such as a luciferase, β-glucuronidase, β-galactosidase, chloramphenicol acetyl tranferase (CAT), alkaline phosphatase and horseradish peroxidase. Enzymes can be detected by reaction of the enzyme with a substrate. The reporter gene product, for example, can be a receptor that binds to a detectable moiety or a ligand attached to a detectable moiety, such as, for example, a radiolabel, a chromogen or a fluorescent moiety.

The viruses also additionally can be detected by a different method, including, detection in vivo in the subject or ex vivo in a tumor biopsy sample from the subject. Expression of a reporter gene product in a virus can be detected by a method selected from among flow cytometry, fluorescence microscopy, fluorescence spectroscopy, magnetic resonance spectroscopy, positron emission tomography and luminescence spectroscopy.

Also provided are combinations that contain an oncolytic virus; and an agent for imaging microphage. The virus and agent are any described above and below herein, including the perfluorocarbon imaging agent and the vaccinia viruses. The combinations can be packaged as a kit, optionally including instructions for use and/or other reagents and devices or materials for implementation of the methods.

### DETAILED DESCRIPTION

### Outline

A. DEFINITIONS
B. OVERVIEW
C. METHODS FOR IMAGING TUMOR INFLAMMATION INDUCED BY ONCOLYTIC VIRUSES
   1. Exemplary Methods for Imaging Tumor Inflammation
      a. Exemplary Subjects
   2. Perfluorocarbon Imaging Agents for Use in the Methods
      a. Perfluorocarbon Emulsions
         i. Exemplary Perfluorocarbons (PFCs)
         (1) Modified perfluorocarbons
         ii. Selection of Perfluorocarbons
      b. Emulsification and Emulsion Stabilization Agents
         i. Block copolymers
      c. Characteristics of Perfluorocarbon Imaging Agent Emulsions
      d. Addition of Therapeutic and Diagnostic Agents
      e. Preparation of Perfluorocarbon Imaging Agent Emulsions
   3. Detection and Imaging Methods
      a. In vivo Detection and Imaging
      b. Ex vivo Detection and Imaging
D. VIRUSES FOR USE IN THE METHODS
   1. Exemplary Oncolytic Viruses
      a. Poxviruses
         i. Vaccinia Viruses
         (1) Modified Vaccinia Viruses
         (2) Exemplary Modified Vaccinia Viruses
      b. Other Oncolytic Viruses
      c. Production and Preparation of Virus
         i. Methods for Generating Recombinant Virus
   2. Expression of Therapeutic and Reporter Gene Products
      a. Exemplary Reporter Gene Products
      b. Exemplary Therapeutic Gene Products
      c. Operable linkage to promoter
   3. Further Modifications of Oncolytic Viruses
E. APPLICATIONS OF THE METHOD
   1. Assessment and Modification of Treatment and Selection of Viruses and Subjects for Therapy
   2. Application of the methods to tumor therapies with other microorganisms
   3. Methods of tracking inflammation using cells labeled with PFC imaging agents
   4. Therapy of Tumors Using Ultrasonic Release of Therapeutic Agents
F. COMBINATIONS, KITS, AND ARTICLES OF MANUFACTURE
G. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the term “perfluorocarbon,” or PFC, is used interchangeably herein with the term “fluorocarbon” and refers to a compound which includes a carbon backbone which is substituted with one or more fluorine atoms. In some examples, the perfluorocarbon does not include any covalent bonds between carbon and hydrogen. Exemplary perfluorocarbons include perfluorinated monovalent aliphatic groups (including perfluoroalkyls, alkenyls, and alkynyls) and perfluorinated aryl groups (such as phenyl, pyridinyl, and the like), as well as divalent groups such as perfluoroalkylene, perfluoroalkenylene, perfluoroalkynylene, and perfluoroarylene (e.g., 1,4-phenylene). A perfluorocarbon compound such as a perfluoroalkyl can be prepared from a corresponding non-fluorinated moiety, e.g., a hydrocarbyl moiety, by perfluorination, e.g., according to known methods for perfluorination. Perfluorocarbons can be straight or branched-chain, or cyclic. As used herein, the term “perfluorocarbon” encompasses perfluorocarbon derivatives that contain atoms other that carbon and fluorine. Perfluorocarbons include modified perfluorocarbons that contain additional moieties, such as for example, detectable moieties, such as a fluorescent dye or quantum nanodot particle, or targeting moieties.

As used herein, a perfluorocarbon imaging agent or PFC imaging agent, is a composition containing a perfluorocarbon (PFC) that can be detected by a magnetic resonance technique, such as, for example, magnetic resonance imaging (MRI) or magnetic resonance spectroscopy (MRS). Typically, the perfluorocarbon imaging agent is in the form of an emulsion that is used to contact cells in vitro or in vivo. The PFC emulsion typically contains PFCs, an emulsification agent and optionally other additives or surfactants.

As used herein, an emulsion is a composition containing a mixture of non-miscible components homogenously blended together. In particular examples, the non-miscible components include a lipophilic component and an aqueous component. An emulsion is a preparation of one liquid (e.g., the discrete or discontinuous phase) distributed in small nanoparticles throughout the body of a second liquid (e.g., the continuous phase). When oil is the dispersed liquid and an aqueous solution is the continuous phase, it is known as an oil-in-water emulsion. When water or an aqueous solution is the dispersed phase and oil or oleaginous substance is the continuous phase, it is known as a water-in-oil emulsion.

As used herein, a “nanoparticle” or “nano-sized” particle refers to a particle that is less than or equal to 500 nm in diameter.

As used herein, emulsifying/emulsification agent and emulsifier, are used interchangeably. The emulsifying agent are surface active substances which lower the interfacial tension of the liquids so that the non-oleaginous liquid may form a stable dispersion of fine droplets in the oleaginous liquid (i.e. an emulsion). Common emulsifiers include, but are not limited to metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxypropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In a particular example embodiment, the emulsifier is a poloxamer.

As used herein, an emulsion stabilization agent refers to an agent that acts to increase the stability of the emulsion and maintain the suspension of perfluorocarbon nanoparticles in the emulsion over time. Typically, emulsifying/emulsification agents and emulsifiers can also act as emulsion stabilization agents.

As used herein, a surfactant is a surface-active agent with a hydrophilic region and a hydrophobic region that lowers surface tension and thereby increases the emulsifying, foaming, dispersing, spreading and/or wetting properties of a substance. Surfactants include emulsifying/emulsification agents and emulsion stabilization agents and include non-ionic, cationic, anionic and zwitterionic agents.

As used herein, “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein the term “vaccinia virus” or “VACV” denotes a large, complex, enveloped virus belonging to the poxvirus family. It has a linear, double-stranded DNA genome approximately 190 kbp in length, and which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC 16MO, LIVP, WR 65-16, Connaught, New York City Board of Health vaccinia virus strains.

As used herein, Lister Strain of the Institute of Viral Preparations (LIVP) or LIVP virus strain refers to a virus strain that is the attenuated Lister strain (ATCC Catalog No. VR-1549) that was produced by adaption to calf skin at the Institute of Viral Preparations, Moscow, Russia (Al'tshtein et al. (1985) Dokl. Akad. Nauk USSR 285:696-699). The LIVP strain can be obtained, for example, from the Institute of Viral Preparations, Moscow, Russia (see. e.g., Kutinova et al. (1995) Vaccine 13:487-493); the Microorganism Collection of FSRI SRC VB Vector (Kozlova et al. (2010) Environ. Sci. Technol. 44:5121-5126); or can be obtained from the Moscow Ivanovsky Institute of Virology (C0355 K0602; Agranovski et al. (2006) Atmospheric Environment 40:3924-3929). It also is well known to those of skill in the art; it was the vaccine strain used for vaccination in the USSR and throughout Asia and India. The strain is used by researchers and is well known (see e.g., Altshteyn et al. (1985) Dokl. Akad. Nauk USSR 285:696-699; Kutinova et al. (1994) Arch. Virol. 134:1-9; Kutinova et al. (1995) Vaccine 13:487-493; Shchelkunov et al. (1993) Virus Research 28:273-283; Sroller et al. (1998) Archives Virology 143:1311-1320; Zinoviev et al. (1994) Gene 147:209-214; and Chkheidze et al. (1993) FEBS 336:340-342). Among the LIVP strains is one that contains a genome having a sequence of nucleotides set forth in SEQ ID NO: 2, or a sequence that is at least or at least about 99% identical to the sequence of nucleotides set forth in SEQ ID NO: 2.

As used herein, an LIVP clonal strain or LIVP clonal isolate refers to a virus that is derived from the LIVP virus strain by plaque isolation, or other method in which a single clone is propagated, and that has a genome that is homogenous in sequence. Hence, an LIVP clonal strain includes a virus whose genome is present in a virus preparation propagated from LIVP. An LIVP clonal strain does not include a recombinant LIVP virus that is genetically engineered by recombinant means using recombinant DNA methods to introduce heterologous nucleic acid. In particular, an LIVP clonal strain has a genome that does not contain heterologous nucleic acid that contains an open reading frame encoding a heterologous protein. For example, an LIVP clonal strain has a genome that does not contain non-viral heterologous nucleic acid that contains an open reading frame encoding a non-viral heterologous protein. As described herein, however, it is understood that any of the LIVP clonal strains provided herein can be modified in its genome by recombinant means to generate a recombinant virus. For example, an LIVP clonal strain can be modified to generate a recombinant LIVP virus that contains insertion of nucleotides that contain an open reading frame encoding a heterologous protein.

As used herein, the term “modified virus” refers to a virus that is altered compared to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one or more heterologous nucleic acid sequences in the form of a gene expression cassette for the expression of a heterologous gene.

As used herein, a modified LIVP virus strain refers to an LIVP virus that has a genome that is not contained in LIVP, but is a virus that is produced by modification of a genome of a strain derived from LIVP. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein. For example, modified viruses herein can contain one or more heterologous nucleic acid sequences in the form of a gene expression cassette for the expression of a heterologous gene.

As used herein, a subject includes any organism, including an animal, for whom diagnosis, screening, monitoring or treatment is contemplated. Animals include mammals, such as, for example, primates, domesticated animals and livestock. An exemplary primate is a human.

A patient refers to a subject, such as a mammal, primate, human, domesticated animal or livestock, or other animal subject afflicted with a disease condition or for which a disease condition is to be determined or risk of a disease condition is to be determined. Typically, a patient refers to a human subject exhibiting symptoms of a disease or disorder.

As used herein, animals include any animal, such as, but are not limited to, primates, including humans, apes and monkeys; rodents, such as mice, rats, rabbits, and ferrets; fowl, such as chickens; ruminants, such as goats, cows, deer, and sheep; horses, pigs, dogs, cats, fish, and other animals. Non-human animals exclude humans as the contemplated animal.

As used herein, the term “subject diagnosed with a cancer” refers to a subject who has been tested and found to have cancerous cells. The cancer can be diagnosed using any suitable method, including but not limited to, biopsy, x-ray, MRI, PET, blood test, and the diagnostic methods provided herein.

As used herein, a “tumor cell” is any cell that is part of a tumor or that is shed from a tumor (e.g. a circulating tumor cell). Tumor cells typically are cells undergoing early, intermediate, or advanced stages of neoplastic progression, including a pre-neoplastic cells (i.e. hyperplastic cells and dysplastic cells) and neoplastic cells.

As used herein, “tumorigenic cell,” is a cell that, when introduced into a suitable site in a subject, can form a tumor. The cell can be non-metastatic or metastatic.

As used herein, a “metastatic cell” is a cell that has the potential for metastasis. Metastatic cells have the ability to metastasize from a first tumor in a subject and can colonize tissue at a different site in the subject to form a second tumor at the site.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, a “normal cell” or “non-tumor cell” are used interchangeably and refer to a cell that is not derived from a tumor.

As used herein, the term “cell” refers to the basic unit of structure and function of a living organism as is commonly understood in the biological sciences. A cell can be a unicellular organism that is self-sufficient and that can exist as a functional whole independently of other cells. A cell also can be one that, when not isolated from the environment in which it occurs in nature, is part of a multicellular organism made up of more than one type of cell. Such a cell, which can be thought of as a “non-organism” or “non-organismal” cell, generally is specialized in that it performs only a subset of the functions performed by the multicellular organism as whole. Thus, this type of cell is not a unicellular organism. Such a cell can be a prokaryotic or eukaryotic cell, including animal cells, such as mammalian cells, human cells and non-human animal cells or non-human mammalian cells. Animal cells include any cell of animal origin that can be found in an animal. Thus, animal cells include, for example, cells that make up the various organs, tissues and systems of an animal.

As used herein an “isolated cell” is a cell that exists in vitro and is separate from the organism from which it was originally derived.

As used herein, a “cell line” is a population of cells derived from a primary cell that is capable of stable growth in vitro for many generations. Cell lines are commonly referred to as “immortalized” cell lines to describe their ability to continuously propagate in vitro.

As used herein a “tumor cell line” is a population of cells that is initially derived from a tumor. Such cells typically have undergone some change in vivo such that they theoretically have indefinite growth in culture; unlike primary cells, which can be cultured only for a finite period of time. Such cells can form tumors after they are injected into susceptible animals.

As used herein, a “primary cell” is a cell that has been isolated from a subject.

As used herein, a “host cell” or “target cell” are used interchangeably to mean a cell that can be infected by a virus.

As used herein, the term “tissue” refers to a group, collection or aggregate of similar cells generally acting to perform a specific function within an organism.

As used herein a “gene expression cassette” or “expression cassette” is a nucleic acid construct, containing nucleic acid elements that are capable of effecting expression of a gene in hosts that are compatible with such sequences. Expression cassettes include at least promoters and optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acid include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, a heterologous protein or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter also can be a hybrid promoter composed of different elements derived from different native promoters.

As used herein, a “reporter gene” is a gene that encodes a reporter molecule that can be detected when expressed by a virus provided herein or encodes a molecule that modulates expression of a detectable molecule, such as nucleic acid molecule or a protein, or modulates an activity or event that is detectable. Hence reporter molecules include, nucleic acid molecules, such as expressed RNA molecules, and proteins.

As used herein, a “heterologous reporter gene” is a reporter gene that is not natively present in a virus or is a gene that is present at a different locus than in its native locus in a virus. Heterologous reporter genes can contain nucleic acid that is not endogenous to the virus into which it is introduced, but has been obtained from another virus or cell or prepared synthetically. Heterologous reporter genes, however, can be endogenous, but contain nucleic acid that is expressed from a different locus or altered in its expression or sequence. Generally, such reporter genes encode RNA and proteins that are not normally produced by the virus or that are not produced under the same regulatory schema, such as the promoter.

As used herein, a “reporter protein” or “reporter gene product” refers to any detectable protein or product expressed by a reporter gene. Reporter proteins can be expressed from endogenous or heterologous genes. Exemplary reporter proteins are provided herein and include, for example, receptors or other proteins that can specifically bind to a detectable compound, proteins that can emit a detectable signal such as a fluorescence signal, and enzymes that can catalyze a detectable reaction or catalyze formation of a detectable product. Reporter gene products also can include detectable nucleic acids.

As used herein, a reporter virus is a virus that expresses or encodes a reporter gene or a reporter protein or a detectable protein or moiety. It is a virus that is detectable in a cell. As used herein, an oncolytic reporter virus is an oncolytic virus that expresses or encodes a reporter gene or a reporter protein or a detectable protein or moiety.

As used herein, detecting an oncolytic reporter virus means detecting tumor cells infected by the virus by one or more methods that detect a reporter gene product encoded by the virus that is expressed during infection of the tumor cell. Such methods include, but are not limited to detection of proteins such fluorescent proteins, luminescent proteins or proteins that bind to detectable ligands or antibodies.

As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak excitation spectrum at 490 nm or about 490 nm and peak emission spectrum at 510 nm or about 510 nm (expressed herein as excitation/emission 490 nm/510 nm). A variety of FPs that emit at various wavelengths are known in the art. Exemplary FPs include, but are not limited to, a violet fluorescent protein (VFP; peak excitation/emission at or about 355 nm/424 nm), a blue fluorescent protein (BFP; peak excitation/emission at or about 380-400 nm/450 nm), cyan fluorescent protein (CFP; peak excitation/emission at or about 430-460 nm/480-490 nm), green fluorescent protein (GFP; peak excitation/emission at or about 490 nm/510 nm), yellow fluorescent protein (YFP; peak excitation/emission at or about 515 nm/530 nm), orange fluorescent protein (OFP; peak excitation/emission at or about 550 nm/560 nm), red fluorescent protein (RFP; peak excitation/emission at or about 560-590 nm/580-610 nm), far-red fluorescent protein (peak excitation/emission at or about 590 nm/630-650 nm), or near-infrared fluorescent protein (peak excitation/emission at or about 690 nm/713 nm). Extending the spectrum of available colors of fluorescent proteins to blue, cyan, orange, yellow and red variants provides a method for multicolor tracking of proteins.

Examples of fluorescent proteins and their variants include, but are not limited to, GFPs, such as Emerald (EmGFP; Invitrogen, Carlsbad, Calif.), EGFP (Clontech, Palo Alto, Calif.), Azami-Green (MBL International, Woburn, Mass.), Kaede (MBL International, Woburn, Mass.), ZsGreen1 (Clontech, Palo Alto, Calif.) and CopGFP (Evrogen/Axxora, LLC, San Diego, Calif.); CFPs, such as Cerulean (Rizzo, Nat Biotechnol. 22(4):445-9 (2004)), mCFP (Wang et al. (2004) Proc. Natl. Acad. Sci. USA 101(48):16745-9), AmCyan1 (Clontech, Palo Alto, Calif.), MiCy (MBL International, Woburn, Mass.), and CyPet (Nguyen and Daugherty, Nat Biotechnol. 23(3):355-60 (2005)); BFPs, such as EBFP (Clontech, Palo Alto, Calif.); YFPs, such as EYFP (Clontech, Palo Alto, Calif.), YPet (Nguyen and Daugherty, Nat Biotechnol. 23(3):355-60 (2005)), Venus (Nagai et al. Nat. Biotechnol. 20(1):87-90 (2002)), ZsYellow (Clontech, Palo Alto, Calif.), and mCitrine (Wang et al., Proc. Natl. Acad. Sci. USA 101(48):16745-9 (2004)); OFPs, such as cOFP (Strategene, La Jolla, Calif.), mKO (MBL International, Woburn, Mass.), and mOrange; RFPs, such as Discosoma RFP (DsRed) isolated from the corallimorph Discosoma (Matz et al. (1999) Nature Biotechnology 17: 969-973) and Discosoma variants, such as monomeric red fluorescent protein 1 (mRFP1), mCherry, tdTomato, mStrawberry, mTangerine (Wang et al. (2004) Proc. Natl. Acad. Sci. USA 101(48):16745-9), DsRed2 (Clontech, Palo Alto, Calif.), and DsRed-T1 (Bevis and Glick, Nat. Biotechnol., 20: 83-87 (2002)), Anthomedusa J-Red (Evrogen) and Anemonia AsRed2 (Clontech, Palo Alto, Calif.); far-red FPs, such as Actinia AQ143 (Shkrob et al. (2005) Biochem J. 392(Pt 3):649-54), Entacmaea eqFP611 (Wiedenmann et al. (2002) Proc. Natl. Acad. Sci. USA. 99(18):11646-51), Discosoma variants, such as mPlum and mRasberry (Wang et al. (2004) Proc. Natl. Acad. Sci. USA 101(48):16745-9), Heteractis HcRed1 and t-HcRed (Clontech, Palo Alto, Calif.), TurboFP635 (Katushka), mKate, and mNeptune; near-infrared FPs, such as and IFP1.4 (Scherbo et al. (2007) Nat Methods 4:741-746), eqFP650 and eqFP670; and others (see, e.g., Shaner N C, Steinbach P A, and Tsien R Y. (2005) Nat Methods. 2(12):905-9 and Chudakov et al. (2010) Physil Rev 90:1103-1163 for description of additional exemplary FPs of various excitation/emission spectra)

As used herein, Aequorea GFP refers to GFPs from the genus Aequorea and to mutants or variants thereof. Such variants and GFPs from other species, such as Anthozoa reef coral, Anemonia sea anemone, Renilla sea pansy, Galaxea coral, Acropora brown coral, Trachyphyllia and Pectimidae stony coral and other species are well known and are available and known to those of skill in the art.

As used herein, luminescence refers to the detectable electromagnetic (EM) radiation, generally, ultraviolet (UV), infrared (IR) or visible EM radiation that is produced when the excited product of an exergonic chemical process reverts to its ground state with the emission of light. Chemiluminescence is luminescence that results from a chemical reaction. Bioluminescence is chemiluminescence that results from a chemical reaction using biological molecules (or synthetic versions or analogs thereof) as substrates and/or enzymes. Fluorescence is luminescence in which light of a visible color is emitted from a substance under stimulation or excitation by light or other forms radiation such as ultraviolet (UV), infrared (IR) or visible EM radiation.

As used herein, chemiluminescence refers to a chemical reaction in which energy is specifically channeled to a molecule causing it to become electronically excited and subsequently to release a photon, thereby emitting visible light. Temperature does not contribute to this channeled energy. Thus, chemiluminescence involves the direct conversion of chemical energy to light energy.

As used herein, bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which, upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives such as, for example, click beetle luciferase or firefly luciferase.

As used herein, luciferase refers to oxygenases that catalyze a light emitting reaction. For instance, bacterial luciferases catalyze the oxidation of flavin mononucleotide (FMN) and aliphatic aldehydes, which reaction produces light. Another class of luciferases, found among marine arthropods, catalyzes the oxidation of Cypridina (Vargula) luciferin and another class of luciferases catalyzes the oxidation of Coleoptera luciferin. Thus, luciferase refers to an enzyme or photoprotein that catalyzes a bioluminescent reaction (a reaction that produces bioluminescence). The luciferases, such as firefly and Gaussia and Renilla luciferases, are enzymes which act catalytically and are unchanged during the bioluminescence generating reaction. The luciferase photoproteins, such as the aequorin photoprotein to which luciferin is non-covalently bound, are changed, such as by release of the luciferin, during bioluminescence generating reaction. The luciferase is a protein, or a mixture of proteins (e.g., bacterial luciferase), that occurs naturally in an organism or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

Reference, for example, to Renilla luciferase refers to an enzyme isolated from member of the genus Renilla or an equivalent molecule obtained from any other source, such as from another related copepod, or that has been prepared synthetically. It is intended to encompass Renilla luciferases with conservative amino acid substitutions that do not substantially alter activity. Conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

As used herein, bioluminescence substrate refers to the compound that is oxidized in the presence of a luciferase and any necessary activators and generates light. These substrates are referred to as luciferins herein, are substrates that undergo oxidation in a bioluminescence reaction. These bioluminescence substrates include any luciferin or analog thereof or any synthetic compound with which a luciferase interacts to generate light. Typical substrates include those that are oxidized in the presence of a luciferase or protein in a light-generating reaction. Bioluminescence substrates, thus, include those compounds that those of skill in the art recognize as luciferins. Luciferins, for example, include firefly luciferin, Cypridina (also known as Vargula) luciferin (coelenterazine), bacterial luciferin, as well as synthetic analogs of these substrates or other compounds that are oxidized in the presence of a luciferase in a reaction the produces bioluminescence.

As used herein, capable of conversion into a bioluminescence substrate refers to being susceptible to chemical reaction, such as oxidation or reduction, which yields a bioluminescence substrate. For example, the luminescence producing reaction of bioluminescent bacteria involves the reduction of a flavin mononucleotide group (FMN) to reduced flavin mononucleotide (FMNH₂) by a flavin reductase enzyme. The reduced flavin mononucleotide (substrate) then reacts with oxygen (an activator) and bacterial luciferase to form an intermediate peroxy flavin that undergoes further reaction, in the presence of a long-chain aldehyde, to generate light. With respect to this reaction, the reduced flavin and the long chain aldehyde are bioluminescence substrates.

As used herein, a bioluminescence generating system refers to the set of reagents required to conduct a bioluminescent reaction. Thus, the specific luciferase, luciferin and other substrates, solvents and other reagents that can be required to complete a bioluminescent reaction form a bioluminescence system. Thus a bioluminescence generating system refers to any set of reagents that, under appropriate reaction conditions, yield bioluminescence. Appropriate reaction conditions refer to the conditions necessary for a bioluminescence reaction to occur, such as pH, salt concentrations and temperature. In general, bioluminescence systems include a bioluminescence substrate, luciferin, a luciferase, which includes enzymes luciferases and photoproteins and one or more activators. A specific bioluminescence system can be identified by reference to the specific organism from which the luciferase derives; for example, the Renilla bioluminescence system includes a Renilla luciferase, such as a luciferase isolated from Renilla or produced using recombinant methods or modifications of these luciferases. This system also includes the particular activators necessary to complete the bioluminescence reaction, such as oxygen and a substrate with which the luciferase reacts in the presence of the oxygen to produce light.

As used herein, the term “modified” with reference to a gene refers to a gene encoding a gene product, having one or more truncations, mutations, insertions or deletions; to a deleted gene; or to a gene encoding a gene product that is inserted (e.g., into the chromosome or on a plasmid, phagemid, cosmid, and phage), typically accompanied by at least a change in function of the modified gene product or virus.

As used herein, a “non-essential gene or region” of a virus genome is a location or region that can be modified by insertion, deletion, or mutation without inhibiting the infection life cycle of the virus. Modification of a “non-essential gene or region” is intended to encompass modifications that have no effect on the virus life cycle and modifications that attenuate or reduce the toxicity of the virus, but do not completely inhibit infection, replication and production of new virus.

As used herein, an “attenuated virus” refers to a virus that has been modified to alter one or more properties of the virus that affect, for example, virulence, toxicity, or pathogenicity of the virus compared to a virus without such modification. Typically, the viruses for use in the methods provided herein are attenuated viruses with respect to the wild-type form of the virus.

As used herein, an “attenuated LIVP virus” with reference to LIVP refers to a virus that exhibits reduced or less virulence, toxicity or pathogenicity compared to LIVP.

As used herein, “toxicity” (also referred to as virulence or pathogenicity herein) with reference to a virus refers to the deleterious or toxic effects to a host upon administration of the virus. For an oncolytic virus, such as LIVP, the toxicity of a virus is associated with its accumulation in non-tumorous organs or tissues, which can impact the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on weight.

As used herein, “reduced toxicity” means that the toxic or deleterious effects upon administration of the virus to a host are attenuated or lessened compared to a host that is administered with another reference or control virus. For purposes herein, exemplary of a reference or control virus with respect to toxicity is the LIVP virus designated GLV-1h68 (described, for example, in U.S. Pat. No. 7,588,767) or a virus that is the same as the virus administered except not including a particular modification that reduces toxicity. Whether toxicity is reduced or lessened can be determined by assessing the effect of a virus and, if necessary, a control or reference virus, on a parameter indicative of toxicity. It is understood that when comparing the activity of two or more different viruses, the amount of virus (e.g. pfu) used in an in vitro assay or administered in vivo is the same or similar and the conditions (e.g. in vivo dosage regime) of the in vitro assay or in vivo assessment are the same or similar. For example, when comparing effects upon in vivo administration of a virus and a control or reference virus the subjects are the same species, size, gender and the virus is administered in the same or similar amount under the same or similar dosage regime. In particular, a virus with reduced toxicity can mean that upon administration of the virus to a host, such as for the treatment of a disease, the virus does not accumulate in non-tumorous organs and tissues in the host to an extent that results in damage or harm to the host, or that impacts survival of the host to a greater extent than the disease being treated does or to a greater extent than a control or reference virus does. For example, a virus with reduced toxicity includes a virus that does not result in death of the subject over the course of treatment.

As used herein, accumulation of a virus in a particular tissue refers to the distribution of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s), the immunocompetence of the host and dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, “preferential accumulation” refers to accumulation of a virus at a first location at a higher level than accumulation at a second location (i.e., the concentration of viral particles, or titer, at the first location is higher than the concentration of viral particles at the second location). Thus, a virus that preferentially accumulates in immunoprivileged tissue (tissue that is sheltered from the immune system), such as inflamed tissue, and tumor tissue, relative to normal tissues or organs, refers to a virus that accumulates in immunoprivileged tissue, such as tumor, at a higher level (i.e., concentration or viral titer) than the virus accumulates in normal tissues or organs.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, “greater” or “improved” activity with reference to anti-tumor activity or anti-tumorigenicity means that a virus strain is capable of preventing or inhibiting the formation or growth of tumors in vitro or in vivo in a subject to a greater extent than a reference or control virus or to a greater extent than absence of treatment with the virus. Whether anti-tumor activity is “greater” or “improved” can be determined by assessing the effect of a virus and, if necessary, a control or reference virus, on a parameter indicative of anti-tumor activity. It is understood that when comparing the activity of two or more different viruses, the amount of virus (e.g. pfu) used in an in vitro assay or administered in vivo is the same or similar, and the conditions (e.g. in vivo dosage regime) of the in vitro assay or in vivo assessment are the same or similar.

As used herein, “genetic therapy” or “gene therapy” involves the transfer of heterologous nucleic acid, such as DNA, into certain cells, target cells, of a mammal, particularly a human, with a disorder or conditions for which such therapy is sought. The nucleic acid, such as DNA, is introduced into the selected target cells, such as directly or in a vector or other delivery vehicle, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy also can be used to deliver nucleic acid encoding a gene product that replaces a defective gene or supplements a gene product produced by the mammalian or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, the terms overproduce or overexpress when used in reference to a substance, molecule, compound or composition made in a cell refers to production or expression at a level that is greater than a baseline, normal or usual level of production or expression of the substance, molecule, compound or composition by the cell. A baseline, normal or usual level of production or expression includes no production/expression or limited, restricted or regulated production/expression. Such overproduction or overexpression is typically achieved by modification of cell.

As used herein, a tumor, also known as a neoplasm, is an abnormal mass of tissue that results when cells proliferate at an abnormally high rate. Tumors can show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and blastic tumors.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, proliferative disorders include any disorders involving abnormal proliferation of cells (i.e. cells proliferate more rapidly compared to normal tissue growth), such as, but not limited to, neoplastic diseases.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, an aggressive cancer refers to a cancer characterized by a rapidly growing tumor or tumors. Typically the tumor(s) is actively metastasizing or is at risk of metastasizing. Aggressive cancer typically refer to metastatic cancers that spread to multiple locations in the body.

As used herein, an in vivo method refers to any method that is performed within the living body of a subject. As used herein, an in vitro method refers to any method that is performed outside the living body of a subject.

As used herein, an ex vivo method refers to a method performed on a sample obtained from a subject.

As used herein, the term “therapeutic virus” refers to a virus that is administered for the treatment of a disease or disorder, such as a neoplastic disease, such as cancer, a tumor and/or a metastasis or inflammation or wound or diagnosis thereof and or both. Generally, a therapeutic virus herein is one that exhibits anti-tumor activity and minimal toxicity.

As used herein, a disease or disorder refers to a pathological condition in an organism resulting from, for example, infection or genetic defect, and characterized by identifiable symptoms.

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, efficacy means that upon systemic administration of an oncolytic virus, the virus will colonize tumor cells and replicate. In particular, it will replicate sufficiently so that tumor cells released into circulation will contain virus. Colonization and replication in tumor cells is indicative that the treatment is or will be an effective treatment.

As used herein, effective treatment with a virus is one that can increase survival compared to the absence of treatment therewith. For example, a virus is an effective treatment if it stabilizes disease, causes tumor regression, decreases severity of disease or slows down or reduces metastasizing of the tumor.

As used herein, therapeutic agents are agents that ameliorate the symptoms of a disease or disorder or ameliorate the disease or disorder. Therapeutic agents can be any molecule, such as a small molecule, a peptide, a polypeptide, a protein, an antibody, an antibody fragment, a DNA, or a RNA. Therapeutic agent, therapeutic compound, or therapeutic regimens include conventional drugs and drug therapies, including vaccines for treatment or prevention (i.e., reducing the risk of getting a particular disease or disorder), which are known to those skilled in the art and described elsewhere herein. Therapeutic agents for the treatment of neoplastic disease include, but are not limited to, moieties that inhibit cell growth or promote cell death, that can be activated to inhibit cell growth or promote cell death, or that activate another agent to inhibit cell growth or promote cell death. Therapeutic agents for use in the methods provided herein can be, for example, an anticancer agent. Exemplary therapeutic agents include, for example, therapeutic microorganisms, such as therapeutic viruses and bacteria, chemotherapeutic compounds, cytokines, growth factors, hormones, photosensitizing agents, radionuclides, toxins, antimetabolites, signaling modulators, anticancer antibiotics, anticancer antibodies, anti-cancer oligopeptides, anti-cancer oligonucleotide (e.g., antisense RNA and siRNA), angiogenesis inhibitors, radiation therapy, or a combination thereof.

As used herein, an anti-cancer agent or compound (used interchangeably with “anti-tumor or anti-neoplastic agent”) refers to any agents, or compounds, used in anti-cancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein.

As used herein, a “chemotherapeutic agent” is any drug or compound that is used in anti-cancer treatment. Exemplary of such agents are alkylating agents, nitrosoureas, antitumor antibiotics, antimetabolites, antimitotics, topoisomerase inhibitors, monoclonal antibodies, and signaling inhibitors. Exemplary chemotherapeutic agent include, but are not limited to, chemotherapeutic agents, such as Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. The term “chemotherapeutic agent” can be used interchangeably with the term “anti-cancer agent” when referring to drugs or compounds for the treatment of cancer. As used herein, reference to a chemotherapeutic agent includes combinations or a plurality of chemotherapeutic agents unless otherwise indicated.

As used herein, an anti-metastatic agent is an agent that ameliorates the symptoms of metastasis or ameliorates metastasis. Generally, anti-metastatic agents directly or indirectly inhibit one or more steps of metastasis, including but not limited to, degradation of the basement membrane and proximal extracellular matrix, which leads to tumor cell detachment from the primary tumor, tumor cell migration, tumor cell invasion of local tissue, tumor cell division and colonization at the secondary site, organization of endothelial cells into new functioning capillaries in a tumor, and the persistence of such functioning capillaries in a tumor. Anti-metastatic agents include agents that inhibit the metastasis of a cell from a primary tumor, including release of the cell from the primary tumor and establishment of a secondary tumor, or that inhibits further metastasis of a cell from a site of metastasis. Treatment of a tumor bearing subject with anti-metastatic agents can result in, for example, the delayed appearance of secondary (i.e. metastatic) tumors, slowed development of primary or secondary tumors, decreased occurrence of secondary tumors, slowed or decreased severity of secondary effects of neoplastic disease, arrested tumor growth and regression.

As used herein, an effective amount of a virus or compound for treating a particular disease is an amount that is sufficient to ameliorate, or in some manner reduce the symptoms associated with the disease. Such an amount can be administered as a single dosage or can be administered in multiple dosages according to a regimen, whereby it is effective. The amount can cure the disease but, typically, is administered in order to ameliorate the symptoms of the disease. Repeated administration can be required to achieve the desired amelioration of symptoms.

As used herein, a compound produced in a tumor refers to any compound that is produced in the tumor or tumor environment by virtue of the presence of an introduced virus, generally a recombinant virus, expressing one or more gene products. For example, a compound produced in a tumor can be, for example, an encoded polypeptide or RNA, a metabolite, or compound that is generated by a recombinant polypeptide and the cellular machinery of the tumor.

As used herein, the term “ELISA” refers to enzyme-linked immunosorbent assay. Numerous methods and applications for carrying out an ELISA are well known in the art, and provided in many sources (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]; and Newton, et al. (2006) Neoplasia. 8:772-780). A “direct ELISA” protocol involves a target-binding molecule, such as a cell, cell lysate, or isolated protein, first bound and immobilized to a microtiter plate well. A “sandwich ELISA” involves a target-binding molecule attached to the substrate by capturing it with an antibody that has been previously bound to the microtiter plate well. The ELISA method detects an immobilized ligand-receptor complex (binding) by use of fluorescent detection of fluorescently labeled ligands or an antibody-enzyme conjugate, where the antibody is specific for the antigen of interest, such as a phage virion, while the enzyme portion allows visualization and quantitation by the generation of a colored or fluorescent reaction product. The conjugated enzymes commonly used in the ELISA include horseradish peroxidase, urease, alkaline phosphatase, glucoamylase or O-galactosidase. The intensity of color development is proportional to the amount of antigen present in the reaction well.

As used herein, a delivery vehicle for administration refers to a lipid-based or other polymer-based composition, such as liposome, micelle or reverse micelle, that associates with an agent, such as a virus provided herein, for delivery into a host subject.

As used herein, a “diagnostic agent” refer to any agent that can be applied in the diagnosis or monitoring of a disease or health-related condition. The diagnostic agent can be any molecule, such as a small molecule, a peptide, a polypeptide, a protein, an antibody, an antibody fragment, a DNA, or a RNA.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured. Detectable labels can be used to image one or more of any of the viruses provided herein. Detectable labels can be used in any of the methods provided herein. Detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition. Exemplary detectable labels include, for example, metals such as colloidal gold, iron, gadolinium, and gallium-67, fluorescent moieties, and radionuclides. Exemplary fluorescent moieties and radionuclides are provided elsewhere herein.

As used herein, a radionuclide, a radioisotope or radioactive isotope is used interchangeably to refer to an atom with an unstable nucleus. The nucleus is characterized by excess energy which is available to be imparted either to a newly-created radiation particle within the nucleus, or else to an atomic electron. The radionuclide, in this process, undergoes radioactive decay, and emits a gamma ray and/or subatomic particles. Such emissions can be detected in vivo by method such as, but not limited to, positron emission tomography (PET), single-photon emission computed tomography (SPECT) or planar gamma imaging. Radioisotopes can occur naturally, but also can be artificially produced. Exemplary radionuclides for use in in vivo imaging include, but are not limited to, ¹¹C, ¹¹F, ¹³C, ¹³N, ¹⁵N, ¹⁵0, ¹⁸F, ¹⁹F, ³²P, ⁵³Fe, ⁵¹Cr, ⁵⁵Co, ⁵⁵Fe, ⁵⁷Co, ⁵⁸Co, ⁵⁷Ni, ⁵⁹Fe ⁶⁰Co, ⁶⁴Cu, ⁶⁷Ga, ⁶⁸Ga, ⁶⁰Cu(II), ⁶⁷Cu(II), ⁹⁹Tc, ⁹⁰Y, ⁹⁹Tc, ¹⁰³Pd, ¹⁰⁶Ru, ¹¹¹In, ¹¹⁷Lu, ¹²³I, ¹²⁵I, ¹²⁴I, ¹³¹I, ¹³⁷Cs, ¹⁵³Gd, ¹⁵³Sm, ¹⁸⁶Re, ¹⁸⁸Re, ¹⁹²Ir, ¹⁹⁸Au, ²¹¹At, ²¹²Bi, ²¹³Bi and ²⁴¹Am. Radioisotopes can be incorporated into or attached to a compound, such as a metabolic compound. Exemplary radionuclides that can be incorporated or linked to a metabolic compound, such as nucleoside analog, include, but are not limited to, ¹²³I, ¹²⁴I, ¹²⁵I, ¹³¹I, ¹⁸F, ¹⁹F, ¹¹C, ¹³C, ¹⁴C, ⁷⁵Br, and ³H.

As used herein, magnetic resonance imaging (MRI) refers to the use of a nuclear magnetic resonance spectrometer to produce electronic images of specific atoms and molecular structures in solids, especially human cells, tissues, and organs. MRI is non-invasive diagnostic technique that uses nuclear magnetic resonance to produce cross-sectional images of organs and other internal body structures. The subject lies inside a large, hollow cylinder containing a strong electromagnet, which causes the nuclei of certain atoms in the body (such as, for example, ¹H, ¹³C and ¹⁹F) to align magnetically. The subject is then subjected to radio waves, which cause the aligned nuclei to flip; when the radio waves are withdrawn the nuclei return to their original positions, emitting radio waves that are then detected by a receiver and translated into a two-dimensional picture by computer. For some MRI procedures, contrast agents such as gadolinium are used to increase the accuracy of the images.

As used herein, an X-ray refers to a relatively high-energy photon, or a stream of such photons, having a wavelength in the approximate range from 0.01 to 10 nanometers. X-rays also refer to photographs taken with x-rays.

As used herein, a compound conjugated to a moiety refers to a complex that includes a compound bound to a moiety, where the binding between the compound and the moiety can arise from one or more covalent bonds or non-covalent interactions such as hydrogen bonds, or electrostatic interactions. A conjugate also can include a linker that connects the compound to the moiety. Exemplary compounds include, but are not limited to, nanoparticles and siderophores. Exemplary moieties, include, but are not limited to, detectable moieties and therapeutic agents.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, an agent or compound that modulates the activity of a protein or expression of a gene or nucleic acid either decreases or increases or otherwise alters the activity of the protein or, in some manner, up- or down-regulates or otherwise alters expression of the nucleic acid in a cell.

As used herein, “nucleic acids” include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a sequence complementary to at least a portion of an RNA, with reference to antisense oligonucleotides, means a sequence of nucleotides having sufficient complementarity to be able to hybridize with the RNA, generally under moderate or high stringency conditions, forming a stable duplex; in the case of double-stranded antisense nucleic acids, a single strand of the duplex DNA (i.e., dsRNA) can thus be assayed, or triplex formation can be assayed. The ability to hybridize depends on the degree of complementarity and the length of the antisense nucleic acid. Generally, the longer the hybridizing nucleic acid, the more base mismatches with an encoding RNA it can contain and still form a stable duplex (or triplex, as the case can be). One skilled in the art can ascertain a tolerable degree of mismatch by use of standard procedures to determine the melting point of the hybridized complex.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Amino Acid Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§1.821-1.822, and incorporated herein by reference. Furthermore, a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carillo, H. & Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al. Nucleic Acids Research 12(I):387 (1984)), BLASTP, BLASTN, FASTA (Atschul, S. F., et al. J Mol Biol 215:403 (1990)); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result is independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. For example for β-glucuronidase, exemplary of species variants provided herein are mouse, rat, cat, dog, pig, green monkey and Sumatran orangutan. Generally, species variants have 70%, 75%, 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. An amino-acid substituted polypeptide can exhibit 65%, 70%, 80%, 85%, 90%, 91%, 92%, 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Methods of modifying a polypeptide are routine to those of skill in the art, such as by using recombinant DNA methodologies.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table 2 as follows:

TABLE 2
Table of Exemplary Conservative Amino Acid Substitutions

| Original residue | Exemplary Conservative Substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, refers to preparations of proteins that are substantially free of cellular material includes preparations of proteins in which the protein is separated from cellular components of the cells from which it is isolated or recombinantly-produced. In one example, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well known methods of molecular biology for expressing proteins encoded by cloned DNA.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, vector (or plasmid) refers to a nucleic acid construct that contains discrete elements that are used to introduce heterologous nucleic acid into cells for either expression of the nucleic acid or replication thereof. The vectors typically remain episomal, but can be designed to effect stable integration of a gene or portion thereof into a chromosome of the genome. Selection and use of such vectors are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “viral vector” is used according to its art-recognized meaning. It refers to a nucleic acid vector that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, poxvirus vectors (e.g., vaccinia vectors), retroviral vectors, lentivirus vectors, herpes virus vectors (e.g., HSV), baculovirus vectors, cytomegalovirus (CMV) vectors, papillomavirus vectors, simian virus (SV40) vectors, semliki forest virus vectors, phage vectors, adenoviral vectors and adeno-associated viral (AAV) vectors.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination, optionally, including instructions for use of the combination and/or other reactions and components for such use.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Hence, “about 5 milliliters” means “about 5 milliliters” and also “5 milliliters.” Generally “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. OVERVIEW

Oncolytic viruses effect treatment of tumors by infecting and accumulating in tumor cells. Studies have shown that administration of oncolytic viruses to tumor bearing subjects also induces the recruitment of inflammatory cells to the tumor, including macrophages, cytotoxic T cells and natural killer cells. For example, vaccinia virus infection of mouse xenograft models of breast cancer has been shown to result in massive recruitment of MHCII-positive leukocytes as determined by immunohistochemistry of excised tumors (Weibel et al. (2011) BMC Cancer 11:68). The influx of inflammatory cells was correlated with tumor cell infection by the virus and/or viral replication in the tumor as evidenced by tumor cell expression of green fluorescent protein (GFP) encoded by the virus. Similar recruitment of inflammatory cells to virus-infected tumors also has been shown for other oncolytic viruses, such as adenovirus, herpesviruses, and vesicular stomatitis virus (see, e.g. Martuza et al. (1991) Science 252(5007) 854-856, Fulci and Chiocca (2003) Frontiers in Bioscience 8:e346-e360, Markert et al. (2008) Molecular Therapy 17(1):199-207, Diaz et al. (2007) Cancer Research 67(6):2840-2848, Breitback et al. (2007) Molecular Therapy 15:9:1686-1693 and Woller et al. (2011) J Clin Invest. 121(7):2570-2582).

Perfluorocarbon (PFC) imaging agents, such as emulsions of perfluoropolyethers (PFPEs), have been used in a variety of different preclinical studies to detect inflammation in vivo and ex vivo in excised tissues (see, e.g. Flogel et al. (2008) Circulation 118(2):140-148, Weise et al. (2011) Experimental Neurology 229:494-501, Hitchens et al. (2011) Magnetic Resonance in Medicine 65:1145-1154, Ahrens et al. (2010) BioTechniques 50(4):229-234 and Hertlein et al. (2011) PLoS ONE 6(3):e18246). The PFC agents are taken by phagocytic cells such as macrophages and monocytes following administration and accumulate at active sites of inflammation (Ahrens et al. (2010) BioTechniques 50(4): 229-234). Detection of PFCs in vivo and/or ex vivo on excised samples can be performed using fluorine nuclear magnetic resonance techniques, such as ¹⁹F magnetic resonance imaging (MRI) and ¹⁹F magnetic resonance spectroscopy (MRS). Because the amount of fluorine in biological tissues is negligible, the background resonance signal is low, thus enabling highly specific detection of the accumulated PFC.

MRI is a useful modality for anatomical and functional imaging of tumors in vivo because it is not limited by field of view, tissue depth or orientation of the target organ, and provides excellent soft-tissue contrast. PFC imaging agents provide positive contrast for in vivo imaging of inflammation by MRI. One of the advantages of using perfluorocarbons as contrast agents for ¹⁹F MRI is that the fluorine atoms are detected directly. In contrast, MRI detection of other contrast agents such as superparamagnetic iron-oxide (SPIO) agents and other metal ion based contrast agents that incorporate ions, such as gadolinium and manganese, depend on the indirect effects of the isotope on surrounding water molecules, which can be affected by the large ¹H background signal. In addition, ¹⁹F MRI also is highly specific due to the naturally low amounts of fluorine in vivo. In contrast, imaging of SPIO agent is especially difficult in organs that contain high levels of iron.

Another advantage to using PFCs as imaging agents is that many PFC compounds are biologically inert. PFCs can be administered to subjects with minimal or no side effects. High doses of PFCs are generally safe in human body and pure fluorocarbon within certain molecular weight range (460-520 Da) is non-toxic, non-carcinogenic, non mutagenic and non-teratogenic and does not trigger immune responses. PFCs are widely applied in liquid ventilation, oxygen delivery and imaging due to their physiological inactivity and biocompatibility (see Marie Pierre Krafft, Advanced Drug Delivery Reviews, 47:209, 2001; Gregory et al. (2005) Current Topics in Developmental Biology 70:57). Phagocytosis of PFC compounds by inflammatory cells such as macrophages also does not negatively affect cell function. Thus, imaging by ¹⁹F MRI does not disrupt the inflammatory process, which can promote tumor therapy by the oncolytic virus.

Methods provided herein for using PFCs as MRI contrast agents involve labeling phagocytic monocyte and macrophages in vivo by active uptake of a systemically injected PFC emulsion. The methods allow tracking of the immune cell populations within the body. In the context of inflammatory processes, when large numbers of macrophages and neutrophils are recruited from the circulation and accumulate at sites of inflammation, local accumulations of the ¹⁹F signal can be observed in vivo. Such methods have been used to image, for example, in vivo inflammation after cardiac and cerebral ischemia, pulmonary inflammation in pneumonia, neuroinflammation following lysolecithin-induced nerve injury, Staphylococcus aureus infection, and inflammation following acute allograft rejection (see, e.g., Flogel et al. (2008) Circulation 118(2):140-8, Ebner et al. (2010) Circ. Cardiovasc. Imaging 3(2):202-10, Weise et al. (2011) Experimental Neurology 229:494-501, Hertlein et al. (2011) PLoS ONE 6(3):e18246, and Hitchens et al. (2011) Magnetic Resonance in Medicine 65:1145-1154). Several studies have shown that a linear relationship exists between the amount of PFCs that accumulate at the site of inflammation and the degree of inflammation in the tissue (see, e.g. Kadayakkara et al. (2012) Lab. Invest. 92(4):636-45).

Provided herein are methods of using PFC imaging agents in the context of oncolytic cancer therapy. Because the treatment of solid tumors with oncolytic viruses induces the massive recruitment of immune cells, including macrophages, to the tumor, perfluorocarbon imaging agents can be used to detect active infection of tumors by oncolytic viruses. Administration of perfluorocarbon imaging agents to tumor bearing subjects receiving oncolytic virus therapies can be used as an indirect way to detect virus delivery to a tumor and to assess the extent of viral tumor colonization. Because the effectiveness of oncolytic therapy depends on colonization of the tumor, the methods provided herein can be employed to assess the efficacy of an oncolytic virus treatment. The methods provided herein can be employed for the selection of a particular oncolytic virus for treatment. The methods provided herein also can be employed for the selection for selection of a candidate for treatment with an oncolytic virus.

As shown in the working Examples provided herein, intravenous administration of a perfluorocarbon emulsion following systemic administration of vaccinia virus resulted in the accumulation of a strong ¹⁹F signal in the tumor periphery as imaged by ¹⁹F MRI. In untreated control tumors, some accumulation of PFC also was detected, however, the spatial distribution of the PFC was diffuse throughout the tumor. ¹⁹F MRI of excised tumors confirmed the in vivo imaging results. Immunohistochemistry of the excised tumor tissue using macrophage and neutrophil-specific antibodies indicated that the ¹⁹F MRI signal pattern correlated with the histological staining pattern of the immune cells. These results demonstrated that the increase in tumor inflammation following oncolytic virus infection was detectable in vivo and ex vivo using ¹⁹F MRI of accumulated PFC.

PFC imaging agents effectively label most phagocytic immune cells including precursor cells in the bone marrow. These cells do not die due to phagocytosis of PFC and are still active. This allows for tracking of immune cell populations before and after virus administration. Accordingly, provided herein are methods for the monitoring of inflammation at the tumor prior to, during administration of, and following administration of an oncolytic virus therapy. The methods provided can be used to assess presence and extent of viral colonization as well as monitor the effects of viral colonization of the tumor over time.

Visualization of macrophage recruitment using PFC imaging agents can be employed prognostic factor for oncolytic therapy success. Immunological reaction of the host against the virus and the tumor microenvironment is an important component of the anti-tumor response in the patient. Local recruitment of macrophages is an early step of the host response to oncolytic virus therapy and can be an indicator of whether a particular tumor can be colonized by the virus. Thus, the methods provided herein can be used to determine whether the tumor will respond to an oncolytic viral therapy.

The methods provided herein include systemic (e.g. intravenous) administration of PFC imaging agents to a tumor-bearing subject and detection of the PFC. Detection can be performed in vivo using ¹⁹F magnetic resonance imaging (MRI) to detect the accumulation of the PFC at the tumor. ¹⁹F MRI or ¹⁹F MRS also can be performed on extracted tumor tissue (e.g. a biopsy) to detect the PFC.

Exemplary oncolytic viruses for use in the methods include diagnostic and therapeutic viruses. Exemplary oncolytic viruses are provided herein and include, but are not limited to, poxvirus, adenovirus, reovirus, herpes virus, adeno-associated virus, lentivirus, retrovirus, rhabdovirus, papillomavirus, vesicular stomatitis virus, measles virus, Newcastle disease virus, picornavirus, sindbis virus, papillomavirus, parvovirus, coxsackievirus, influenza virus, mumps virus, poliovirus and semliki forest virus. Exemplary poxviruses include, but are not limited to, viruses selected from among vaccinia virus, orthopoxvirus, parapoxvirus, avipoxvirus, capripoxvirus, leporipoxvirus, suipoxvirus, molluscipoxvirus, yatapoxvirus, entomopoxvirus A, entomopoxvirus B and entomopoxvirus C. Exemplary vaccinia viruses include Lister strain of vaccinia viruses, including, but not limited to, LIVP strains of vaccinia viruses.

### C. METHODS FOR IMAGING TUMOR INFLAMMATION INDUCED BY ONCOLYTIC VIRUSES

Provided herein are methods of detecting and monitoring infection of a tumor by an oncolytic virus. The methods provided are employ the ability of virus infection and replication to induce the recruitment of inflammatory cells, such as macrophages, to the tumor. The induction of tumor inflammation can be detected in vivo in a subject or ex vivo in a tumor sample from a subject using any agent that detects macrophage particularly imaging or other detectable agents. Exemplary of such agents are perfluorocarbon (PFC) imaging agents. Perfluorocarbon (PFC) imaging agents that are taken up by the inflammatory cells and detectable via nuclear magnetic resonance methods and/or other imaging methods as described herein. The methods can be employed to assess the presence of or level of tumor inflammation prior to, during, or following administration of an oncolytic virus therapy.

Also provided are methods for diagnosing, detecting or imaging tumors by administering oncolytic viruses, such as vaccinia viruses, where the viruses do not encode a heterologous detectable reporter protein or a protein that induces a detectable signal. Thus, the viruses can be unmodified viruses, or viruses that encode therapeutic proteins, but they do not need to encode a protein for detection. Administration of the virus leads to an inflammatory response, and the influx of macrophage. Any agent for detection of macrophage, such as a perfluorocarbon imaging agents, will detect the tumors by virtue of the influx of macrophage resulting from accumulation of the viruses. Thus, provided are indirect methods for detection of oncolytic viruses. Detection of oncolytic viruses permits detection, particularly imaging, of tumors, monitoring of therapy, assessment of colonization of tumors by oncolytic viruses and, thus, assessment of the potential effectiveness of an oncolytic virus for therapy for a particular tumor or subject. An advantage is that the viruses do not need to be adapted for detection.

1. Exemplary Methods for Imaging Tumor Inflammation

In exemplary methods, a PFC imaging agent is administered to a tumor-bearing subject and the accumulation of the PFC in the tumor is detected or imaged, thereby detecting presence of tumor inflammation and or the level of tumor inflammation. The PFC imaging agent can be administered prior to, at the same time as, or following the administration of an oncolytic virus.

In some examples, an oncolytic virus is first administered to a tumor-bearing subject and, at a predetermined time thereafter, a PFC imaging agent is administered to the subject. Following administration of the PFC imaging agent, the accumulation of the PFC in the tumor is detected or imaged.

In some examples, a PFC imaging agent is first administered to a tumor-bearing subject and, at a predetermined time thereafter, an oncolytic virus is administered to the subject. Following administration of the oncolytic virus, the accumulation of the PFC in the tumor is detected or imaged.

In some examples, a PFC imaging agent is administered at the same time as an oncolytic virus. Following administration of the oncolytic virus with the PFC imaging agent, the accumulation of the PFC in the tumor is detected or imaged. In such examples, the oncolytic virus and the PFC imaging agent can be administered as a single composition or as two separate compositions.

In some examples, detection of the PFC signal at the tumor periphery indicates that the oncolytic virus has infected the tumor. In some examples, detection of the PFC signal as a diffuse signal throughout the tumor indicated that the oncolytic virus has not infected the tumor.

In some examples, the induction of tumor inflammation by an oncolytic virus is determined by comparing the level of tumor inflammation prior to administration of the virus to the level of tumor inflammation following administration of the oncolytic virus. An increase in the amount of tumor inflammation, as evidenced by an increase in the accumulation of PFC at the tumor, indicates that the oncolytic virus has infected the tumor. In such examples, a PFC imaging agent is administered to a tumor-bearing subject at a predetermined time prior to administration of the oncolytic virus and the accumulation of the PFC in the tumor is detected or imaged. The oncolytic virus is then administered to the subject, and the accumulation of the PFC in the tumor is detected or imaged. An increase in the accumulation of the PFC at the tumor indicates that the level of tumor inflammation has increased and that the oncolytic virus has infected the tumor.

In some examples, the level of tumor inflammation following administration of an oncolytic viruses is monitored over time. For example, accumulation of the PFC at the tumor can be detected or imaged at multiple time points following administration of the oncolytic virus, including, for example, at 2, 3, 4, 5, 6, 7, 8, 9, 10, 15, 20 or more time points following administration of the oncolytic virus.

In some examples, the induction of tumor inflammation by an oncolytic virus is determined by comparing the level of tumor inflammation following administration of the oncolytic virus to the level of tumor inflammation in a control. Exemplary controls include, but are not limited to a control tumor sample or in vivo or in vitro data from a subject who has been administered a PFC imaging agent but not the oncolytic virus therapy or can be a control tumor sample or in vivo or in vitro data from the test subject prior to receiving the oncolytic virus. In some examples, the control is a positive control sample a tumor sample or in vivo or in vitro data from a subject who has been administered a PFC imaging agent and an oncolytic virus therapy.

The oncolytic viruses used in the methods provided can be administered to the subject by any suitable method for administering a diagnostic or therapeutic oncolytic virus. Administration of oncolytic viruses to a subject, including a human subject or non-human mammalian subject, is well-known in the art. The oncolytic virus can be administered by any suitable route. For example, the oncolytic viruses can be administered to the subject systemically or locally to the tumor. Exemplary routes of administration include, but are not limited to intravenous, intraarterial, intratumoral, endoscopic, intralesional, intramuscular, intradermal, intraperitoneal, intravesicular, intraarticular, intrapleural, percutaneous, subcutaneous, oral, parenteral, intranasal, intratracheal, inhalation, intracranial, intraprostatic, intravitreal, topical, ocular, vaginal, or rectal routes of administration. In particular examples, the oncolytic viruses are administered intraperitoneally or intravenously.

The dosage regimen can be any of a variety of methods and amounts, and can be determined by one skilled in the art according to known clinical factors. As is known in the medical arts, dosages for any one subject can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art. In the present methods, appropriate minimum dosage levels of viruses can be levels sufficient for the virus to survive, grow and replicate in a tumor or metastasis. Exemplary minimum levels for administering a virus to a 65 kg human can include at least or about 1×10² plaque forming units (PFU), at least or about 1×10³ plaque forming units (PFU), at least or about 1×10⁴ plaque forming units (PFU), at least or about 1×10⁵ plaque forming units (PFU), at least about 5×10⁵ PFU, at least about 1×10⁶ PFU, at least about 5×10⁶ PFU, at least about 1×10⁷ PFU, at least about 1×10⁸ PFU, at least about 1×10⁹ PFU, or at least about 1×10¹⁰ PFU. In the present methods, appropriate maximum dosage levels of viruses can be levels that are not toxic to the host, levels that do not cause splenomegaly of 3 times or more, levels that do not result in colonies or plaques in normal tissues or organs after about 1 day or after about 3 days or after about 7 days. Exemplary maximum levels for administering a virus to a 65 kg human can include no more than about 1×10¹⁴ PFU, no more than about 1×10¹³ PFU, no more than about 1×10¹² PFU, no more than about 1×10¹¹ PFU, no more than about 5×10¹⁰ PFU, no more than about 1×10¹⁰ PFU, no more than about 5×10⁹ PFU, no more than about 1×10⁹ PFU, or no more than about 1×10⁸ PFU.

In some examples, the oncolytic virus is administered in an amount sufficient to induce accumulation of the perfluorocarbon at the tumor where such dosage that is lower than a treatment dosage of the virus. For example, exemplary dosages include, but are not limited to, a dosage at or about 1×10² pfu to at or about 1×10⁸ pfu, such as, for example, at or about 1×10² pfu, 1×10³ pfu, 1×10⁴ pfu, 1×10⁵ pfu, 1×10⁶ pfu, 1×10⁷ pfu or 1×10⁸ pfu. In some examples, the oncolytic virus is administered at a dosage for treatment of a tumor or cancer. For example, exemplary dosages include, but are not limited to, a dosage at or about 1×10⁶ pfu to at or about 1×10¹⁴ pfu, such as, for example, at or about 1×10⁶ pfu, 1×10⁷ pfu or 1×10⁸ pfu, 1×10⁹ pfu, 1×10¹⁰ pfu, 1×10¹¹ pfu, 1×10¹² pfu, 1×10¹³ pfu, or 1×10¹⁴ pfu.

Generally, the PFC imaging agent is administered in a manner that is suitable for uptake by phagocytic cells of the immune system. Typically, the PFC imaging agent for use in the methods provided is in the form of an emulsion and is administered systemically, such as for example, intravenously. The PFC imaging agent can be administered prior to, at the same time as, or following administration of an oncolytic virus. In some examples, the PFC imaging agent is administered at a time following administration of an oncolytic virus sufficient for the virus to have accumulated in the tumor and/or induce inflammation at the tumor. In some examples, the PFC imaging agent is administered at a time following systemic administration of an oncolytic virus sufficient for the virus to be cleared from the bloodstream. In some examples, the PFC imaging agent is administered 1 hour, 2 hours, 3 hours, 4 hours, 6 hours, 12 hours, 18 hours, 24 hours, 2 days, 3 days, 4 days, 5 days, 6 days, 7 days, 8 days, 9 days, 10 days, 11 days, 12 days, 13 days, 14 days, 3 weeks 4 weeks, 1 month or longer following administration of the oncolytic virus. In some examples, the PFC imaging agent is administered at the same time as the oncolytic virus. In some examples, the PFC imaging agent is administered 1 hour, 2 hours, 3 hours, 4 hours, 6 hours, 12 hours, 18 hours, 24 hours, 48 hours, or longer prior to the administration of the oncolytic virus.

Generally, the amount of a PFC agent administered is sufficient for detection in vivo by ¹⁹F MRI at a tumor. High doses of PFCs are well-tolerated in humans and generally do not cause side effects. For use as an oxygen carrier, doses of PFC at 500 g were well-tolerated in human patients. Typically, side effects resulting from administration of PFC emulsions are due to the emulsification agent and can be controlled by selection of non-toxic or low toxicity emulsification agent for the production of the emulsion. Exemplary dosages of PFC imaging agent for administration include, but are not limited to, 0.1 g, 1 g, 5 g, 10 g, 20, 30 g, 40 g, 50 g, 100 g, 150 g, 200 g, 250 g, 300 g, 350 g, 400 g, 450 g, 500 g, 550 g, 600 g, 650 g, 700 g, 800 g, 850 g, 900 g, 950 g, 1000 g or more of the perfluorocarbon in an emulsion per average 65 kg human. Exemplary emulsion volumes for administration include, but are not, limited to, 0.05 ml/kg, 1 ml/kg, 2 ml/kg, 3 ml/kg, 4 ml/kg, 5 ml/kg, 6 ml/kg, 7 ml/kg, 8 ml/kg, 9 ml/kg, 10 ml/kg, 15 ml/kg, 20 ml/kg, 25 ml/kg, 30 ml/kg, 35 ml/kg, 40 ml/kg, 45 ml/kg, 50 ml/kg of the emulsion.

Typically, the tumor is detected or imaged at a time following systemic (e.g. intravenous) administration of the PFC imaging agent sufficient to clear the PFC imaging agent from the blood stream and accumulate at the site of tumor inflammation. In some examples, the tumor is detected or imaged 1 hour, 2 hours, 4 hours, 6 hours, 12 hours, 18 hours, 24 hours, 30 hours, 36 hours, 42 hours, 48 hours or later following administration of the PFC imaging agent. In particular examples, the tumor is imaged at about 24 to at about 48 hours following administration of the PFC imaging agent. In some examples, the tumor is detected or imaged at two or more time points following administration of the PFC imaging agent, including, for example, at 2, 3, 4, 5, 6, 7, 8, 9, 10, 15, 20 or more time points. Such time points can be prior to, at the same time as, or following administration of an oncolytic virus.

In some examples, the oncolytic virus is administered according to a dosage regimen and the induction of tumor inflammation is assessed following each successive cycle or multiple cycles of administration or during the cycle of administration at one or more predetermined time points following administration of the oncolytic virus. Exemplary lengths of time between successive cycles of administration of the virus include, but are not limited to, at least or about two days, three days, four days, five days, six days, seven days, 14 days, 21 days or 28 days. In some examples, the oncolytic virus is administered in an amount that is at least 1×10⁹ pfu at least one time over a cycle of administration. In some examples, the oncolytic virus is administered one time during a cycle of administration. In some examples, the oncolytic virus is administered a plurality of times during a cycle of administration, such as, for example, two times, three times, four times, five times, six times or seven times over the cycle of administration. In some examples, the virus is administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle.

In some examples, the methods of detecting or imaging tumor inflammation can be used in combination with a method to detect or image expression of a reporter gene encoded by the oncolytic virus. Exemplary methods of detecting expressed reporter proteins are provided elsewhere herein and include, but are not limited to fluorescent, luminescent, spectrophotometric, chromogenic assays, acoustic/ultrasonic detection, or radioactive detection methods.

a. Exemplary Subjects

Generally, the methods provided herein can be performed on any subject that has a tumor. Typically, the tumor is a solid tumor. In some examples, the tumor is a metastatic tumor. In some examples, the subject has a pre-cancerous lesion (dysplasia), carcinoma, adenocarcinoma, or a sarcoma. In some examples, the subject has a tumor and is at risk of metastasis of the tumor. In some examples, the subject has an advanced stage cancer.

In some examples, the subject has a cancer that is adenocarcinoma, adenoma, adrenal cancer, adrenocortical carcinoma, AIDS-related cancer, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, carcinoid tumor, carcinoma, cervical cancer, colon cancer, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenström's macroglobulinemia or Wilm's tumor. In particular examples, the cancer is a cancer of the bladder, brain, breast, bone marrow, cervix, colon/rectum, kidney, liver, lung/bronchus, ovary, pancreas, prostate, skin, stomach, thyroid, or uterus. The methods provided herein can be used to image numerous tumors including, but not limited to, any of the tumors described herein.

In some examples, the subject is mammal. Exemplary mammalian subjects include, but are not limited to primates, such as humans, apes and monkeys; rodents, such as mice, rats, rabbits, and ferrets; ruminants, such as goats, cows, deer, and sheep; horses, pigs, dogs, cats, and other animals. In some examples, the subject is patient. In some examples, the patient is a human patient.

2. Perfluorocarbon Imaging Agents for Use in the Methods

a. Perfluorocarbon Emulsions

Typically, the perfluorocarbon (PFC) imaging agents for use in the methods provided herein are in the form of an emulsion containing a PFC and one or more emulsification and/or emulsion stabilization agents. The compositions also can contain additional components such as additional surfactants, including additional emulsifiers or emulsion stabilization agents, lipids, additives, therapeutic agents or diagnostic agents. Exemplary PFCs for use in a PFC emulsion are provided herein. In some examples, the PFC emulsion employed in the methods provided is a commercially available PFC emulsion, such as, for example, V-Sense 580H (30% v/v perfluoro-15-crown-5-ether), V-Sense 1000H (20% v/v perfluoro-15-crown-5-ether) or V-Sense DM Green (CelSense, Pittsburgh, Pa.) or Fluorovist® (HemaGen/PFC, St. Louis, Mo.). Exemplary PFC emulsions that can be used in the methods provided also are described in, for example, U.S. Patent App. Pub. Nos. US2009/0280055, US2007/0253910, now U.S. Pat. No. 8,263,043, US2008/0292554, now U.S. Pat. No. 8,147,806, US2011/0110863, US 2009/0074673, now U.S. Pat. No. 8,227,610, US 2009/0263329.

i. Exemplary Perfluorocarbons (PFCs)

The perfluorocarbon (PFC) imaging agents for use in the methods provided herein contain one or more perfluorocarbons (PFCs). Generally, the perfluorocarbon contains a plurality of fluorine atoms bound to carbon, such as for example, 5, 10, 15, 20 fluorine atoms in the molecule bound to carbon. In some examples, the PFC imaging agent contains a perfluorocarbon that is a linear or branched chain perfluorocarbon. In some examples, the PFC imaging agent contains a perfluorocarbon that is a perfluorocarbon is a cyclic perfluorocarbon. Exemplary perfluorocarbons include, but are not limited to, perfluoroethers, such as perfluoropolyethers (PFPEs) and perfluoro-crown ethers (PFCEs), including but not limited to, perfluoro-15-crown-5-ether, perfluoro-18-crown-6-ether and perfluoro-12-crown-4-ether, perfluoroalkylethers, perfluoroalkanes such as, but not limited to, perfluoropentane (PFP), perfluorohexane (PFH), perfluorooctane, perfluorononane, perfluorohexyl bromide, perfluorooctyl bromide (PFOB), and perfluorodecyl bromide; perfluoroalkenes such as, but not limited to, bisperfluorobutylethylene; perfluorocycloalkanes such as perfluorodecalin, perfluorocyclohexanes, perfluoroadamantane, perfluorobicyclodecane, and perfluoromethyl decahydroquinoline; perfluoro amines such as perfluoroalkyl amines; and C1-C8 substituted compounds thereof, isomers thereof, and combinations thereof. Additional exemplary PFCs for use in a perfluorocarbon (PFC) imaging agent for use in the methods provided herein include, but are not limited to, PFCs described in, U.S. Patent App. Pub. Nos. US 2009/0074673, now U.S. Pat. No. 8,227,610, and US 2010/0233094. In particular examples, the perfluorocarbon is a perfluoro-crown ether, such as, for example, perfluoro-15-crown-5-ether.

(1) Modified Perfluorocarbons

In some examples, the perfluorocarbon imaging agent for use in the methods provided herein contains a perfluorocarbon that is a modified perfluorocarbon. In some examples, the modified perfluorocarbon contains an additional moiety. In some examples, the additional moiety is a functional moiety. In some examples, the functional moiety is a detectable moiety and/or a therapeutic moiety. In some examples, the detectable moiety is detectable by a method other than a nuclear magnetic resonance technique. In some examples, the detectable moiety is a fluorescent moiety or a PET moiety. In some examples, the functional moiety is therapeutic agent such as a radionuclide, chemotherapeutic agent or toxin useful for the treatment or a tumor.

In some examples, the perfluorocarbon imaging agents used in the methods are detectable by more than one imaging method. For example, the perfluorocarbon imaging agent can be detectable by a nuclear magnetic resonance methods and another method, such as, but not limited, to fluorescent imaging, positron emission tomography (PET), or ultrasonography. In some examples, the perfluorocarbon is a modified perfluorocarbon that contains one or more ¹⁸F isotopes for detection of the fluorocarbon by positron emission spectroscopy. Exemplary modified perfluorocarbon contain a mixture of ¹⁸F and ¹⁹F isotopes, thus providing a dual mode label that is suitable for MRI/MRS and PET. ¹⁸F and ¹⁹F may also be added in separate monomers to form a mixed copolymer, or ¹⁸F portions may be located at either end of a linear polyether, at the position where most other functional moieties would be added. ¹⁸F has no NMR signal and so may be added at positions that would, for example, tend to decrease NMR linewidth, simplify the NMR spectrum, or alleviate chemical shifts from resonances that adversely affect the read-out obtained by a nuclear magnetic resonance technique. In addition, perfluorocarbons can incorporate other radioisotopes that are effective PET probes, such as ¹¹C, ¹⁵O, and ¹³N. Those skilled in the art can, in view of this specification, devise many other PET-detectable moieties that can be incorporated into or, for example, attached to an endgroup(s), of the PFC imaging reagents. In some examples, the perfluorocarbon is a modified perfluorocarbon that contains a quantum dot nanoparticle attached to a perfluorocarbon (see, e.g. U.S. Patent App. Pub. No. 2010/0233094).

In some examples, the perfluorocarbon is a modified perfluorocarbon that is conjugated to a fluorophore. Many suitable fluorophores are known in the art, including fluorescein and its derivatives (e.g., Oregon Green 488 and 514, Dichlorofluorescein, Carboxyfluorescein, etc., where all are available from Molecular Probes), lissamine, phycoerythrin, rhodamine (Perkin Elmer Cetus), Cy2, Cy3, Cy3.5, Cy5, Cy5.5, Cy7, Alexa dyes (Molecular Probes), BODIPy dyes (Molecular Probes) and Fluor X (Amersham). Fluorescent moieties include derivatives of fluorescein, benzoxadioazole, coumarin, eosin, Lucifer Yellow, pyridyloxazole and rhodamine. These and many other exemplary fluorescent moieties are described in the Handbook of Fluorescent Probes and Research Chemicals (2000, Molecular Probes, Inc.).

In some examples, the perfluorocarbon is a modified perfluorocarbon that is conjugated to a targeting agent, such as a ligand or a receptor. In some examples, the perfluorocarbon is a modified perfluorocarbon that is conjugated to an internalization peptide, such as antepennepedia protein, HIV transactivating peptide (TAT), mastoparan, melittin, bombolitten, delta hemolysin, paradaxin, Pseudomonas exotoxin A, clathrin, Diptheria toxin, C9 complement protein or a fragment thereof.

ii. Selection of Perfluorocarbons

Perfluorocarbons for use in MRI imaging generally are selected based on the magnetic properties of the fluorine atoms of the compound. PFCs containing magnetically equivalent fluorine atoms are advantageous because the atoms resonate at the same frequency. Typically, a perfluorocarbon with a narrow bandwidth resonance frequency that is distinct. In some examples, the PFC possesses a single narrow bandwidth resonance frequency. For example, the PFC perfluoro-15-crown-5-ether possesses a single narrow resonance frequency.

Generally, the PFC selected is non-toxic (i.e. the perfluorocarbon can be administered to a subject and cause minimal or no side effects). Generally, the PFC imaging agent must be able to be phagocytosed by a monocyte or a macrophage. The selected PFC generally does not affect the biological properties of the macrophage following phagocytosis of the compound. In some examples, the PFC imaging agent does not incorporate into cell membranes.

In particular examples, the PFC imaging agent contains two or more PFCs and can be detected at distinct resonance frequencies. In such examples, the PFCs are typically selected such that ¹⁹F MRI signals derived from each PFC do not interfere with one another. In particular examples, where the PFC imaging agent contains two or more PFCs, the PFCs are selected such that ¹⁹F MRI signals derived from each perfluorocarbon are distinct (i.e. the signal bandwidth resonance frequency of one PFC does not overlap with any resonance frequency of another PFC). In some examples, each PFC can be detected independently based on distinct resonance.

b. Emulsification and Emulsion Stabilization Agents

Emulsification agents aid in the formation of PFC nanoparticles of the emulsion and stabilize the emulsion. Exemplary emulsification agents, include, but are not limited to, nonionic surfactants, cationic surfactants, anionic surfactants, and amphoteric surfactants.

In particular examples, the emulsification agent or emulsion stabilization agent is a block copolymer. Non-limiting examples of suitable poly(ethylene oxide)-polyester block copolymers include poly(ethylene oxide) block copolymers with ε-caprolactone, (L or D,L) lactide, D,L-lactic acid, D-lactic acid, L-lactic acid, glycolide, glycolic acid, lactic and glycolic acid (PLGA), hydroxy hexanoic acid, γ-butyrolactone, γ-hydroxy butyric acid, δ-valerolactone, hydroxy valeric acid, hydroxybutyric acids, malic acid, copolymers thereof, and combinations thereof.

In particular examples, the emulsification agent or emulsion stabilization agent is a nonionic triblock copolymer. In particular examples, the nonionic triblock copolymer is a poloxamer (i.e. a Pluronic™) that contains a central hydrophobic chain of poly(propylene oxide) (PPO, polyoxypropylene) flanked by two hydrophilic chains of poly(ethylene oxide) (PEO, polyoxyethylene) (i.e. PEO-PPO-PEO). In particular examples, the PEO-PPO-PEO triblock copolymer has an average molecular weight of 1900, 2900, 6500, 8400. In a particular example, the PEO-PPO-PEO triblock copolymer has an average molecular weight of 1900 and a hydrophilic-lipophilic balance (HLB) value of about 19. In a particular example, the PEO-PPO-PEO triblock copolymer has an average molecular weight of 2900 and a hydrophilic-lipophilic balance (HLB) value of about 15. In a particular example, the PEO-PPO-PEO triblock copolymer has an average molecular weight of 6500 and a hydrophilic-lipophilic balance (HLB) value of about 15. In a particular example, the PEO-PPO-PEO triblock copolymer has an average molecular weight of 8400 and a hydrophilic-lipophilic balance (HLB) value of about 29. Exemplary poloxamers that can be used to generate a PFC emulsion include, but are not limited to, Pluronic™ L-35, Pluronic™ L64, Pluronic™ P105, Pluronic™ F-68, and Pluronic™ F-127 (BASF Corporation).

Additional exemplary emulsification agents that can be used to generate PFC emulsions, include, but are not limited to, polyvinyl alcohol (PVA), Hamposyl™ L30, sodium dodecyl sulfate, Aerosol 413, Aerosol 200, Lipoproteol™ LCO, Standapol™ LCO, Standapol™ SH 135, Fizul™ 10-127, Cyclopol™ SBFA 30, Deriphat™ 170, Lonzaine™ JS, Niranol™ C2N-SF, Amphoterge™ W2, Amphoterge™ 2WAS, Brij™ 35, Triton™ X-100, Brij™ 52, Span™ 20, Generol™ 122 ES, Triton™ N-42, Triton™ N-101, Triton™ X-405, Tween™ 80, Tween™ 85, and Brij™ 56.

In some examples, the emulsion contain an emulsion stabilization agent. In some examples, the emulsion stabilization agent also is emulsification agent.

In some examples, the PFC emulsion contains one or more lipid for stabilization of the emulsion. In particular examples, the lipid is mixed with the emulsification agent to enhance stabilization of the emulsion. In some examples, the lipid can be coupled to a targeting agent. Exemplary lipids include, but are not limited to, natural or synthetic phospholipids, fatty acids, cholesterols, lysolipids, sphingomyelins, tocopherols, glucolipids, stearylarginines, cardiolipins, plasmalogens, a lipid with ether or ester linked fatty acids, polymerized lipids, and a mixture thereof. In a particular example, the lipid in the PFC emulsion is dimyristoylphosphatidylcholine (DMPC).

c. Characteristics of Perfluorocarbon Imaging Agent Emulsions

Generally, the mean particle size of the nanoparticles of the emulsion is less than or about 800 nm, such as, for example, less than or about 750 nm, less than or about 700 nm, less than or about 650 nm, less than or about 600 nm, less than or about 550 nm, less than or about 500 nm, less than or about 450 nm, than or about 400 nm, than or about 350 nm, than or about 300 nm, than or about 250 nm, than or about 200 nm, than or about 150 nm, than or about 100 nm, than or about 50 nm, or than or about 25 nm. Typically, the mean particle size of the nanoparticles of the emulsion is less than or about 500 nm. In particular examples, the PFC droplet size in the emulsion is about 145 nm. Typically, the polydispersity index of the emulsion ranges from about 0.1 to about 0.2.

The amount of the PFC in the emulsion relative other components of the emulsion can be varied. Exemplary percentages of PFC in an emulsion by volume include, but are not limited to, at or about 5% v/v, at or about 10% v/v, 15% v/v, 20% v/v, 25% v/v, 30% v/v, 35% v/v, 40% v/v, 45% v/v, 50% v/v, 55% v/v, 60% v/v, 65% v/v, 70% v/v, 75% v/v, 80% v/v, 85% v/v, 90% v/v, and 95% v/v. In some examples the perfluorocarbon is present in the emulsion from about 5% v/v to about 60% v/v of the emulsion, such as, for example, 10% v/v to about 40% v/v. In particular examples, the PFC is 20% v/v of the emulsion. In particular examples, the PFC is perfluoro-15-crown-5-ether and the PFC is 20% v/v of the emulsion. In particular examples, the PFC is 30% v/v of the emulsion. In a particular example, the PFC is perfluoro-15-crown-5-ether and the PFC is 30% v/v of the emulsion. In a particular example, the emulsion is V-Sense 1000H (CelSense Inc., Pittsburgh, Pa.). In particular examples, the PFC is 40% v/v of the emulsion. In particular examples, the PFC is perfluorooctylbromide and the PFC is 40% v/v of the emulsion.

Exemplary percentages of PFC in an emulsion by weight include, but are not limited to, at or about 5% wt/wt, at or about 10% wt/wt, 15% wt/wt, 20% wt/wt, 25% wt/wt, 30% wt/wt, 35% wt/wt, 40% wt/wt, 45% wt/wt, 50% wt/wt, 55% wt/wt, 60% wt/wt, 65% wt/wt, 70% wt/wt, 75% wt/wt, 80% wt/wt, 85% wt/wt, 90% wt/wt, and 95% wt/wt. In some examples the perfluorocarbon is present in the emulsion from about 5% v/v to about 60% v/v of the emulsion, such as, for example, 10% wt/wt to about 40% wt/wt.

d. Addition of Therapeutic and Diagnostic Agents

The PFC imaging agents for use in the methods provided herein can additionally contain one or more therapeutic agents and/or one or more additional diagnostic agents for delivery to a tumor. The therapeutic and/or diagnostic agents can be used for therapy and or detection or monitoring of a tumor. Therapeutic agents that can be included in a PFC emulsion include, but are not limited to chemotherapeutic agents, hormones, or any other biologically or chemically active agents, including nucleic acids, peptides, proteins, antibodies, and/or radionuclides that can be used to treat a condition such as various tumors and cancers. In some aspects, the therapeutic agent can include, but is not limited to, paclitaxel, doxorubicin, gemcitabine, adriamycin, cisplatin, taxol, methotrexate, 5-fluorouracil, betulinic acid, amphotericin B, diazepam, nystatin, propofol, testosterone, estrogen, prednisolone, prednisone, 2,3 mercaptopropanol, progesterone, multi-drug resistant (MDR) suppressing agents, or any combination thereof. In a non-limiting example, the PFC emulsion contains paclitaxel (PTX). A non-limiting example, the PFC emulsion is a perfluoro-15-crown-5-ether (PFCE) emulsion containing paclitaxel (PTX) (see Rapoport et al. (2011) J Control Release 153(1):4-15).

PFC emulsions carrying therapeutic and diagnostic agents can be delivered to the tumor and released by ultrasound induced (e.g. sonication with 1-MHz therapeutic ultrasound) acoustic vaporization which induces cavitation of the nanoparticles of the emulsion (see, e.g. U.S. Patent App. Pub. No. 2010/0178305).

e. Preparation of Perfluorocarbon Emulsions

The perfluorocarbon emulsions can be produced by any variety of standard methods known in the art for the generation of nanoemulsions. Exemplary methods for the generation of perfluorocarbon emulsions are described in, for example, U.S. Pat. Nos. 4,990,283, 5,330,681, 5,690,907; 5,780,010; 5,989,520; 5,958,371; U.S. Patent Pub. Nos. US 2010/0233094, US 2009/0280055, US 2007/0253910, US2008/0292554, now U.S. Pat. No. 8,147,806, and US 2011/0110863, US 2009/0074673, US2009/0263329; and PCT publication WO 02/060524

Exemplary methods for generation of a PFC emulsion include high energy and low energy methods. In some examples, the nanoemulsion is a produced by a high energy method. Exemplary high energy methods include, but are not limited to, sonication, high pressure homogenization, high shear agitation and microfluidization. In some examples, the nanoemulsion is a produced by a low energy method. An exemplary low energy method includes, but is not limited to, such as such as vortexing or a thin film method (see, e.g., U.S. Patent Pub. No. US 2010/0233094).

Equipment, such as homogenizer and emulsifiers for the production of emulsions are readily available in the art. Exemplary equipment for the production of emulsions includes, but is not limited to, Sonifier Cell disruptor, Emulsiflex C5 (Avestin), PowerGen 1000 homogenizer (Fisher Scientific), and Model M-1105 emulsifier/Microfluidizer (Microfluidics Co.). The resulting emulsion can further be sterilized, for example, by autoclaving or passing through a suitable filter.

The properties of prepared emulsions can be determined. For example, particle size, stability and purity of the emulsions can be measured. Particle size can be measured, for example by dynamic light scattering (e.g. Malvern Zetasizer Nano, Malvern, Worcestershire, UK or Zetatrac™ Particle Metrix, Meerbusch, Germany). Additionally, dual imaging PFC agents that contain dyes or fluorescent moieties can be analyzed by standard methods including, but not limited to, chromogenic assays, spectroscopy and fluorescence microscopy.

3. Detection and Imaging Methods

a. In Vivo Detection and Imaging

Detection and imaging of PFC accumulation generally is performed by MRI. MRI examination may be conducted according to any suitable methodology known in the art. Many different types of MRI pulse sequences, or the set of instructions used by the MRI apparatus to orchestrate data collection, and signal processing techniques (e.g. Fourier transform and projection reconstruction) have been developed over the years for collecting and processing image data (for example, see Magnetic Resonance Imaging, Third Edition, editors D. D. Stark and W. G. Bradley, Mosby, Inc., St. Louis Mo. 1999). The reagents and methods provide are not tied to any particular imaging pulse sequence or processing method of the raw NMR signals. For example, MRI methods that can be applied in the methods provided broadly encompasses spin-echo, stimulated-echo. gradient-echo, free-induction decay based imaging, and any combination thereof. Fast imaging techniques, where more than one line in k-space or large segments of k-space are acquired from each excited signal, are also highly suitable to acquire the ¹⁹F (or ¹H) data. Examples of fast imaging techniques include fast spin-echo approaches (e.g. FSE, turbo SE, TSE, RARE, or HASTE), echo-planar imaging (EPI), combined gradient-echo and spin-echo techniques (e.g. GRASE), spiral imaging, and burst imaging. The development of new and improved pulse sequence and signal processing methods is a continuously evolving field, and persons skilled in the art can devise multiple ways to image the ¹⁹F labeled cells in their anatomical context. Exemplary methods for imaging perfluorocarbons in vivo by MRI are described in, for example, U.S. Patent App. Pub. Nos. US 2009/0280055, US 2007/0253910, US 2008/0292554, US 2011/0110863, US 2009/0074673, and US 2009/0263329.

For MRI, the tumor can be imaged in vivo using a magnetic resonance imaging system with surface coils adjustable to ¹⁹F and ¹H resonance frequencies. ¹H MRI provides an anatomical image of tumor and surrounding tissues that can be compared to or overlayed with the ¹⁹F signal image. Acquisition of ¹H anatomical images allows precise determination of the anatomical location of ¹⁹F signal within subject, including the precise location within the tumor. Suitable equipment for detection of ¹⁹F and ¹H is available in the art. In some examples, the tumor is imaged using a 7T Bruker Biospec System (Bruker BioSpin GmbH, Reinstetten, Germany). Typically, the MRI methods is performed at room temperature. Exemplary parameters for ¹⁹F and ¹H MRI can be found in the examples and in the art as described in U.S. Patent App. Pub. Nos. US 2009/0280055, US 2007/0253910, US 2008/0292554, US 2011/0110863, US 2009/0074673, and US 2009/0263329 and patents based thereon. In some examples, quantification of the signal is performed using a computer and imaging software.

b. Ex Vivo Detection and Imaging

Tumor inflammation also can be determined ex vivo in a sample obtained from the subject. In some examples, a tumor sample (i.e. biopsy) is obtained from a subject at a predetermined time following administration of an oncolytic virus. Detection of PFC accumulation in the sample can be determined by a nuclear resonance methods such as, for example, ¹⁹F MRI or ¹⁹F MRS. Exemplary methods for extraction and imaging of tumor tissue by ¹⁹F MRI and ¹⁹F MRS are described in the Examples provided and in Ahrens et al. (2005) Nat Biotechnol 23(8):983-987.

In some examples, the sample is a tissue biopsy and is obtained, for example, by needle biopsy, CT-guided needle biopsy, aspiration biopsy, endoscopic biopsy, bronchoscopic biopsy, bronchial lavage, incisional biopsy, excisional biopsy, punch biopsy, shave biopsy, skin biopsy, bone marrow biopsy, and the Loop Electrosurgical Excision Procedure (LEEP). Typically, a non-necrotic, sterile biopsy or specimen is obtained that is greater than 100 mg, but which can be smaller, such as less than 100 mg, 50 mg or less, 10 mg or less or 5 mg or less; or larger, such as more than 100 mg, 200 mg or more, or 500 mg or more, 1 gm or more, 2 gm or more, 3 gm or more, 4 gm or more or 5 gm or more. The sample size to be extracted for the assay can depend on a number of factors including, but not limited to, the number of assays to be performed, the health of the tissue sample, the type of cancer, and the condition of the patient. The tissue is typically placed in a sterile vessel, such as a sterile tube or culture plate, and can be optionally immersed in an appropriate media.

Additional analysis of the ex vivo sample can be performed, such as, for example, immunohistochemistry or PCR analysis of gene expression for detection of particular cell types in the sample. In some examples, immunohistochemical analysis of macrophage specific markers, such as CD68, or other immune cell specific markers can be examined. In some examples, the sample can be analyzed for the expression of a detectable gene encoded by the virus, such as a fluorescent or luminescent protein or other detectable gene product. In some examples, the sample can be analyzed for the detectable moieties conjugated to a modified perfluorocarbon such as for example, fluorescent moieties, PET imaging moieties (e.g. ¹⁸F) or other imaging moieties as described herein.

### D. VIRUSES FOR USE IN THE METHODS

1. Exemplary Oncolytic Viruses

The viruses for use in the methods provided herein typically are replication competent viruses that can selectively infect neoplastic cells (i.e. oncolytic viruses). Numerous oncolytic viruses have been identified or developed. These include vaccinia viruses, vesicular stomatitis viruses, herpes viruses, measles viruses and adenoviruses. Such viruses have been employed for the detection, imaging and therapy of tumors. One of skill in the art can readily identify such viruses, and can adapt them for the methods described herein. Viruses used in the methods described herein also can be further modified to improve the suitability of the virus for use as a therapeutic and/or diagnostic virus.

Oncolytic viruses include viruses that preferentially infect and accumulate in tumor cells and viruses that are modified to do so. Viruses and viral vectors include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

Typically, the virus for use in the methods is a cytoplasmic virus which does not require entry of viral nucleic acid molecules in to the nucleus of the host cell during the viral life cycle. A variety of cytoplasmic viruses are known, including, but not limited to, poxviruses, African swine flu family viruses, and various RNA viruses such as picornaviruses, caliciviruses, togaviruses, coronaviruses and rhabdoviruses. Exemplary cytoplasmic viruses provided herein are viruses of the poxvirus family, including orthopoxviruses. Exemplary of poxviruses are vaccinia viruses.

Viruses for use in the methods provided herein typically are modified viruses, which are modified relative to the wild-type virus. Such modifications of the viruses provided can enhance one or more characteristics of the virus. Such characteristics can include, but are not limited to, attenuated pathogenicity, reduced toxicity, preferential accumulation in tumor, increased ability to activate an immune response against tumor cells, increased immunogenicity, increased or decreased replication competence, and ability to express additional exogenous proteins, and combinations thereof. For examples, the viruses can be modified to express one or more detectable gene products, including proteins that can be used for detecting, imaging and monitoring of infected tumor cells. In other examples, the viruses can be modified to express one or more gene products for the therapy of a tumor.

Viruses for use in the methods provided herein can contain one or more heterologous nucleic acid molecules inserted into the genome of the virus. A heterologous nucleic acid molecule can contain an open reading frame operatively linked to a promoter for expression or can be a non-coding sequence that alters the attenuation of the virus. In some cases, the heterologous nucleic acid replaces all or a portion of a viral gene.

a. Poxviruses

In some examples, the virus for use in the methods provided herein is selected from the poxvirus family. Poxviruses include Chordopoxyiridae such as orthopoxvirus, parapoxvirus, avipoxvirus, capripoxvirus, leporipoxvirus, suipoxvirus, molluscipoxvirus and yatapoxvirus, as well as Entomopoxyirinae such as entomopoxvirus A, entomopoxvirus B, and entomopoxvirus C. One skilled in the art can select a particular genera or individual chordopoxyiridae according to the known properties of the genera or individual virus, and according to the selected characteristics of the virus (e.g., pathogenicity, ability to elicit an immune response, preferential tumor localization, preferential tumor cell infection), the intended use of the virus, the tumor type and the host organism. Exemplary chordopoxyiridae genera are orthopoxvirus and avipoxvirus.

Avipoxviruses are known to infect a variety of different birds and have been administered to humans. Exemplary avipoxviruses include canarypox, fowlpox, juncopox, mynahpox, pigeonpox, psittacinepox, quailpox, peacockpox, penguinpox, sparrowpox, starlingpox, and turkeypox viruses.

Orthopoxviruses are known to infect a variety of different mammals including rodents, domesticated animals, primates and humans. Several orthopoxviruses have a broad host range, while others have narrower host range. Exemplary orthopoxviruses include buffalopox, camelpox, cowpox, ectromelia, monkeypox, raccoon pox, skunk pox, tatera pox, uasin gishu, vaccinia, variola, and volepox viruses. In some embodiments, the orthopoxvirus selected can be an orthopoxvirus known to infect humans, such as cowpox, monkeypox, vaccinia, or variola virus. Optionally, the orthopoxvirus known to infect humans can be selected from the group of orthopoxviruses with a broad host range, such as cowpox, monkeypox, or vaccinia virus.

i. Vaccinia Viruses

One exemplary orthopoxvirus for use in the methods provided herein is vaccinia virus. Vaccinia virus strains have been shown to specifically colonize solid tumors, while not infecting other organs (see, e.g., Zhang et al. (2007) Cancer Res 67:10038-10046; Yu et al., (2004) Nat Biotech 22:313-320; Heo et al., (2011) Mol Ther 19:1170-1179; Liu et al. (2008) Mol Ther 16:1637-1642; Park et al., (2008) Lancet Oncol, 9:533-542). Vaccinia is a cytoplasmic virus, thus, it does not insert its genome into the host genome during its life cycle. The linear dsDNA viral genome of vaccinia virus is approximately 200 kb in size, encoding a total of approximately 200 potential genes. A variety of vaccinia virus strains are available for uses in the methods provided, including Western Reserve (WR) (SEQ ID NO: 18), Copenhagen (SEQ ID NO: 19), Tashkent, Tian Tan, Lister, Wyeth, IHD-J, and IHD-W, Brighton, Ankara, MVA, Dairen I, LIPV, LC 16M8, LC16MO, LIVP, WR 65-16, Connaught, New York City Board of Health.

Exemplary vaccinia viruses are Lister or LIVP vaccinia viruses. In one embodiment, the Lister strain can be an attenuated Lister strain, such as the LIVP (Lister virus from the Institute of Viral Preparations, Moscow, Russia) strain, which was produced by further attenuation of the Lister strain. The LIVP strain was used for vaccination throughout the world, particularly in India and Russia, and is widely available. In another embodiment, the viruses and methods provided herein can be based on modifications to the Lister strain of vaccinia virus.

Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression. LIVP and its production are described, for example, in U.S. Pat. Nos. 7,588,767, 7,588,771, 7,662,398 and 7,754,221 and U.S. Patent Publication Nos. US 2007/0202572, US 2007/0212727, US 2010/0062016, US 2009/0098529, US 2009/0053244, US 2009/0155287, US 2009/0117034, US 2010/0233078, US 2009/0162288, US 2010/0196325, US 2009/0136917 and US 2011/0064650.

Vaccinia virus possesses a variety of features for use in cancer gene therapy and vaccination including broad host and cell type range, a large carrying capacity for foreign genes (up to 25 kb of exogenous DNA fragments (approximately 12% of the vaccinia genome size) can be inserted into the vaccinia genome), high sequence homology among different strains for designing and generating modified viruses in other strains, and techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). A variety of vaccinia virus strains are available, including Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary of vaccinia viruses for use in the methods provided herein include, but are not limited to, Lister strain or LIVP strain of vaccinia viruses.

The exemplary modifications of the Lister strain described herein (see Example 1) also can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health). The modifications of the Lister strain described herein also can be adapted to other viruses, including, but not limited to, viruses of the poxvirus family, adenoviruses, herpes viruses and retroviruses.

LIVP strains that can be used in the methods provided herein include LIVP clonal strains derived from LIVP that have a genome that is or is derived from or is related to a the parental sequence set forth in SEQ ID NO: 2 (see U.S. Patent Application Ser. No. 61/517,297, which is incorporated herein by reference). These include LIVP clonal strains that have been shown to exhibit greater anti-tumorigenicity and/or reduced toxicity compared to the recombinant or modified virus strain designated GLV-1h68 (having a genome set forth in SEQ ID NO:1; and U.S. Patent Application Ser. No. 61/517,297). In particular, the clonal strains are present in a virus preparation propagated from LIVP. Exemplary LIVP clonal strains include but are not limited to LIVP 1.1.1 (SEQ ID NO: 20), LIVP 2.1.1 (SEQ ID NO: 21), LIVP 4.1.1 (SEQ ID NO: 22), LIVP 5.1.1 (SEQ ID NO: 23), LIVP 6.1.1 (SEQ ID NO: 24), LIVP 7.1.1 (SEQ ID NO: 25), and LIVP 8.1.1 (SEQ ID NO: 26).

For purposes herein, the methods are exemplified with GLV-1h68, but it is understood that the methods can be employed with any oncolytic virus that can be administered for therapy and/or diagnosis of a tumor.

The LIVP and clonal strains for use in the methods provided herein have a sequence of nucleotides that have at least 70%, such as at least 75%, 80%, 85% or 90% sequence identity to SEQ ID NO: 2. For example, the clonal strains have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, and 100% identical SEQ ID NO: 2. Such LIVP clonal viruses include viruses that differ in one or more open reading frames (ORF) compared to the parental LIVP strain that has a sequence of amino acids set forth in SEQ ID NO: 2. The LIVP clonal virus strains can contain a nucleotide deletion or mutation in any one or more nucleotides in any ORF compared to SEQ ID NO: 2, or can contain an addition or insertion of viral DNA compared to SEQ ID NO: 2.

In some examples, the LIVP strain for use in the methods is a clonal strain of LIVP or a modified form thereof containing a sequence of nucleotides selected from: nucleotides 2,256-181,114 of SEQ ID NO:20, nucleotides 11,243-182,721 of SEQ ID NO:21, nucleotides 6,264-181,390 of SEQ ID NO:22, nucleotides 7,044-181,820 of SEQ ID NO:23, nucleotides 6,674-181,409 of SEQ ID NO:24, nucleotides 6,716-181,367 of SEQ ID NO:25 or nucleotides 6,899-181,870 of SEQ ID NO:26.

In some examples, the LIVP strain for use in the methods is a clonal strain of LIVP or a modified form thereof containing a sequence of nucleotides that has at least 97% sequence identity to a sequence of nucleotides 2,256-181,114 of SEQ ID NO:20, nucleotides 11,243-182,721 of SEQ ID NO:21, nucleotides 6,264-181,390 of SEQ ID NO:22, nucleotides 7,044-181,820 of SEQ ID NO:23, nucleotides 6,674-181,409 of SEQ ID NO:24, nucleotides 6,716-181,367 of SEQ ID NO:25 or nucleotides 6,899-181,870 of SEQ ID NO:26.

(1) Modified Vaccinia Viruses

Exemplary vaccinia viruses for use in the methods provided include vaccinia viruses with insertions, mutations or deletions. Exemplary insertions, mutations or deletions include those that result in an attenuated vaccinia virus relative to the wild type strain. For example, vaccinia virus insertions, mutations or deletions can decrease pathogenicity of the vaccinia virus, for example, by reducing the toxicity, reducing the infectivity, reducing the ability to replicate, or reducing the number of non-tumor organs or tissues to which the vaccinia virus can accumulate. Other exemplary insertions, mutations or deletions include, but are not limited to, those that increase antigenicity of the virus, those that permit detection, monitoring, or imaging, those that alter attenuation of the virus, and those that alter infectivity. For example, the ability of vaccinia viruses provided herein to infect and replicate within tumors can be enhanced by mutations that increase the extracellular enveloped form of the virus (EEV) that is released from the host cell, as described elsewhere herein. Modifications can be made, for example, in genes that are involved in nucleotide metabolism, host interactions and virus formation or at other nonessential gene loci. Any of a variety of insertions, mutations or deletions of the vaccinia virus known in the art can be used herein, including insertions, mutations or deletions of: the thymidine kinase (TK) gene, the hemagglutinin (HA) gene, and F14.5L gene, among others (e.g., A35R, E2L/E3L, K1L/K2L, superoxide dismutase locus, 7.5K, C7-K1L, J2R, B13R+B14R, A56R, A26L or 14L gene loci). The vaccinia viruses for use in the methods provided herein also can contain two or more insertions, mutations or deletions. Thus, included are vaccinia viruses containing two or more insertions, mutations or deletions of the loci provided herein or other loci known in the art. The viruses can be based on modifications to the Lister strain and/or LIVP strain of vaccinia virus. Any known vaccinia virus, or modifications thereof that correspond to those provided herein or known to those of skill in the art to reduce toxicity of a vaccinia virus. Generally, however, the mutation will be a multiple mutant and the virus will be further selected to reduce toxicity.

The modified viruses for use in the methods provided herein can encode heterologous gene products. The heterologous nucleic acid is typically operably linked to a promoter for expression of the heterologous gene in the infected cells. Suitable promoter include viral promoters, such as a vaccinia virus natural and synthetic promoters. Exemplary vaccinia viral promoters include, but are not limited to, P11k, P7.5 k early/late, P7.5 k early, P28 late, synthetic early P_{SE}, synthetic early/late P_{SEL} and synthetic late P_{SL} promoters.

(2) Exemplary Modified Vaccinia Viruses

Exemplary vaccinia viruses include those derived from vaccinia virus strain GLV-1h68 (also designated RVGL21 and for clinical trial as GL-ONC1; see SEQ ID NO:1), which has been described in U.S. Pat. Pub. No. 2005-0031643, now U.S. Pat. No. 7,588,767; see, also U.S. provisional application Ser. No. 61/517,297, which provides sequences of clonal strains of LIVP and derivatives thereof, including GLV-1h68).

GLV-1h68 contains DNA insertions into gene in an LIVP strain of vaccinia virus (SEQ ID NO: 2). The LIVP vaccinia virus strain was originally prepared by adapting the Lister strain (ATCC Catalog No. VR-1549) to calf skin (Institute of Viral Preparations, Moscow, Russia, Al'tshtein et al., (1983) Dokl. Akad. Nauk USSR 285:696-699)). It is available from the Institute of Viral Preparations. GLV-1h68 contains expression cassettes encoding detectable marker proteins in the F14.5L (also designated in LIVP as F3), thymidine kinase (TK) and hemagglutinin (HA) gene loci. An expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) is inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) is inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)guSA) is inserted into the HA gene locus. The GLV-1h68 virus exhibits a strong preference for accumulation in tumor tissues compared to non-tumorous tissues following systemic administration of the virus to tumor bearing subjects. This preference is significantly higher than the tumor selective accumulation of other vaccinia viral strains, such as WR (see, e.g. U.S. Pat. Pub. No. 2005-0031643 and Zhang et al. (2007) Cancer Res. 67(20):10038-10046).

Modified viruses for use in the methods provided herein include the strain designed GLV-1h68 (SEQ ID NO: 1) and all strains, derivatives, and modified forms thereof that contain different or additional insertions, deletions, and also variants thereof (see, e.g., U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650). Exemplary viruses are generated by replacement of one or more expression cassettes of the GLV-1h68 strain with heterologous DNA encoding gene products for therapy and/or imaging.

Non-limiting examples of viruses that are derived from attenuated LIVP viruses, such as GLV-1h68, and that can be employed in the methods provided, include, but are not limited to, LIVP viruses described in U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325 and 2009/0136917, which are incorporated herein by reference in their entirety. For example, the vaccinia virus can be selected from among GLV-1h22, GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, or GLV-1h86, which are described in U.S. Patent Publication No. 2009/0098529 and GLV-1h104, GLV-1h105, GLV-1h106, GLV-1h107, GLV-1h108 and GLV-1h109, which are described in U.S. Patent Publication No. 2009/0053244; GLV-1h99, GLV-1h100, GLV-1h101, GLV-1h139, GLV-1h146, GLV-1h151, GLV-1h152 and GLV-1h153, which are described in U.S. Patent Publication No. 2009/0117034.

Exemplary of viruses which have one or more expression cassettes removed from GLV-1h68 and replaced with a heterologous non-coding DNA molecule include GLV-1h70, GLV-1h71, GLV-1h72, GLV-1h73, GLV-1h74, GLV-1h85, and GLV-1h86. GLV-1h70 contains (P_{SEL})Ruc-GFP inserted into the F14.5L gene locus, (P_{SEL})rTrfR and (P_{7.5k})LacZ inserted into the TK gene locus, and a non-coding DNA molecule inserted into the HA gene locus in place of (P₁₁ₖ)gusA. GLV-1h71 contains a non-coding DNA molecule inserted into the F14.5L gene locus in place of (P_{SEL})Ruc-GFP, (P_{SEL})rTrfR and (P_{7.5k})LacZ inserted into the TK gene locus, and (P₁₁ₖ)gusA inserted into the HA gene locus. GLV-1h72 contains (P_{SEL})Ruc-GFP inserted into the F14.5L gene locus, a non-coding DNA molecule inserted into the TK gene locus in place of (P_{SEL})rTrfR and (P_{7.5k})LacZ, and P₁₁ₖgusA inserted into the HA gene locus. GLV-1h73 contains a non-coding DNA molecule inserted into the F14.5L gene locus in place of (P_{SEL})Ruc-GFP, (P_{SEL})rTrfR and (P_{7.5k})LacZ inserted into the TK gene locus, and a non-coding DNA molecule inserted into the HA gene locus in place of (P₁₁ₖ)gusA. GLV-1h74 contains a non-coding DNA molecule inserted into the F14.5L gene locus in place of (P_{SEL})Ruc-GFP, a non-coding DNA molecule inserted into the TK gene locus in place of (P_{SEL)}rTrfR and (P_{7.5k})LacZ, and a non-coding DNA molecule inserted into the HA gene locus in place of (P₁₁ₖ)gusA. GLV-1h85 contains a non-coding DNA molecule inserted into the F14.5L gene locus in place of (P_{SEL})Ruc-GFP, a non-coding DNA molecule inserted into the TK gene locus in place of (P_{SEL})rTrfR and (P_{7.5k})LacZ, and (P₁₁ₖ)gusA inserted into the HA gene locus. GLV-1h86 contains (P_{SEL})Ruc-GFP inserted into the F14.5L gene locus, a non-coding DNA molecule inserted into the TK gene locus in place of (P_{SEL})rTrfR and (P_{7.5k})LacZ, and a non-coding DNA molecule inserted into the HA gene locus in place of (P₁₁ₖ)gusA.

Other exemplary viruses include, but are not limited to, LIVP viruses that encode additional imaging agents such as ferritin and/or a transferrin receptor (e.g., GLV-1h82 and GLV-1h83 which encode E. coli ferritin at the HA locus; GLV-1h82 addition encodes the human transferrin receptor at the TK locus) or a click beetle luciferase-red fluorescent protein fusion protein (e.g., GLV-1h84, which encodes CBG99 and mRFP1 at the TK locus). During translation, the two proteins are cleaved into two individual proteins at picornavirus 2A element (Osborn et al., Mol. Ther. 12: 569-74, 2005). CBG99 produces a more stable luminescent signal than does Renilla luciferase with a half-life of greater than 30 minutes, which makes in vitro and in vivo assays more convenient. mRFP1 provides improvements in in vivo imaging relative to GFP since mRFP1 can penetrate tissue deeper than GFP. Other exemplary viruses include, but are not limited to, LIVP viruses that encode the far-red fluorescent protein TurboFP635 (scientific name “Katushka”) from the sea anemone Entacmaea quadricolor, such as for example, GLV-1h188 (SEQ ID NO: 3), GLV-1h189 (SEQ ID NO: 4), GLV-1h190 (SEQ ID NO: 5), GLV-1h253 (SEQ ID NO: 6), and GLV-1h254 (SEQ ID NO: 7).

Other exemplary viruses include, but are not limited to, LIVP viruses that express one or more therapeutic gene products, such as angiogenesis inhibitors (e.g., GLV-1h81, which contains DNA encoding the plasminogen K5 domain (SEQ ID NO: 14) under the control of the vaccinia synthetic early-late promoter in place of the gusA expression cassette at the HA locus in GLV-1h68; GLV-1h104, GLV-1h105 and GLV-1h106, which contain DNA encoding a truncated human tissue factor fused to the αᵥβ₃-integrin RGD binding motif (tTF-RGD) (SEQ ID NO: 15) under the control of a vaccinia synthetic early promoter, vaccinia synthetic early/late promoter or vaccinia synthetic late promoter, respectively, in place of the LacZ/rTFr expression cassette at the TK locus of GLV-1h68; GLV-1h107, GLV-1h108 and GLV-1h109, which contain DNA encoding an anti-VEGF single chain antibody G6 (SEQ ID NO: 13) under the control of a vaccinia synthetic early promoter, vaccinia synthetic early/late promoter or vaccinia synthetic late promoter, respectively, in place of the LacZ/rTFr expression cassette at the TK locus of GLV-1h68) and proteins for tumor growth suppression (e.g., GLV-1h90, GLV-1h91 and GLV-1h92, which express a fusion protein containing an IL-6 fused to an IL-6 receptor (sIL-6R/IL-6) (SEQ ID NO: 17) under the control of a vaccinia synthetic early promoter, vaccinia synthetic early/late promoter or vaccinia synthetic late promoter, respectively, in place of the gusA expression cassette at the HA locus in GLV-1h68; and GLV-1h96, GLV-1h97 and GLV-1h98, which express IL-24 (melanoma differentiation gene, mda-7; SEQ ID NO: 16) under the control of a vaccinia synthetic early promoter, vaccinia synthetic early/late promoter or vaccinia synthetic late promoter, respectively, in place of the Ruc-GFP fusion gene expression cassette at the F14.5L locus of GLV-1h68). Additional therapeutic gene products that can be engineered in the viruses provided herein also are described elsewhere herein.

Exemplary transporter proteins that can be encoded by the viruses for in vivo imaging and therapy provided herein include, for example, the human norepinephrine transporter (hNET; SEQ ID NO: 27) and the human sodium iodide symporter (hNIS; SEQ ID NO: 28). Exemplary viruses that can be employed in the methods and use provided herein that encode the human norepinephrine transporter (hNET) include, but are not limited to, GLV-1h99, GLV-1h100, GLV-1h101, GLV-1h139, GLV-1h146, and GLV-1h150. GLV-1h99 encodes hNET under the control of a vaccinia synthetic early promoter in place of the Ruc-GFP fusion gene expression cassette at the F14.5L locus of GLV-1h68. GLV-1h100, GLV-1h101 encode hNET under the control of a vaccinia synthetic early promoter or vaccinia synthetic late promoter, respectively, in place of the LacZ/rTFr expression cassette at the TK locus of GLV-1h68. GLV-1h139 encodes hNET under the control of a vaccinia synthetic early promoter in place of the gusA expression cassette at the HA locus in GLV-1h68. GLV-1h146 and GLV-1h150, encode hNET under the control of a vaccinia synthetic early promoter or vaccinia synthetic late promoter, respectively, in place of the LacZ/rTFr expression cassette at the TK locus of GLV-1h100 and GLV-101, respectively. Thus, GLV-1h146 and GLV-1h150 encode hNET and IL-24. Exemplary viruses that can be employed in the methods and use provided herein that encode the human sodium iodide transporter (hNIS) include, but are not limited to, GLV-1h151, GLV-1h152 and GLV-1h153. GLV-1h151, GLV-1h152 and GLV-1h153 encode hNIS under the control of a vaccinia synthetic early promoter, vaccinia synthetic early/late promoter or vaccinia synthetic late promoter, respectively, in place of the gusA expression cassette at the HA locus in GLV-1h68.

b. Other Oncolytic Viruses

Oncolytic viruses for use in the methods provided here are well known to one skill in the art and include, for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175; herpes simplex virus, see, e.g., U.S. Pat. Nos. 7,897,146, 7731,952, 7,550,296, 7,537,924, 6,723,316, 6,428,968 and U.S. Pat. Pub. Nos. 2011/0177032, 2011/0158948, 2010/0092515, 2009/0274728, 2009/0285860, 2009/0215147, 2009/0010889, 2007/0110720, 2006/0039894 and 20040009604; retroviruses, see, e.g., U.S. Pat. Nos. 6,689,871, 6,635,472, 6,639,139, 5,851,529, 5,716,826, 5,716,613 and U.S. Pat. Pub. No. 20110212530; and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

Also included are other therapeutic vaccinia viruses, such as the virus designated JX-594, which is a vaccinia virus that expresses GM-CSF described, for example, in U.S. Pat. No. 6,093,700, and the Wyeth strain vaccinia virus designated JX-594, which is a TK-deleted vaccinia virus that expresses GM-CSF (see, International PCT application No WO 2004/014314, U.S. Pat. No. 5,364,773; Mastrangelo et al. (1998) Cancer Gene Therapy 6:409-422; Kim et al. (2006) Molecular Therapeutics 14:361-370).

In addition, adenoviruses, such as the ONYX viruses and others, have been modified, such as be deletion of EA1 genes, so that they selectively replicate in cancerous cells, and, thus, are oncolytic. Adenoviruses also have been engineered to have modified tropism for tumor therapy and also as gene therapy vectors.

c. Production and Preparation of Virus

The viruses for use in the methods provided herein can be formed by standard methodologies well known in the art for producing and/or modifying viruses. Briefly, the methods can include introducing into viruses one or more genetic modifications, followed by screening the viruses for properties reflective of the modification or for other desired properties.

i. Methods for Generating Recombinant Virus

Standard techniques in molecular biology can be used to generate the modified viruses for use in the methods provided herein. Methods for the generation, of recombinant viruses using recombinant DNA methods are well known in the art (e.g., see U.S. Pat. Nos. 4,769,330, 4,603,112, 4,722,848, 4,215,051, 5,110,587, 5,174,993, 5,922,576, 6,319,703, 5,719,054, 6,429,001, 6,589,531, 6,573,090, 6,800,288, 7,045,313, He et al. (1998) Proc. Natl. Acad. Sci. USA 95(5): 2509-2514, Racaniello et al. (1981) Science 214: 916-919, Hruby et al. (1990) Clin Micro Rev. 3:153-170). Such methods include, but are not limited to, various nucleic acid manipulation techniques, nucleic acid transfer protocols, nucleic acid amplification protocols, and other molecular biology techniques known in the art. For example, point mutations can be introduced into a gene of interest through the use of oligonucleotide mediated site-directed mutagenesis. Alternatively, homologous recombination can be used to introduce a mutation or exogenous sequence into a target sequence of interest. In an alternative mutagenesis protocol, point mutations in a particular gene also can be selected for using a positive selection pressure. See, e.g., Current Techniques in Molecular Biology, (Ed. Ausubel, et al.). Nucleic acid amplification protocols include but are not limited to the polymerase chain reaction (PCR). Use of nucleic acid tools such as plasmids, vectors, promoters and other regulating sequences, are well known in the art for a large variety of viruses and cellular organisms. Nucleic acid transfer protocols include calcium chloride transformation/transfection, electroporation, liposome mediated nucleic acid transfer, N-[1-(2,3-Dioloyloxy)propyl]-N,N,N-trimethylammonium methylsulfate meditated transformation, and others. Further a large variety of nucleic acid tools are available from many different sources including ATCC, and various commercial sources. One skilled in the art will be readily able to select the appropriate tools and methods for genetic modifications of any particular virus according to the knowledge in the art and design choice.

Any of a variety of modifications can be readily accomplished using standard molecular biological methods known in the art. The modifications will typically be one or more truncations, deletions, mutations or insertions of the viral genome. In one example, the modification can be specifically directed to a particular sequence. The modifications can be directed to any of a variety of regions of the viral genome, including, but not limited to, a regulatory sequence, to a gene-encoding sequence, or to a sequence without a known role. Any of a variety of regions of viral genomes that are available for modification are readily known in the art for many viruses, including the viruses specifically listed herein. As a non-limiting example, the loci of a variety of vaccinia genes provided herein and elsewhere exemplify the number of different regions that can be targeted for modification in the viruses provided herein. In some examples, the modification can be fully or partially random, whereupon selection of any particular modified virus can be determined according to the desired properties of the modified the virus. These methods include, for example, in vitro recombination techniques, synthetic methods and in vivo recombination methods as described, for example, in Sambrook et al. Molecular Cloning: A Laboratory Manual, 2nd edition, Cold Spring Harbor Laboratory Press, cold Spring Harbor N.Y. (1989), and in the Examples disclosed herein.

The viruses for use in the methods provided herein can be modified to express an exogenous gene. Exemplary exogenous gene products include proteins and RNA molecules. The modified viruses can express an additional detectable gene product, a therapeutic gene product, a gene product for manufacturing or harvesting, or an antigenic gene product for antibody harvesting. In some examples, the virus can encode a reporter protein, such as, for example, a fluorescent protein, a luminescent protein, a receptor or an enzyme. The characteristics of such gene products are described herein and elsewhere. In some examples of modifying an organism to express an exogenous gene, the modification also can contain one or more regulatory sequences to regulate expression of the exogenous gene. As is known in the art, regulatory sequences can permit constitutive expression of the exogenous gene or can permit inducible expression of the exogenous gene. Further, the regulatory sequence can permit control of the level of expression of the exogenous gene. In some examples, inducible expression can be under the control of cellular or other factors present in a tumor cell or present in a virus-infected tumor cell. In other examples, inducible expression can be under the control of an administrable substance, including IPTG, RU486 or other known induction compounds. Any of a variety of regulatory sequences are available to one skilled in the art and can be selected according to known factors and design preferences. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

In other examples, organ or tissue-specific expression can be controlled by regulatory sequences. In order to achieve expression only in the target organ, for example, a tumor, the foreign nucleotide sequence can be linked to a tissue specific promoter and used for gene therapy. Such promoters are well known to those skilled in the art (see e.g., Zimmermann et al. (1994) Neuron 12: 11-24; Vidal et al. (1990) EMBO J. 9: 833-840; Mayford et al. (1995) Cell 81: 891-904; and Pinkert et al. (1987) Genes & Dev. 1: 268-76).

In some examples, the viruses can be modified to express two or more proteins, where any combination of the two or more proteins can be one or more detectable gene products, therapeutic gene products, gene products for manufacturing or harvesting or antigenic gene products for antibody harvesting. In one example, a virus can be modified to express a detectable protein and a therapeutic protein. In another example, a virus can be modified to express two or more gene products for detection or two or more therapeutic gene products. For example, one or more proteins involved in biosynthesis of a luciferase substrate can be expressed along with luciferase. When two or more exogenous genes are introduced, the genes can be regulated under the same or different regulatory sequences, and the genes can be inserted in the same or different regions of the viral genome, in a single or a plurality of genetic manipulation steps. In some examples, one gene, such as a gene encoding a detectable gene product, can be under the control of a constitutive promoter, while a second gene, such as a gene encoding a therapeutic gene product, can be under the control of an inducible promoter. Methods for inserting two or more genes in to a virus are known in the art and can be readily performed for a wide variety of viruses using a wide variety of exogenous genes, regulatory sequences, and/or other nucleic acid sequences.

Methods of producing recombinant viruses are known in the art (Falkner F G & Moss B (1990) Transient dominant selection of recombinant vaccinia viruses. J Virol 64(6):3108-3111). Provided herein for exemplary purposes are methods of producing a recombinant vaccinia virus. A recombinant vaccinia virus with an insertion in the F14.5L gene (NotI site of LIVP) can be prepared by the following steps: (a) generating (i) a vaccinia shuttle plasmid containing the modified F14.5L gene inserted at restriction site X and (ii) a dephosphorylated wt VV (VGL) DNA digested at restriction site X; (b) transfecting host cells infected with PUV-inactivated helper VV (VGL) with a mixture of the constructs of (i) and (ii) of step a; and (c) isolating the recombinant vaccinia viruses from the transfectants. One skilled in the art knows how to perform such methods, for example by following the instructions given in co-pending U.S. application Ser. Nos. 10/872,156 and 11/238,025; see also Timiryasova et al. (2001) Biotechniques 31: 534-540. In one example, restriction site X is a unique restriction site.

A variety of suitable host cells also are known to the person skilled in the art and include many mammalian, avian and insect cells and tissues which are susceptible for vaccinia virus infection, including chicken embryo, rabbit, hamster and monkey kidney cells, for example, HeLa cells, RK13, CV-1, Vero, BSC40 and BSC-1 monkey kidney cells.

2. Expression of Therapeutic and Reporter Gene Products

The oncolytic viruses used in the methods provided herein can be modified to express one or more heterologous genes. Gene expression can include expression of a protein encoded by a gene and/or expression of an RNA molecule encoded by a gene. The viruses can be modified express one or more genes for the therapy of a tumor. The viruses also can be modified express one or more genes whose products are detectable or whose products can provide a detectable signal. These genes are often called “reporter genes”, and their products are called “reporter proteins” or “reporter gene products”. A reporter gene and its product are generally amenable to assays that are sensitive, quantitative, rapid, easy and reproducible. Many reporter genes have been described in the art, and their detection can be effected in a variety of ways. These heterologous genes can be introduced into the viruses and used to easily assess, for example, the activity of the promoter under which the reporter gene is controlled, the level of transcription and/or translation of the virally encoded genes, and in some instances, by inference, certain activities of the host cell in which the virus resides. In some examples, the reporter protein interacts with host cell proteins, resulting in a detectable change in the properties of the reporter protein.

Expression of heterologous genes can be controlled by a constitutive promoter, or by an inducible promoter. Expression also can be influenced by one or more proteins or RNA molecules expressed by the virus. Host cell factors also can influence the expression of heterologous genes. Depending upon the factors that influence the expression, the level of expression of a reporter gene can be used as an indicator for various processes within the virus, or within the host cell in which the virus grows. For example, if expression of the reporter gene relies on viral factors produced only after viral DNA replication occurs, then the level of the expression of the reporter gene can be used as a measure of the level of viral DNA replication.

a. Exemplary Reporter Gene Products

A variety of reporter genes that encode detectable proteins are known in the art, and can be expressed in the viruses in the methods provided herein. Detectable proteins include receptors or other proteins that can specifically bind a detectable compound, proteins that can emit a detectable signal such as a fluorescence signal, and enzymes that can catalyze a detectable reaction or catalyze formation of a detectable product. Thus, reporter proteins can be assayed by detecting endogenous characteristics, such as enzymatic activity or spectrophotometric characteristics, or indirectly with, for example, antibody-based assays.

i. Fluorescent Proteins

In some examples, the oncolytic viruses for use in the methods provided can express a gene encoding a protein that is a fluorescent protein. Fluorescent proteins emit fluorescence by absorbing and re-radiating the energy of light. Fluorescence can yield relatively high levels of light, compared to, for example, chemiluminescence, and is readily detected by various means known in the art and described herein. Many fluorescent proteins are known in the art and have been widely used as reporter proteins. The first cloned of these, and the most well-known, is green fluorescent protein (GFP) from the Aequorea victoria (Prasher et al. (1987) Gene 111: 229-233), which is a 27 kDa protein that produces a green fluorescence emission with a peak wavelength at 507 nm following excitation at either 395 or 475 nm. GFP also has been cloned from Aequorea coerulescens (Gurskaya et al. (2003) Biochem J. 373:403-8). The wild-type GFP gene has been modified by, for example, point mutation, optimizing codon usage or introducing a Kozak translation initiation site, to generate multiple variants with improved and/or alternate properties. For example, a variant termed enhanced green fluorescent protein (EGFP) contains a single point mutation that shifts the excitation wavelength to 488 nm, which is in the cyan region, and optimized codon usage which yields greater expression in mammalian systems (Yang et al. (1996) Nucl Acids Res. 24 4592-4593). Other variants are spectral variants which display blue, cyan and yellowish-green fluorescent emissions, generally referred to as blue fluorescent protein (BFP), cyan fluorescent protein (CFP), and yellow fluorescent protein (YFP). Examples of these and other variants of GFP include, but are not limited to, those described in U.S. Pat. Nos. 5,625,048, 5,804,387, 6,027,881, 6,150,176, 6,265,548, and 6,608,189.

GFP-like proteins have been isolated from other organisms, particularly the reef corals in the class Anthazoa. While some of the GFP-like proteins emit a green fluorescence, such as the green fluorescent protein from the anthozoan coelenterates Renilla reniformis and Renilla kollikeri (sea pansies) (U.S. Pat. Pub. No. 2003/0013849), others fluoresce with an even wider range of colors than the GFP variants, including blue, green, yellow, orange, red and purple (see e.g., U.S. Pat. No. 7,166,444, Miyawaki et al. (2002) Cell Struct Func 27: 343-347, Labas et al. (2002) Proc. Natl. Acad. Sci. USA 99:4256-4261). Examples of the GFP-like fluorescent proteins include, but are not limited to, those set forth in Table 3.

TABLE 3
Examples of GFP-like proteins

| | | Excitation | Emission | |
|---|---|---|---|---|
| Protein ID | | maxima | maxima | |
| (alternate ID) | Species | (nm) | (nm) | Color |
| amajGFP (amFP486) | Anemonia majano | 458 | 486 | green |
| dsfrGFP (DsFP483) | Discosoma striata | 456 | 484 | green |
| clavGFP (CFP484) | Clavularia sp. | 443 | 483 | green |
| cgigGFP | Condylactis gigantea | 399, 482 | 496 | green |
| hcriGFP | Heteractis crispa | 405, 481 | 500 | green |
| ptilGFP | Ptilosarcus sp. | 500 | 508 | green |
| rmueGFP | Renilla muelleri | 498 | 510 | green |
| zoanGFP (zFP560) | Zoanthus sp. | 496 | 506 | green |
| asulGFP (asFP499) | Anemonia sulcata | 403, 480 | 499 | green |
| dis3GFP | Discosoma sp.3 | 503 | 512 | green |
| dendGFP | Dendronephthya sp. | 494 | 508 | green |
| mcavGFP | Montastraea cavernosa | 506 | 516 | green |
| rfloGFP | Ricordea florida | 508 | 518 | green |
| scubGFP1 | Scolymia cubensis | 497 | 506 | green |
| scubGFP2 | Scolymia cubensis | 497 | 506 | green |
| zoanYFP | Zoanthus sp. | 494, 528 | 538 | yellow |
| DsRed (drFP583) | Discosoma sp.1 | 558 | 583 | orange-red |
| dis2RFP (dsFP593) | Discosoma sp.2 | 573 | 593 | orange-red |
| zoan2RFP | Zoanthus sp.2 | 552 | 576 | orange-red |
| cpFP611 | Entacmaea quadricolor | 559 | 611 | orange-red |
| mcavRFP | Montastraea cavernosa | 508, 572 | 520, 580 | orange-red |
| rfloRFP | Ricordea florida | 506, 566 | 517, 574 | orange-red |
| Kaede | Trachyphillia geoffroyi | 508, 572 | 518, 582 | orange-red |
| asulCP (asCP) | Anemonia sulcata | 568 | none | purple-blue |
| hcriCP (hcCP) | Heteracis crispa | 578 | none | purple-blue |
| cgigCP (cpCP) | Condylactis gigantea | 571 | none | purple-blue |
| cpasCP (cpCP) | Condylactis parsiflora | 571 | none | purple-blue |
| gtenCP (gtCP) | Goniopora tenuidens | 580 | none | purple-blue |

*Adapted from Miyawaki et al. (2002) Cell Struct Funct 27, 343-34.

Exemplary GFP variants and variants of GFP-like proteins from variety of species are known and can be employed for expression by an oncolytic virus for use in the methods provided herein. Such fluorescent protein include monomeric, dimeric and tetrameric fluorescent proteins. Exemplary monomeric fluorescent proteins include, but are not limited to: violet fluorescent proteins, such as for example, Sirius; blue fluorescent proteins, such as for example, Azurite, EBFP, SBFP2, EBFP2, TagBFP; cyan fluorescent proteins, such as for example, mTurquoise, eCFP, Cerulean, SCFP, TagCFP, mTFP1; green fluorescent proteins, such as for example, GFP, mUkG1, aAG1, AcGFP1, TagGFP2, EGFP, mWasabi, EmGFP (Emerald); yellow fluorescent proteins, such as for example; TagYFP, EYFP, Topaz, SYFP2, YPet, Venus, Citrine; orange fluorescent proteins, such as for example, mKO, mKO2, mOrange, mOrange2, red fluorescent proteins, such as for example; TagRFP, TagRFPt, mStrawberry, mRuby, mCherry; far red fluorescent proteins, such as for example; mRasberry, mKate2, mPlum, and mNeptune; and fluorescent proteins having an increased stokes shift (i.e. >100 nm distance between excitation and emission spectra), such as for example, Sapphire, T-Sapphire, mAmetrine, and mKeima. Exemplary dimeric and tetrameric fluorescent proteins include, but are not limited to: AmCyan1, Midori-Ishi Cyan, copGFP (ppluGFP2), TurboGFP. ZsGreen, TurboYFP, ZsYellow1, TurboRFP, dTomato, DsRed2, DsRed-Express, DsRed-Express2, DsRed-Max, AsRed2, TurboFP602, RFP611, Katushka (TurboFP635), Katushka2, and AQ143. Excitation and emission spectra for exemplary fluorescent proteins are well-known in the art (see also e.g. Chudakov et al. (2010) Physiol Rev 90, 1102-1163).

In particular examples, a GFP or GFP-like protein is selected for expression by an oncolytic virus for use in the methods provided herein. In other particular examples, a red or far-red fluorescent protein is selected for expression by an oncolytic virus for use in the methods provided herein. In further particular examples, the fluorescent protein Katushka (TurboFP635) protein is selected for expression by an oncolytic virus for use in the methods provided herein.

Selection of a particular fluorescent protein depends on variety of factors including, but not limited to, brightness, maturation rate, photostability, aggregation and pH stability of the fluorescent protein (see e.g. Chudakov et al. (2010) Physiol Rev 90, 1102-1163). Typically, a fluorescent protein for expression by an oncolytic virus is selected to provide a detectable signal within a reasonable time following infection of a tumor cell.

Other proteinaceous fluorophores include phycobiliproteins from certain cyanobacteria and eukaryotic algae. These proteins are among the most highly fluorescent known (Oi et al. (1982) J. Cell Biol. 93:981-986), and systems have been developed that are able to detect the fluorescence emitted from as little as one phycobiliprotein molecule (Peck et al. (1989) Proc. Natl. Acad. Sci. USA 86 4087-4091). Phycobiliproteins are classified on the basis of their color into two large groups, the phycoerythrins (red) and the phycocyanins (blue). Examples of fluorescent phycobiliproteins include, but are not limited to, R-Phycoerythrin (R-PE), B-Phycoerythrin (B-PE), Y-Phycoerythrin (Y-PE), C-Phycocyanin (P-PC), R-Phycocyanin (R-PC), Phycoerythrin 566 (PE 566), Phycoerythrocyanin (PEC) and Allophycocyanin (APC). The genes encoding the phycobiliproteins have been cloned from a multitude of species and have been used to express the fluorescent proteins in a heterologous host (Tooley et al. (2001) Proc. Natl. Acad. Sci. USA 98:10560-10565). The genes required for the expression of these or any other fluorophores can be cloned into the viruses used in the methods provided herein to generate a virus with a fluorescent reporter protein.

ii. Bioluminescent Proteins

In some examples, the oncolytic viruses can express a gene encoding a protein that is a bioluminescent protein. Chemiluminescence is a process in which photons are produced when molecules in an excited state transition to a lower energy level in an exothermic chemical reaction. The chemical reactions required to generate the excited states in this process generally proceed at a relatively low rate compared to, for example, fluorescence, and so yield a relatively low rate of photon emission. Because the photons are not required to create the excited states, they do not constitute an inherent background when measuring photon efflux, which permits precise measurement of very small changes in light. Bioluminescence is a form of chemiluminescence that has developed through evolution in a range of organisms, and is based on the interaction of the enzyme luciferase with a luminescent substrate luciferin. The luciferases can produce light of varying colors. For example, the luciferases from click beetles can produce light with emission peaks in the range of 547 to 593 nm, spanning four colors (Wood et al. (1989) Science 244: 700-702).

Thus, luciferases for use in the methods provided are enzymes or photoproteins that catalyze a bioluminescent reaction (i.e., a reaction that produces bioluminescence). Some exemplary luciferases, such as firefly, Gaussia and Renilla luciferases, are enzymes which act catalytically and are unchanged during the bioluminescence generating reaction. Other exemplary luciferases, such as the aequorin photoprotein to which luciferin is non-covalently bound, are changed, such as by release of the luciferin, during bioluminescence-generating reaction. The luciferase can be a protein, or a mixture of proteins (e.g., bacterial luciferase). The protein or proteins can be native, or wild luciferases, or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

Exemplary genes encoding bioluminescent proteins include, but are not limited to, bacterial luciferase genes from Vibrio harveyi (Belas et al. (1982) Science 218: 791-793), and Vibrio fischerii (Foran and Brown, (1988) Nucleic acids Res. 16:177), firefly luciferase (de Wet et al. (1987) Mol. Cell. Biol. 7:725-737), aequorin from Aequorea victoria (Prasher et al. (1987) Biochem. 26:1326-1332), Renilla luciferase from Renilla renformis (Lorenz et al. (1991) Proc. Natl. Acad. Sci. USA 88:4438-4442) and click beetle luciferase from Pyrophorus plagiophthalamus (Wood et al. (1989) Science 244:700-702). Other naturally occurring secreted luciferases include, for example, those from Vargula hilgendorfii, Cypridina noctiluca, Oplophorus gracilirostris, Metridia longa and Gaussia princeps. Native and synthetic forms of the genes can be used in the methods provided herein. The luxA and luxB genes of bacterial luciferase can be fused to produce the fusion gene (Fab2), which can be expressed to produce a fully functional luciferase protein (Escher et al. (1989) Proc. Natl. Acad. Sci. USA 86: 6528-6532). Transformation and expression of these and other genes encoding bioluminescent proteins in viruses can permit detection of viral infection, for example, using a low light and/or fluorescence imaging camera. In some examples, luciferases expressed by viruses can require exogenously added substrates such as decanal or coelenterazine for light emission. In other examples, viruses can express a complete lux operon, which can include proteins that can provide luciferase substrates such as decanal.

Bioluminescence substrates are the compounds that are oxidized in the presence of a luciferase and any necessary activators and which generates light. With respect to luciferases, these substrates are typically referred to as luciferins that undergo oxidation in a bioluminescence reaction. The bioluminescence substrates include any luciferin or analog thereof or any synthetic compound with which a luciferase interacts to generate light. Typical substrates include those that are oxidized in the presence of a luciferase or protein in a light-generating reaction. Bioluminescence substrates, thus, include those compounds that those of skill in the art recognize as luciferins. Luciferins, for example, include firefly luciferin, Cypridina (also known as Vargula) luciferin, coelenterazine, dinoflagellate luciferin, bacterial luciferin, as well as synthetic analogs of these substrates or other compounds that are oxidized in the presence of a luciferase in a reaction the produces bioluminescence.

iii. Other Enzymes

In some examples, the oncolytic viruses can express a gene encoding a protein that can catalyze a detectable reaction. Some commonly used reporter genes encode enzymes or other biochemical markers which, when active in the host cells, cause some visible change in the cells or their environment upon addition of the appropriate substrate. Two examples of this type of reporter are the E. coli genes lacZ (encoding β-galactosidase or “β-gal”) and gusA or iudA (encoding β-glucuronidase or “β-glu”). These bacterial sequences are useful as reporter genes because the cells in which they are expressed, prior to transfection, express extremely low levels (if any) of the enzyme encoded by the reporter gene. When host cells expressing the reporter gene (via heterologous expression from the virus) are incubated with an appropriate substrate, a detectable product is formed. The particular substrate used dictates the type of signal generated and the method of detection required. For example, β-galactosidase substrates include those that, when hydrolyzed by β-galactosidase, form products that can be detected, for example, by spectrophotometry (e.g., o-nitrophenyl-β-D-galactoside (ONPG) or 5-bromo-4-chloro-3-indolyl-β-D-galactopyranoside (X-gal)); fluorometry (e.g., a 4-methyl-umbelliferyl-β-galactopyranoside compound (MUG)); or via chemiluminescence (e.g., 1,2-dioxetane-galactopyranoside derivatives; Bronstein et al. (1996) Clin Chem. 42:1542-1546). Many substrates that facilitate the detection of enzymatic activity by various methods also exist for use with β-glucuronidase, including, but not limited to, 5-bromo-4-chloro-3-indolyl-β-D-glucuronic acid (X-Gluc), which produces a blue precipitate following hydrolysis; p-nitrophenyl β-D-glucuronide which also can be used in a spectrophotometrical format; 4-methylumbelliferyl-β-D-glucuronide (MUG), which can be used in a fluorometric assay; and sodium 3-(4-methoxyspiro{1,2-dioxetane-3,2′-(5′-chloro)-tricyclo[3.3.1.13,7]decan}-4-yl)phenyl-β-D-glucuronate (Glucuron®; U.S. Pat. No. 6,586,196 and Bronstein et al. (1996) Clin Chem. 42:1542-1546), which can be used in a chemiluminescent assay.

Other exemplary reporter genes that can be expressed in the viruses used in the methods provided herein include secreted embryonic alkaline phosphatase (SEAP) and chloramphenicol acetyltransferase (CAT). SEAP is a truncated form of human placental alkaline phosphatase that is secreted into the cell culture supernatant following expression. The alkaline phosphatase activity can be readily assayed using any of the substrates known in the art, and can be visualized by chemiluminescence (e.g., using the substrate CSPD [disodium 3-(4-methoxyspiro[1,2-d]oxetane-3,2′(5′-chloro)-tricyclo[3,3,1,13,7)decan]-4-yl)phenyl phosphate]); fluorescence (e.g., using the substrate MUP [4-methylumbelliferyl phosphate]); or spectrometry (e.g., using the substrate p-nitrophenyl phosphate (PNPP)).

The bacterial gene encoding chloramphenicol acetyltransferase (CAT), which catalyzes the addition of acetyl groups to the antibiotic chloramphenicol also can be cloned into the viruses and used to express a reporter protein. CAT activity can be monitored in several ways. In one method, cells infected by the virus expressing the CAT reporter gene can be lysed and incubated in a reaction mix containing 14C- or 3H-labeled chloramphenicol and n-Butyryl Coenzyme A (n-Butyryl CoA). The expressed heterologous CAT transfers the n-butyryl moiety of the cofactor to chloramphenicol. The reaction products can be extracted, separated and the amount of radioactive n-butyryl chloramphenicol is assayed by liquid scintillation counting. The radioactive n-butyryl chloramphenicol resulting from CAT activity also can be analyzed using thin-layer chromatography.

Additional exemplary reporter genes include, but are not limited to enzymes, such as β-lactamase, alpha-amylase, peroxidase, T4 lysozyme, oxidoreductase and pyrophosphatase.

iv. Proteins that Bind to Detectable Ligands

Exemplary detectable proteins also include proteins that can bind a contrasting agent, chromophore, or a compound or ligand that can be detected. In some examples, the ligand that binds to the detectable protein is covalently attached to a detectable moiety, such, for example a radiolabel, a chromogen, or a fluorescent moiety.

A variety of gene products that can specifically bind a detectable compound are known in the art, including, but not limited to receptors, metal binding proteins (e.g., siderophores, ferritins, transferrin receptors), ligand binding proteins, and antibodies. Any of a variety of detectable compounds can be used, and can be imaged by any of a variety of known imaging methods. Exemplary compounds include receptor ligands and antigens for antibodies. The ligand can be labeled according to the imaging method to be used. Exemplary imaging methods include, but are not limited to, X-rays, magnetic resonance methods, such as magnetic resonance imaging (MRI) and magnetic resonance spectroscopy (MRS), and tomographic methods, including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography and ultrasonic tomography.

Labels appropriate for X-ray imaging are known in the art, and include, for example, Bismuth (III), Gold (III), Lanthanum (III) or Lead (II); a radioactive ion, such as ⁶⁷Copper, ⁶⁷Gallium, ⁶⁸Gallium, ¹¹¹Indium, ¹¹³Indium, ¹²³iodine, ¹²⁵Iodine, ¹³¹Iodine, ¹⁹⁷Mercury, ²⁰³Mercury, ¹⁸⁶Rhenium, ¹⁸⁸Rhedum, ⁹⁷Rubidium, ¹⁰³Rubidium, ⁹⁹Technetium or ⁹⁰Yttrium; a nuclear magnetic spin-resonance isotope, such as Cobalt (II), Copper (II), Chromium (III), Dysprosium (III), Erbium (III), Gadolinium (III), Holmium (III), Iron (II), Iron (III), Manganese (II), Neodymium (III), Nickel (II), Samarium (III), Terbium (III), Vanadium (II) or Ytterbium (III); or rhodamine or fluorescein.

Labels appropriate for magnetic resonance imaging are known in the art, and include, for example, gadolinium chelates and iron oxides. Use of chelates in contrast agents is known in the art. Labels appropriate for tomographic imaging methods are known in the art, and include, for example, β-emitters such as ¹¹C, ¹³N, ¹⁵O or ⁶⁴Cu or γ-emitters such as ¹²³I. Other exemplary radionuclides that can, be used, for example, as tracers for PET include ⁵⁵Co, ⁶⁷Ga, ⁶⁸Ga, ⁶⁰Cu(II), ⁶⁷Cu(II), ⁵⁷Ni, ⁵²Fe and ¹⁸F (e.g., ¹⁸F-fluorodeoxyglucose (FDG)). Examples of useful radionuclide-labeled agents are a ⁶⁴Cu-labeled engineered antibody fragment (Wu et al. (2002) Proc. Natl. Acad. Sci. USA 97: 8495-8500), ⁶⁴Cu-labeled somatostatin (Lewis et al. (1999) J. Med. Chem. 42: 1341-1347), ⁶⁴Cu-pyruvaldehyde-bis(N4-methylthiosemicarbazone)(⁶⁴Cu-PTSM) (Adonai et al. (2002) Proc. Natl. Acad. Sci. USA 99: 3030-3035), ⁵²Fe-citrate (Leenders et al. (1994) J. Neural. Transm. Suppl. 43: 123-132), ⁵²Fe/^{52m}Mn-citrate (Calonder et al. (1999) J. Neurochem. 73: 2047-2055) and ⁵²Fe-labeled iron (III) hydroxide-sucrose complex (Beshara et al. (1999) Br. J. Haematol. 104: 288-295, 296-302).

v. Transporter Proteins

The viruses provided herein also can encode proteins, such as transporter proteins (e.g., the human norepinephrine transporter (hNET) or the human sodium iodide symporter (hNIS)), which can provide increase uptake diagnostic and therapeutic moieties across the cell membrane of infected cells for therapy, imaging or detection (see, e.g. U.S. Patent Pub. No. US-2009-0117034).

vi. Proteins Detectable by Antibodies

Viruses also can be modified to express a heterologous reporter protein that can be detected with antibodies, typically by indirect or direct Enzyme Linked ImmunoSorbent Assay (ELISA). Any protein against which a monoclonal antibody or polyclonal antibodies can be raised can be utilized for these purposes. For example, as a non-radioactive alternative, chloramphenicol acetyltransferase expression can be quantified in an ELISA via immunological detection of the CAT enzyme expressed in the virus (see e.g., Francois et al. (2005) Antimicrob. Agents Chemother. 49:3770-3775). In another example, the well-defined human Growth Hormone (hGH) reporter system can be utilized. When cloned into the viruses and expressed in the infected host cell, the hGH reporter protein can be secreted into the culture medium, which means that cell lysis is not necessary for quantifying the reporter protein. Detection of the secreted hGH can be carried out, for example, using ¹²⁵I-labeled antibodies against the growth hormone or with anti-hGH antibodies bound to the surface of a microtiter plate. For example, the hGH from the supernatant of the culture medium is added to the wells and binds to the antibody on the plate. The bound hGH can be detected in two steps via a digoxigenin-coupled anti-hGH antibody and a peroxidase-coupled anti-digoxigenin antibody. Bound peroxidase can then be quantified by incubation with a substrate.

vii. Fusion Proteins

The viruses also can be modified to express reporter proteins that are fusion proteins, encoded by fusion genes. The fusion protein can contain all or part of an endogenous viral protein, or contain only heterologous amino acids sequences. The fusion protein can contain a polypeptide, protein or fragment thereof that is itself detectable, such as by spectrometry, fluorescence, chemiluminescence, or any other method known in the art, or catalyzes a detectable reaction or some visible change in the host cells or their environment upon addition of the appropriate substrate, or binds a detectable product. In one example, the fusion gene is a fusion of two individual genes that are required for a fully functional dateable product. For example, the luxA and luxB genes of bacterial luciferase can be fused to produce the fusion gene (Fab2), which can be expressed to produce a fully functional luciferase protein, as described above. In another example, the fusion protein can contain more than one detectable element. For example, a fluorescent protein, such as GFP, can be expressed as a fusion protein with a bioluminescent protein, such as luciferase, or another fluorescent protein that differs in the wavelength of light emitted, such as DsRed. In another non-limiting example, an enzyme, such as β-galactosidase, can be expressed as a fusion protein with a protein or polypeptide detectable by antibodies, such as hGH.

viii. Proteins that Interact with Host Cell Proteins

The viruses also can be modified to express a reporter protein that directly interacts with one or more proteins that are expressed in the host cell. This interaction can result in a detectable change in the reporter protein such that the interaction can be measured. If the host cell proteins(s) are expressed during a particular biological process, then the reporter protein can be used to indicate the initiation of this process. In some examples, the reporter protein can be a substrate of a host cell protease. Once cleaved, one or more of the separate cleaved products can be differentially detected over the uncleaved protein. In one example, the virus can be modified to express a protein that contains a caspase target sequence, such as DEVD (SEQ ID NO: 29) or LEVD (SEQ ID NO: 30). For example, an oncolytic virus can be modified to express a fusion protein that contains a caspase target sequence that is flanked by two fluorescent molecules, such as CFP and YFP. Cleavage of the fusion protein results in fluorescent signals that can be differentiated from the uncleaved protein by fluorescence resonance energy transfer (FRET) analysis. FRET is a distance-dependent interaction between the electronic excited states of two dye molecules in which excitation is transferred from a donor molecule to an acceptor molecule without emission of a photon. When two suitable fluorescent molecules are separated by a sufficiently short distance, FRET will occur and observed emission at the wavelength corresponding to the donor will increase. When the molecules are separated further, FRET decreases (Zaccolo et al. (2004) Circ. Res. 94:866-873). The uncleaved fusion protein results in intense FRET, but when caspases are activated in the target cell during apoptosis, the fusion protein is cleaved and the molecules are separated, so FRET diminishes (He et al. (2004) Am. J. Pathol. 164:1901-1913). In other examples, a fusion protein is made of a luciferase and a fluorophore, linked by a cleavage sequence, and cleavage is detected by bioluminescence resonance energy transfer (BRET) analysis (Hu et al. (2005) J. Virol. Methods 128:93-103).

b. Exemplary Therapeutic Gene Products

In some examples, the oncolytic viruses provide oncolytic therapy of a tumor cell without the expression of a therapeutic gene. In some examples, the oncolytic viruses can express one or more genes whose products are useful for tumor therapy. For example, a virus can express a proteins cause cell death or whose products cause an anti-tumor immune response. Such genes can be considered therapeutic genes. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound.

Exemplary therapeutic gene products that can be expressed by oncolytic viruses include, but are not limited to, gene products (i.e., proteins and RNAs), including those useful for tumor therapy, such as, but not limited to, an anticancer agent, an anti-metastatic agent, or an antiangiogenic agent. For example, exemplary proteins useful for tumor therapy include, but are not limited to, tumor suppressors, cytostatic proteins and costimulatory molecules, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor. For example, a large number of therapeutic proteins that can be expressed for tumor treatment in the viruses and methods provided herein are known in the art, including, but not limited to, a transporter, a cell-surface receptor, a cytokine, a chemokine, an apoptotic protein, a mitosis inhibitor protein, an antimitotic oligopeptide, an antiangiogenic factor (e.g., hk5), angiogenesis inhibitors (e.g., plasminogen kringle 5 domain, anti-vascular endothelial growth factor (VEGF) scAb, tTF-RGD, truncated human tissue factor-αᵥβ₃-integrin RGD peptide fusion protein), anticancer antibodies, such as a single-chain antibody (e.g., an antitumor antibody or an antiangiogenic antibody, such as an anti-VEGF antibody or an anti-epidermal growth factor receptor (EGFR) antibody), a toxin, a tumor antigen, a prodrug converting enzyme, a ribozyme, RNAi, and siRNA.

Additional therapeutic gene products that can be expressed by oncolytic viruses include, but are not limited to, cell matrix degradative genes, such as but not limited to, relaxin-1 and MMP9, and genes for tissue regeneration and reprogramming human somatic cells to pluripotency, such as but not limited to, nAG, Oct4, NANOS, Neogenin-1, Ngn3, Pdx1 and Mafa.

Costimulatory molecules for use in the methods provided herein include any molecules which are capable of enhancing immune responses to an antigen/pathogen in vivo and/or in vitro. Costimulatory molecules also encompass any molecules which promote the activation, proliferation, differentiation, maturation or maintenance of lymphocytes and/or other cells whose function is important or essential for immune responses.

An exemplary, non-limiting list of therapeutic proteins includes tumor growth suppressors such as IL-24, WT1, p53, pseudomonas A endotoxin, diphtheria toxin, Arf, Bax, HSV TK, E. coli purine nucleoside phosphorylase, angiostatin and endostatin, p16, Rb, BRCA1, cystic fibrosis transmembrane regulator (CFTR), Factor VIII, low density lipoprotein receptor, beta-galactosidase, alpha-galactosidase, beta-glucocerebrosidase, insulin, parathyroid hormone, alpha-1-antitrypsin, rsCD40L, Fas-ligand, TRAIL, TNF, antibodies, microcin E492, diphtheria toxin, Pseudomonas exotoxin, Escherichia coli Shiga toxin, Escherichia coli Verotoxin 1, and hyperforin. Exemplary cytokines include, but are not limited to, chemokines and classical cytokines, such as the interleukins, including, but not limited to, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factor (GM-CSF), erythropoietin and exemplary chemokines including, but not limited to CXC chemokines such as IL-8 GROα, GROβ, GROγ, ENA-78, LDGF-PBP, GCP-2, PF4, Mig, IP-10, SDF-1α/β, BUNZO/STRC33, I-TAC, BLC/BCA-1; CC chemokines such as MIP-1α, MIP-1β, MDC, TECK, TARC, RANTES, HCC-1, HCC-4, DC-CK1, MIP-3α, MIP-3β, MCP-1, MCP-2, MCP-3, MCP-4, Eotaxin, Eotaxin-2/MPIF-2, I-309, MIP-5/HCC-2, MPIF-1, 6Ckine, CTACK, MEC; lymphotactin; and fractalkine. Exemplary other costimulatory molecules include immunoglobulin superfamily of cytokines, such as B7.1, B7.2.

Exemplary therapeutic proteins that can be expressed by oncolytic viruses used in the methods provided herein include, but are not limited to, erythropoietin (e.g., SEQ ID NO: 12), an anti-VEGF single chain antibody (e.g., SEQ ID NO: 13), a plasminogen K5 domain (e.g., SEQ ID NO: 14), a human tissue factor-αvβ3-integrin RGD fusion protein (e.g., SEQ ID NO: 15), interleukin-24 (e.g., SEQ ID NO: 16), or immune stimulators, such as IL-6-IL-6 receptor fusion protein (e.g., SEQ ID NO: 17).

In some examples, the oncolytic viruses used in the methods provided herein can express one or more therapeutic gene products that are proteins that convert a less active compound into a compound that causes tumor cell death. Exemplary methods of conversion of such a prodrug compound include enzymatic conversion and photolytic conversion. A large variety of protein/compound pairs are known in the art, and include, but are not limited to, Herpes simplex virus thymidine kinase/ganciclovir, Herpes simplex virus thymidine kinase/(E)-5-(2-bromovinyl)-2′-deoxyuridine (BVDU), varicella zoster thymidine kinase/ganciclovir, varicella zoster thymidine kinase/BVDU, varicella zoster thymidine kinase/(E)-5-(2-bromovinyl)-1-beta-D-arabinofuranosyluracil (BVaraU), cytosine deaminase/5-fluorouracil, cytosine deaminase/5-fluorocytosine, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid (CMDA), carboxypeptidase A/methotrexate-phenylamine, cytochrome P450/acetaminophen, cytochrome P450-2B1/cyclophosphamide, cytochrome P450-4B1/2-aminoanthracene, 4-ipomeanol, horseradish peroxidase/indole-3-acetic acid, nitroreductase/CB1954, rabbit carboxylesterase/7-ethyl-10-[4-(1-piperidino)-1-piperidino]carbonyloxy-camptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin glucuronide, thymidine phosphorylase/5′-deoxy-5-fluorouridine, deoxycytidine kinase/cytosine arabinoside, and linamarase/linamarin.

Other therapeutic gene products that can be expressed by the oncolytic viruses used in the methods provided herein include siRNA and microRNA molecules. The siRNA and/or microRNA molecule can be directed against expression of a tumor-promoting gene, such as, but not limited to, an oncogene, growth factor, angiogenesis promoting gene, or a receptor. The siRNA and/or microRNA molecule also can be directed against expression of any gene essential for cell growth, cell replication or cell survival. The siRNA and/or microRNA molecule also can be directed against expression of any gene that stabilizes the cell membrane or otherwise limits the number of tumor cell antigens released from the tumor cell. Design of an siRNA or microRNA can be readily determined according to the selected target of the siRNA; methods of siRNA and microRNA design and down-regulation of genes are known in the art, as exemplified in U.S. Pat. Pub. Nos. 2003-0198627 and 2007-0044164, and Zeng et al., Molecular Cell 9:1327-1333 (2002).

Therapeutic gene products include viral attenuation factors, such as antiviral proteins. Antiviral proteins or peptides can be expressed by the viruses provided herein. Expression of antiviral proteins or peptides can control viral pathogenicity. Exemplary viral attenuation factors include, but are not limited to, virus-specific antibodies, mucins, thrombospondin, and soluble proteins such as cytokines, including, but not limited to TNFα, interferons (for example IFNα, IFNβ, or IFNγ) and interleukins (for example IL-1, IL-12 or IL-18).

Another exemplary therapeutic gene product that can be expressed by the oncolytic viruses used in the methods provided herein is a protein ligand, such as antitumor oligopeptide. Antitumor oligopeptides are short protein peptides with high affinity and specificity to tumors. Such oligopeptides could be enriched and identified using tumor-associated phage libraries (Akita et al. (2006) Cancer Sci. 97(10):1075-1081). These oligopeptides have been shown to enhance chemotherapy (U.S. Pat. No. 4,912,199). The oligopeptides can be expressed by the viruses provided herein. Expression of the oligopeptides can elicit anticancer activities on their own or in combination with other chemotherapeutic agents. An exemplary group of antitumor oligopeptides is antimitotic peptides, including, but not limited to, tubulysin (Khalil et al. (2006) Chembiochem. 7(4):678-683), phomopsin, hemiasterlin, taltobulin (HTI-286, 3), and cryptophycin. Tubulysin is from myxobacteria and can induce depletion of cell microtubules and trigger the apoptotic process. The antimitotic peptides can be expressed by the viruses provide herein and elicit anticancer activities on their own or in combination with other therapeutic modalities.

Another exemplary therapeutic gene product that can be expressed by the oncolytic viruses used in the methods provided herein is a protein that sequesters molecules or nutrients needed for tumor growth. For example, the virus can express one or more proteins that bind iron, transport iron, or store iron, or a combination thereof. Increased iron uptake and/or storage by expression of such proteins not only, increases contrast for visualization and detection of a tumor or tissue in which the virus accumulates, but also depletes iron from the tumor environment. Iron depletion from the tumor environment removes a vital nutrient from the tumors, thereby deregulating iron hemostasis in tumor cells and delaying tumor progression and/or killing the tumor.

Additionally, iron, or other labeled metals, can be administered to a tumor-bearing subject, either alone, or in a conjugated form. An iron conjugate can include, for example, iron conjugated to an imaging moiety or a therapeutic agent. In some cases, the imaging moiety and therapeutic agent are the same, e.g., a radionuclide. Internalization of iron in the tumor, wound, area of inflammation or infection allows the internalization of iron alone, a supplemental imaging moiety, or a therapeutic agent (which can deliver cytotoxicity specifically to tumor cells or deliver the therapeutic agent for treatment of the wound, area of inflammation or infection). These methods can be combined with any of the other methods provided herein.

In some examples, the oncolytic viruses used in the methods provided herein can be modified to express one or more antigens to elicit antibody production against an expressed gene product and enhance the immune response against the infected tumor cell. The sustained release of antigen can result in an immune response by the viral-infected host, in which the host can develop antibodies against the antigen, and/or the host can mount an immune response against cells expressing the antigen, including an immune response against tumor cells. Thus, the sustained release of antigen can result in immunization against tumor cells. In some embodiments, the viral-mediated sustained antigen release-induced immune response against tumor cells can result in complete removal or killing of all tumor cells. The immunizing antigens can be endogenous to the virus, such as vaccinia antigens on a vaccinia virus used to immunize against smallpox, measles, mumps, or the immunizing antigens can be exogenous antigens expressed by the virus, such as influenza or HIV antigens expressed on a viral capsid surface. In the case of smallpox, for example, a tumor specific protein antigen can be carried by an attenuated vaccinia virus (encoded by the viral genome) for a smallpox vaccine. Thus, the viruses provided herein, including the modified vaccinia viruses can be used as vaccines.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005/0031643). Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production.

The administered virus can stimulate humoral and/or cellular immune response in the subject, such as the induction of cytotoxic T lymphocytes responses. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

i. Anti-Metastatic Genes

The oncolytic viruses used in the methods provided herein can encode one more anti-metastatic agents that inhibit one or more steps of the metastatic cascade. In some examples, the viruses provided herein encode one more anti-metastatic agents that inhibit invasion of local tissue. In other examples, the oncolytic viruses used in the methods provided herein encode one more anti-metastatic agents that inhibit intravasation into the bloodstream or lymphatics. In other examples, the oncolytic viruses used in the methods provided herein encode one more anti-metastatic agents that inhibit cell survival and transport through the bloodstream or lymphatics as emboli or potentially single cells. In other examples, the oncolytic viruses used in the methods provided herein encode one more anti-metastatic agents that inhibit cell lodging in microvasculature at the secondary site. In other examples, the oncolytic viruses used in the methods provided herein encode one more anti-metastatic agents that inhibit growth into microscopic lesions and subsequently into overt metastatic lesions. In other examples, the oncolytic viruses used in the methods provided herein encode one more anti-metastatic agents that inhibit metastasis formation and growth within the primary tumor, where the inhibition of metastasis formation is not a consequence of inhibition of primary tumor growth. Anti-metastatic agents can inhibit specific steps in the metastatic cascade or multiple steps in the metastatic cascade.

An anti-metastatic agent expressed by a virus for use in the methods provided herein that inhibits metastasis of a tumor in one cell type can inhibit metastasis of other types of tumor cells. For example, an anti-metastatic agent expressed by a virus for use in the methods provided herein that inhibits metastasis of breast tumors also can inhibit metastasis of melanoma tumors (Welch et al. J. Natl. Cancer Inst. 95(12):839-841 (2003); Welch et al. J. Natl. Cancer Inst. 91:1351-1353 (1999); Kauffman et al. J. Urol. 169:1122-1133 (2003); Shevde et al., Cancer Lett. 198:1-20 (2003)).

Anti-metastatic agents expressed by the viruses provided herein can directly or indirectly inhibit one or more steps of the metastatic cascade. Exemplary anti-metastatic agents that can be expressed by the oncolytic viruses used in the methods provided herein include, but are not limited to, the following: BRMS-1 (Breast Cancer Metastasis Suppressor 1), CRMP-1 (Collapsin Response Mediator Protein-1), CRSP-3 (Cofactor Required for Sp1 transcriptional activation subunit 3), CTGF (Connective Tissue Growth Factor), DRG-1 (Developmentally-regulated GTP-binding protein 1), E-Cad (E-cadherin), gelsolin, KAI1, KiSS1 (Kisspeptin 1/Metastin), kispeptin-10, kispeptin-13, kispeptin-14, kispeptin-54, LKB1 (STK11 (serine/threonine kinase 11)), JNKK1/MKK4 (c-Jun-NH2-Kinase Kinase/Mitogen activated Kinase Kinase 4), MKK6 (mitogen activated kinase kinase 6), MKK7 (mitogen activated kinase kinase 7), Nm23 (NDP Kinase A), RASSF1-8 (Ras association (RalGDS/AF-6) domain family members), RKIP (Raf kinase inhibitor protein), RhoGDI2 (Rho GDP dissociation inhibitor 2), SSECKS (src-suppressed C-kinase substrate), Syk, TIMP-1 (Tissue inhibitor of metalloproteinase-1), TIMP-2 (Tissue inhibitor of metalloproteinase-2), TIMP-3 (Tissue inhibitor of metalloproteinase-3), TIMP-4 (Tissue inhibitor of metalloproteinase-4), TXNIP/VDUP1 (Thioredoxin-interacting protein). Such list of anti-metastatic agents is not meant to be limiting. Any gene product that can suppress metastasis formation via a mechanism that is independent of inhibition of growth within the primary tumor is encompassed by the designation of an anti-metastatic agent or metastasis suppressor and can be expressed by a virus as provided herein. One of skill in the art can identify anti-metastatic genes and can construct a virus expressing one or more anti-metastatic genes for therapy.

Exemplary anti-metastatic agents exist within many different types of cellular compartments and are not limited to any specific type of biomolecule. Anti-metastatic agents that are expressed by the viruses provided herein can localize within a variety of cellular compartments within the infected cell, on the surface of the infected cell and/or secreted by the infected cell. For example, anti-metastatic agents can be cell surface receptors, such as, for example KAI1, E cadherin and CD44; intracellular signaling molecules, such as, for example, MKK4, SSeCKs, Nm23, RhoGDI2, DRG-1, and RKIP; secreted ligands, such as, for example TIMPs and KiSS1, nuclear transcription factors and cofactors, such as, for example BRMS1, TXNIP and CRSP3, and proteins localized to the mitochondria, such as, for example, caspase 8 (Welch et al. J. Natl. Cancer Inst. 95(12):839-841 (2003). Anti-metastatic agents also encompass intracellular signaling molecules including cytoskeletal associated proteins, such as, for example, RhoGDI2 and gelsolin, and cytosolic proteins, such as, for example, JNKK1/MKK4, nm23-H1 and RKIP (see, e.g., Dong et al. Science, 268:884-886 (1995); Yin and Stossel, Nature, 281:583-6 (1979); Shimizu et al. Biochem. Biophys. Res. Commun. 175:199-206 (1991); Boller et al., J Cell Biol. 100:327-332 (1985); Girgrah et al., Neuroreport 2:441-444 (1991); Nash et al., Front Biosci. 11:647-59 (2006); Yeung et al., Nature 401:173-177 (1999); Bosnar et al., Exp. Cell Res. 298:275-284 (2004); Rinker-Schaeffer et al., Clin. Cancer Res. 12:3882-3889 (2006)).

c. Operable Linkage to Promoter

Heterologous nucleic acid sequences encoding a therapeutic or reporter protein can be expressed in the viruses by being operably linked to a promoter. The heterologous nucleic acid can be operatively linked to a native promoter or a heterologous (with respect to the virus) promoter. Any promoter known to initiate transcription of an operably-linked open reading frame can be used. The choice of promoter can, however, affect the timing (in relation to viral infection and replication) and the level of the expression of the reporter gene. In some instances, certain requirements exist when operably linking heterologous nucleic acid to the promoter to ensure optimal expression. For example, when a reporter gene is operably linked to a promoter for expression in vaccinia viruses, the heterologous nucleic acid typically does not contain any intervening sequences, such as introns, as the virus does not splice its transcripts. Methods and parameters for operably linking heterologous nucleic acids sequences to promoters for successful expression are well known in the art (see, e.g., U.S. Pat. Nos. 4,769,330, 4,603,112, 4,722,848, 4,215,051, 5,110,587, 5,174,993, 5,922,576, 6,319,703, 5,719,054, 6,429,001, 6,589,531, 6,573,090, 6,800,288, 7,045,313; He et al. (1998) Proc. Natl. Acad. Sci. USA 95(5): 2509-2514; Racaniello et al. (1981) Science 214: 916-919; Hruby et al. (1990) Clin Micro Rev. 3:153-170).

i. Promoter Characteristics

The heterologous nucleic acid can be operatively linked to a native promoter or a heterologous (with respect to the virus) promoter. Any suitable promoters, including synthetic and naturally-occurring and modified promoters, can be used. The promoter region includes specific sequences that are involved in polymerase recognition, binding and transcription initiation. These sequences can be cis acting or can be responsive to trans acting factors. Promoters, depending upon the nature of the regulation, can be constitutive or regulated. Regulated promoters can be inducible or environmentally responsive (e.g., respond to cues such as pH, anaerobic conditions, osmoticum, temperature, light, or cell density). Inducible promoters can include, but are not limited to, a tetracycline-repressed regulated system, ecdysone-regulated system, and rapamycin-regulated system (Agha-Mohammadi and Lotze (2000) J. Clin. Invest. 105(9): 1177-1183). Many promoter sequences are known in the art. See, for example, U.S. Pat. Nos. 4,980,285; 5,631,150; 5,707,928; 5,759,828; 5,888,783; 5,919,670, and, Sambrook, et al. Molecular Cloning: A Laboratory Manual, 2nd Ed., Cold Spring Harbor Press (1989). Synthetic promoters also can be generated. Specific cis elements that can function to modulate a minimal promoter, such as one that contains only a TATA box and an initiator sequence, can be identified and used to generate a promoter that is optimized for the intended use (Edelman et al. (2000) Proc. Natl. Acad. Sci. USA 97:3038-3043). Synthetic promoters for the expression of proteins in vaccinia virus are known in the art, and can include various regulatory elements that dictate the expression profile of the protein (such as the stage in the viral life cycle at which the protein is expressed), and/or enhance expression (see e.g., Pfleiderer et al. (1995) J Gen Virol. 76:2957-2962, Hammond et al. (1997) J Virol Methods. 66:135-138, Chakrabarti et al. (1997) BioTechniques 23:1094-1097). Synthetic promoters also include chemically synthesized promoters, such as those described in U.S. Pat. Pub. No. 2004/0171573.

Promoters that are responsive to external factors, either directly or indirectly, can be selected for use. External factors can include, for example, drugs and inhibitors, such as chemotherapeutic drugs. In one example, the heterologous nucleic acid, such as that which encodes a reporter protein, is operably linked to a promoter that is sensitive to one or more chemotherapeutic drugs. That is, the expression of the heterologous protein from the promoter is inhibited by the chemotherapeutic agent. In another example, the heterologous nucleic acid, such as that which encodes a reporter protein, is operably linked to a promoter that is resistant to one or more chemotherapeutic drugs. That is, the expression of the heterologous protein from the promoter is unaffected by the chemotherapeutic agent. Such a promoter can be of any origin, including mammalian or viral, and be natural or synthetic.

Promoters also can be selected for use on the basis of the relative expression levels that they initiate. Strong promoters are those that support a relatively high level of expression, while weak promoters are those that support a relatively low level of expression. For example, the vaccinia virus synthetic early/late and late promoters are relatively strong promoters, whereas vaccinia synthetic early, P7.5 k early/late, P7.5 k early, and P28 late promoters are relatively weaker promoters (see e.g., Chakrabarti et al. (1997) BioTechniques 23(6) 1094-1097).

ii. Viral and Host Factors

Expression of heterologous proteins can be influenced by one or more proteins or molecules expressed by the virus, or one or more factors expressed by the host. For example, various viral transcription factors can bind other proteins or to the promoter sequence to initiate transcription, or various host factors can interact with one or more regions in the promoter sequence, or with one or more other factors, to initiate transcription. The expression or availability of these molecules and proteins can dictate, for example, level of expression, or the timing of expression, of the heterologous protein under the control of the promoter with which the factors interact.

In one example, the expression of a heterologous protein, such as a reporter protein, from a virus can be controlled temporally by using a promoter that requires interaction with one or more host or viral factors that are expressed, or are available, at a particular stage of the viral life cycle, to initiate transcription. Vaccinia virus coordinates its progression through its replicative cycle by expressing individual proteins at specific times. The temporal regulation of gene expression is controlled at the level of transcriptional initiation, and occurs through a cascade. The transcription factors required for intermediate genes are expressed as early proteins, factors required for late genes are intermediate gene products and the late genes products are packaged into the virions and act as transcription factors for early genes. For example, the vaccinia virus early transcription factor (ETF), which is a dimer made from the products of two late genes, interacts with two regions of the early promoters and recruits the RNA polymerase to the site of transcription. Initiation of transcription results in the synthesis of the early genes within minutes of viral entry into the cell, and is independent of de novo protein synthesis because ETF and the RNA polymerase are already present in the virion. In some instances, genes are expressed continuously, which can be achieved by a tandem arrangement of early and intermediate or late promoters operably linked to the open reading frame (Broyles et al. (1986) Proc. Natl. Acad. Sci. USA 83:3141-3145, Ahn et al. (1990) Mol Cell Biol. 10:5433-5441).

Nearly all viruses, including, but not limited to, poxviruses (including vaccinia virus), adenoviruses, herpesviruses, flaviviruses and caliciviruses link the switch from early to late gene expression to genome replication. The intermediate genes are expressed immediately post-replication, followed closely thereafter by transcription of the late genes. In the absence of nucleic acid synthesis, transcriptional switch does not occur. Because of this regulated expression, inhibition of genome synthesis by, for example, the addition of inhibitors of nucleic acid synthesis such as cytosine arabinoside (Ara-C), results in the inhibition of intermediate and late gene transcription (Vos et al. (1988) EMBO J. 7:3487-3492, Kao et al. (1987) Virology 159:399-407). Therefore, operably linking a heterologous gene to a viral intermediate or late promoter links its expression in the virally-infected host to certain stages of the viral life cycle i.e., after DNA replication. In contrast, operably linking a heterologous gene to a viral early promoter results in its expression immediately following viral entry into the host cell. By selecting the appropriate promoter, a reporter protein can therefore be used to reflect transcriptional activity at various stages of the viral life cycle, which can be linked to multiple viral and/or host factors, and/or external factors, such as drugs and inhibitors.

iii. Exemplary Promoters

Exemplary promoters include synthetic promoters, including synthetic viral and animal promoters. Native promoters or heterologous promoters include, but are not limited to, viral promoters, such as vaccinia virus and adenovirus promoters. Vaccinia viral promoters can be synthetic or natural promoters, and include vaccinia early, intermediate, early/late and late promoters. Exemplary vaccinia viral promoters for use in the methods can include, but are not limited to, P7.5 k, P11k, PSL, PSEL, PSE, H5R, TK, P28, C11R, G8R, F 17R, I3L, I8R, A1L, A2L, A3L, H1L, H3L, H5L, H6R, H8R, D1R, D4R, D5R, D9R, D11L, D12L, D13L, M1L, N2L, P4b or K1 promoters. Other viral promoters can include, but are not limited to, adenovirus late promoter, Cowpox ATI promoter, T7 promoter, adenovirus late promoter, adenovirus E1A promoter, SV40 promoter, cytomegalovirus (CMV) promoter, thymidine kinase (TK) promoter, or Hydroxymethyl-Glutaryl Coenzyme A (HMG) promoter.

In some examples, it can be desirable to choose promoters that initiate expression at particular time points in the viral life cycle. An exemplary vaccinia early promoter is a synthetic early promoter (PSE), which typically initiates gene expression from 0-3 hours post infection. Exemplary vaccinia late promoters include, but are not limited to, a vaccinia 11k promoter (P11k) and a synthetic late promoter (PSL), which typically initiate gene expression 2-3 hours post-infection. Exemplary promoters in vaccinia virus that are expressed throughout the life cycle include tandem arrangements of vaccinia early and intermediate or late promoters (see e.g., Wittek et al. (1980) Cell 21: 487-493; Broyles and Moss (1986) Proc. Natl. Acad. Sci. USA 83: 3141-3145; Ahn et al. (1990) Mol. Cell. Biol. 10: 5433-54441; Broyles and Pennington (1990) J. Virol. 64: 5376-5382). Exemplary vaccinia early/late promoters that express throughout the vaccinia life cycle include, but are not limited to, a 7.5K promoter (P7.5 k) and a synthetic early/late promoter (PSEL).

In some examples, it can be desirable to choose a promoter of a particular relative strength. For example, in vaccinia, synthetic early/late PSEL and many late promoters (e.g., P11k and PSL) are relatively strong promoters, whereas vaccinia synthetic early, PSE, P7.5 k early/late, P7.5 k early, and P28 late promoters are relatively weak promoters (see e.g., Chakrabarti et al. (1997) BioTechniques 23(6) 1094-1097).

3. Further Modifications of Oncolytic Viruses

The viruses used in the methods provided herein can be further modified. Such modifications can, for example, enhance the ease with which the methods are performed, reduce the time taken to perform the methods, provide conditions of increased safety or suitability for administration, compared to unmodified viruses. Such characteristics can include, but are not limited to, attenuated pathogenicity, reduced toxicity, increased or decreased replication competence, increased, decreased or otherwise altered tropism, increased or decreased sensitivity to drugs, such as nucleoside analogs and any combination thereof. The viruses used in the methods provided herein can be modified by any known method for modifying a virus. For example, the viruses can be modified to express one or more heterologous genes. The heterologous genes can be expressed under the control of endogenous viral promoters, or exogenous (i.e., heterologous to the virus) promoters, including synthetic promoters.

Oncolytic viruses have been genetically altered to attenuate their virulence, to improve their safety profile, enhance their tumor specificity, and they have also been equipped with additional genes, for example cytotoxins, cytokines, prodrug converting enzymes to improve the overall efficacy of the viruses (see, e.g., Kirn et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398 and 7,754,221 and U.S. Pat. Publ. Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650.

The modifications can be effected by any method known in the art, and can be introduced into the virus before, after, simultaneously, or in the absence of, the introduction one or more reporter genes. In certain examples, the virus is modified to attenuate pathogenicity. In some examples, it can be desirable to generate a more attenuated virus. A more attenuated virus can be more suitable for in vivo administration and in in vitro assays, providing a safer environment for laboratory personnel and reducing the laboratory biosafety requirements. Attenuation of the virus can be effected by modification of one or more viral genes, such as by a point mutation, a deletion mutation, an interruption by an insertion, a substitution or a mutation of the viral gene promoter or enhancer regions. In such instances, it is advantageous to first identify a target gene involved in pathogenicity, although random mutagenesis can result in attenuation of the virus. The target genes also are typically non-essential, such that the ability of the virus to propagate without the need of a packaging cell lines is preserved when the genes are not expressed, or expressed at decreased levels. In viruses such as vaccinia virus, mutations in non-essential genes, such as the thymidine kinase (TK) gene or hemagglutinin (HA) gene have been employed to attenuate the virus (e.g., Buller et al. (1985) Nature 317, 813-815, Shida et al. (1988) J. Virol. 62(12):4474-80, Taylor et al. (1991) J. Gen. Virol. 72 (Pt 1):125-30, U.S. Pat. Nos. 5,364,773, 6,265,189, 7,045,313). The inactivation of these genes decreases the overall pathogenicity of the virus without eliminating the ability of the viruses to replicate in certain cell types.

Attenuation also can be effected without eliminating or reducing the expression of one or more particular genes involved in pathogenicity. For example, increasing the number of genes that the virus expresses can cause competition for viral transcription and/or translation factors, which can result in changes in expression of endogenous viral genes. Such changes can affect viral processes involved in viral replication, thus contributing to the attenuation of the virus. For example, viral processes, such as viral nucleic acid replication, transcription of other viral genes, viral mRNA production, viral protein synthesis, or virus particle assembly and maturation, can be affected. Insertion of gene expression cassettes that require binding of host factors for efficient transcription can be used to compete the transcription and/or translation factors away from the endogenous viral promoters and transcripts. For example, insertion of gene expression cassettes that contain vaccinia strong late promoters into vaccinia virus can be used to attenuate expression of endogenous vaccinia late genes.

Viruses provided herein also can contain a modification that alters its infectivity or resistance to neutralizing antibodies. In one non-limiting example deletion of the A35R gene in an vaccinia LIVP strain can decrease the infectivity of the virus. In some examples, the viruses provided herein can be modified to contain a deletion of the A35R gene. Exemplary methods for generating such viruses are described in PCT Publication No. WO2008/100292, which describes vaccinia LIVP viruses GLV-1j87, GLV-1j88 and GLV-1j89, which contain deletion of the A35R gene.

In another non-limiting example, replacement of viral coat proteins (e.g., A34R, which encodes a viral coat glycoprotein) with coat proteins from either more virulent or less virulent virus strains can increase or decrease the clearance of the virus from the subject. In one example, the A34R gene in an vaccinia LIVP strain can be replaced with the A34R gene from vaccinia IHD-J strain. Such replacement can increase the extracellular enveloped virus (EEV) form of vaccinia virus and can increase the resistance of the virus to neutralizing antibodies.

### E. APPLICATIONS OF THE METHOD

1. Assessment and Modification of Treatment and Selection of Viruses and Subjects for Therapy

The methods provided herein for detection of tumor inflammation induced by oncolytic viruses using PFC imaging agents can be employed for a variety of applications including, but not limited to, detection of oncolytic virus localization to a tumor following administration, such as systemic administration to a tumor bearing subject, detection of virus replication in a tumor, identification of subject for therapy with an oncolytic virus, selection of oncolytic viruses for therapy, and monitoring therapy with an oncolytic virus. Because the PFC imaging agents are rapidly taken up by macrophages at the tumor or that are recruited to the tumor, the methods provided herein provided a quick and relatively easy assessment of whether the oncolytic virus has infected the tumor.

In some examples, the level of PFC accumulation in the tumor can be quantified and compared to a control a control or reference sample or database of values corresponding to a known level of tumor inflammation. One of skill in the art can establish objective threshold values for determining whether induction of tumor inflammation as a result if virus infection has occurred. In some examples, infection of the tumor is established by the analyzing the pattern of PFC accumulation (e.g. at the tumor periphery).

As shown in the Examples provided, the accumulation of PFC following virus infection was mainly at the tumor periphery in contrast to the diffuse localization throughout the tumor prior to infection. This distinct change in PFC localization allows one of skill in the art to track the tumor inflammatory response before and after infection and provides a reliable way to confirm whether infection of the tumor has occurred. In addition the strong peripheral localization can be employed to detect and monitor changes in tumor size and morphology.

In practicing the methods, treatment can be modified in accord with the results achieved. For example, if an oncolytic virus is administered to a subject and tumor infection is detected using the methods provided, then the oncolytic therapy can be continued. For example, additional doses of the oncolytic virus can be administered or additional cycles of treatment can be performed. If virus infection is not detected, treatment can be discontinued or modified. For example, an additional dose can be administer at the same dosage or a higher dosage, a different oncolytic virus can be administered, or oncolytic virus can be administered in combination with one or more anticancer agents. In some examples, if virus infection is detected using the methods provided, the subject is selected as a candidate to treatment with the particular oncolytic virus.

As described herein, in some examples, the oncolytic virus is first administered at a dose that is lower than the therapeutic dose in order to assess whether the virus infects the tumor. If the virus infection is detected, then a therapeutic dose of the virus can be administered. Exemplary doses for administration and cycles of treatment are provided elsewhere herein.

In some examples, the methods provided herein are employed to select a particular oncolytic virus treatment. Infection of the tumor is an indicator that the virus will be effective for treatment of the tumor. If virus infection is detected using the methods provided, then the oncolytic virus can be selected as a candidate for therapy of the tumor. In some examples, if virus infection is detected using the methods provided, the oncolytic virus can be selected as a candidate for therapy of a particular type of cancer or a particular type of tumor.

In some examples, the oncolytic virus is administered in combination one or additional anti-cancer agents. Additional exemplary anticancer agents that can be administered for cancer therapy in the methods provided include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. As used herein, reference to an anticancer or chemotherapeutic agent includes combinations or a plurality of anticancer or chemotherapeutic agents unless otherwise indicated. Anticancer agents include anti-metastatic agents.

The methods provided herein can be used in combination with one or more additional methods for detecting or monitoring a cancer or tumor or monitoring an anti-cancer therapy. For example, a tumor or metastasis can be detected by physical examination of subject, laboratory tests, such as blood or urine tests, imaging and genetic testing, such as testing for gene mutations that are known to cause cancer. A tumor or metastasis can be detected using in vivo imaging techniques, such as digital X-ray radiography, mammography, CT (computerized tomography) scanning, MRI (magnetic resonance imaging), ultrasonography and PET (positron emission tomography) scanning. Alternatively, a tumor can be detected using tumor markers in blood, serum or urine, that is, by monitoring substances produced by tumor cells or by other cells in the body in response to cancer. For example, prostate specific antigen (PSA) levels are used to detect prostate cancer in men. Additionally, tumors can be detected and monitored by biopsy.

Any of a variety of monitoring steps can be used to monitor an anti-cancer therapy, including, but not limited to, monitoring tumor size, monitoring anti-(tumor antigen) antibody titer, monitoring anti-virus antibody titer, monitoring the presence and/or size of metastases, monitoring the subject's lymph nodes, monitoring the subject's weight or other health indicators including blood or urine markers, monitoring expression of a detectable gene product, and monitoring titer of the oncolytic reporter virus, in a tumor, tissue or organ of a subject.

2. Application of the Methods to Tumor Therapies with Other Microorganisms

Other microorganisms such and yeast and bacteria also have been shown to preferentially accumulate in tumor tissues and can effect tumor treatment and detection imaging and monitoring of tumors (see, e.g. U.S. Patent Application Pub. Nos. US 2004/0234455, US 2005/0031643, US 2008/0193373, and U.S. Pat. No. 7,514,089). Introduction of such microorganisms also induces a potent inflammatory response at the tumor (Patyar et al. (2010) Journal of Biomedical Science 17:21). Previous studies shown that the inflammatory response in infection models, such as in models of Staphylococcus aureus infection, can be visualized using ¹⁹F imaging of intravenously administered PFCs (Hertlein et al. (2010) PLoS ONE 6(3):e18246).

Accordingly, the methods provided herein can be employed for the detection of inflammation induced anti-tumor therapies with other microorganisms in addition to the oncolytic virus therapies described herein. The methods provided herein for administration of PFC imaging agents in combination with oncolytic viruses can be modified accordingly and applied to methods of administration of PFC imaging agents in combination with other microorganisms, such as bacteria. Methods for administration of microorganisms for tumor diagnosis and treatment are known in the art and can be combined with the methods herein for detection of tumor inflammation induced by the microorganism. Exemplary microorganisms for tumor therapy include bacteria, particularly attenuated or non-pathogenic bacteria, including a mutual, commensal or probiotic strain of bacteria or an attenuated pathogenic bacterium. Strains of bacteria include, but are not limited to, bacteria selected from among Escherichia coli, Bacteroides, Eubacterium, Streptococcus, Actinomyces, Veillonella, Nesseria, Prevotella, Campylobacter, Fusobacterium, Eikenella, Porphyromonas, Priopionibacteria, Clostridia, Salmonella, Shigella, Bifidobacteria and Staphylococcus species. Exemplary E. coli bacteria include the probiotic E. coli bacterium, Nissle, such as Nissle 1917.

3. Methods of Tracking Inflammation Using Cells Labeled with PFC Imaging Agents

In some examples, the recruitment of immune cells to active sites of inflammation can be detected and monitored by administered to the tumor-bearing subject cells that are labeled ex vivo with the PFC imaging agent and then administered to the subject. Methods of labeling selected cell populations, such as dendritic cells, monocytes, macrophages and antigen specific T cells, with PFC imaging agents, including PFC emulsions, are known in the art. Exemplary methods are described, for example, in U.S. Patent Pub. App. Nos. US 2008/0292554, US 2007/0253910, US 2011/0110863, and US 2009/0263329; Waiczes et al. (2011) PLoS ONE 6(7):e21981, Ahrens et al. (2005) Nat Biotechnol 23(8):983-987, Noth et al. (1997) Artif Cell Blood Sub 25(3):243-254, and Srinivas et al. (2009) Magn. Reson. Med. 58:725-734). In some examples, the PFC imaging agents are taken up by the cells by co-culturing the cells with the PFC emulsion. In some examples, the PFC contains one or more agents that promote the uptake of the PFC emulsion by the cells, such as, for example, cationic molecules, such as, for example, cationic lipids, cationic polymers, protamine sulfate and polyethyleneimine (PEI). In some examples, the PFC imaging agents are taken up by the cells using electroporation or magnetoelectroporation. Studies have shown that cell electroporation and magnetoelectroporation are effective for MRI in cell culture, including uptake by dendritic cells (Walczak P. (2005) Magn Reson Med. 54(4):769-74) and U.S. provisional application No. 60/792,003). After labeling, cells can be immediately administered, stored, further cultured, enriched, segregated, processed in way that is not incompatible with the intended use of the cells.

In exemplary methods, the immune cells are contacted with the PFC imaging agent ex vivo and then administered to the subject. In some examples, the cells are administered to the blood stream, such as by intravenous administration. The migration of the cells and recruitment to the tumor can then be monitored and imaged by ¹⁹F MRI as described herein. The cells for use in the method can be cells obtained from a donor, such as the subject, or can be cultured or engineered cells for administration. The cells can be administered prior to, at same time as, or following infection of the subject with the oncolytic virus. In some examples, the cells are administered and the tumor is imaged prior to infection with the oncolytic virus and then imaged following virus infection. In some examples, the tumor is imaged at multiple time points following virus infection. The methods provided herein for administration of PFCs emulsions in combination with oncolytic viruses, including parameters for administration and dosages for virus administration and detection and imaging the accumulation of PFCs by ¹⁹F magnetic resonance methods, can be modified accordingly and applied to methods of administration of PFC labeled cells in combination with the virus.

In some examples, the amount of PFC per cell can be quantified prior to administration to the subject. Following administration that amount of signal at the tumor can be quantified and correlated with the amount of cells recruited to a tumor. Such methods provide a measure of the degree of tumor inflammation. Exemplary methods for quantification of cells labeled with PFC imaging agent prior to and following administration to a subject are known in the art and include, for example, methods described in U.S. Patent Pub. No. US 2009/0074673.

4. Therapy of Tumors Using Ultrasonic Release of Therapeutic Agents

Perfluorocarbon imaging agents for use in the methods provided herein also can be employed for the therapy of tumors. (U.S. Patent Application No. 2010/0178305) The nanoemulsions provided herein can contain one or more therapeutic agents for delivery to tumor. In some examples, the PFC nanoemulsion containing one or more therapeutic agents is delivered to a subject having a tumor, wherein the one or more therapeutic agents is released from the nanoemulsion at the site of the tumor. In some examples, the one or more therapeutic agents is released at the site of the tumor using ultrasonic radiation. In some examples, the PFC nanoemulsion contains a therapeutic agent that is an anti-cancer agents, such as a chemotherapeutic agent.

### F. COMBINATIONS, KITS, AND ARTICLES OF MANUFACTURE

Oncolytic viruses, perfluorocarbon (PFC) imaging agents and reagents, materials and devices for detecting accumulation of PFCs or expression of reporter gene encoded by the virus in vivo or ex vivo and combinations thereof, can be provided as combinations of the agents, which optionally can be packaged as kits. In non-limiting examples, an oncolytic virus can be provided in combination with a PFC imaging agent. In other non-limiting examples, an oncolytic virus and/or PFC imaging agent can be provided in combination with reagents for additional analysis of tumor tissue, such as for example, reagent to measure one or more additional tumor cell markers or immune cell markers. For example, a kit can include reagents to fix, permeabilize, stain, or lyse tumor cells or tissues, reagents for amplification of nucleic acid, antibodies for immunohistochemical analysis and/or primers for RT-PCR or qPCR.

Kits can optionally include one or more components such as instructions for use, additional reagents such as diluents, culture media, substrates, antibodies and ligands, and material components, such as sample collection devices, microscope slides, tubes, microtiter plates (e.g., multi-well plate) and containers for practice of the methods. Those of skill in the art will recognize many other possible containers and plates that can be used for contacting the various materials.

Exemplary kits can include reagents used in detection of expression of a reporter gene by the virus. Such reagents can include one or more substrates for detection of a reporter enzyme. In some examples, the kit includes a device, such as a fluorometer, luminometer, or spectrophotometer for assay detection.

In some examples, the oncolytic viruses can be supplied in a lyophilized form, and the kit can optionally include one or more solutions for reconstitution of the virus. In a further example, the lyophilized viruses can be supplied in the kit in appropriate amounts in the wells of one or more microtiter plates or sample tubes.

In some examples, a kit can contain instructions. Instructions typically include a tangible expression describing the virus and, optionally, other components included in the kit, and methods for assay, including methods for preparing and administering the virus, methods for administration and detection of PFCs, methods for preparing tumor samples, or methods for detection of reporter proteins expressed by the viruses.

The articles of manufacture provided herein can contain the viruses and/or PFC imaging agents and packaging materials. Packaging materials for use in packaging products are known to those of skill in the art. See, e.g., U.S. Pat. Nos. 5,323,907, 5,052,558 and 5,033,252. Examples of packaging materials include, but are not limited to, blister packs, bottles, tubes, bags, vials, containers, and any packaging material suitable for a selected formulation and intended use. Articles of manufacture include a label with instructions for use of the packaged material.

One of skill in the art will appreciate the various components that can be included in a kit, consistent with the methods and systems disclosed herein.

### G. EXAMPLES

The following examples are included for illustrative purposes only and are not intended to limit the scope of the claims or disclosure herein.

### Example 1

### Imaging Tumor Colonization with Vaccinia Virus GLV-1h68 in a Melanoma Tumor Model by ¹⁹F MRI

In this example, tumor inflammation induced by intravenous administration of a oncolytic vaccinia virus in a melanoma tumor model was detected and monitored in vivo by administration of a ¹⁹F labeled perfluorocarbon and detection by ¹H/¹⁹F 3D magnetic resonance imaging (MRI). Anatomical spatial ¹⁹F patterns of vaccinia virus treated mice were obtained by overlay of the ¹H anatomical MRI images with the ¹⁹F MRI signal images. Vaccinia virus infected mice were compared to non-infected mice to monitor the effect of vaccinia virus treatment on the recruitment of inflammatory cells to a tumor. Ex vivo ¹⁹F MRI and immunohistochemistry were performed to verify in vivo results. The vaccinia virus GLV-1h68 employed in this study encodes a Renilla luciferase-green fluorescent protein fusion protein (Ruc-GFP), useful for imaging infected tumor cells.

### A. Materials and Methods

Human 1936-MEL melanoma cells from temporally distinct cutaneous metastases were derived as previously described (Sabatino et al. (2008) Cancer Res. 68:222-231). The cells were cultured in RPMI supplemented with 10% FBS, 1 mM HEPES, 1 mM Ciprofloxacin and L-glutamine/penicillin/streptomycin and maintained at 37° C. under 5% CO₂.

1936-MEL cells (7×10⁵ cells) were implanted on day 0 on the right limb of 6-8 week old athymic nude mice (NCI:Hsd:Athymic Nude-Foxn1^{nu}, Harlan) to generate subcutaneous xenograft tumors. Nine (9) days after tumor implantation, 1×10⁷ pfu (in 100 μl) of the attenuated vaccinia virus (VACV) strain GLV-1h68 (see U.S. Pat. No. 7,588,767; SEQ ID NO: 1) was injected intravenously into tumor-bearing animals (n=3). Control animals were injected with the same volume of PBS (n=3). At days 13 and 15 following tumor cell implantation (i.e. 4 and 6 days, respectively, following virus injection or PBS injection), a perfluoro-15-crown-5-ether PFC emulsion (20% v/v, mean droplet diameter 145 nm, 100 μl each injection; V-Sense 1000H, Celsense Inc., Pittsburgh, Pa.) was administered intravenously.

Magnetic resonance imaging (MRI) was performed using a 7 Tesla Bruker Biospec System (Bruker BioSpin GmbH, Reinstetten, Germany) at room temperature small animal scanner and a home-built birdcage coil (inner diameter=40 mm, length=64 mm) adjustable to ¹H and ¹⁹F resonance frequencies. In vivo ¹H 3D turbo-spin-echo (TSE) and ⁹F 3D TSE experiments were performed 17 days after tumor cell implantation (i.e. 8 days following virus injection or PBS injection). Prior to imaging, the animals were anesthetized with isoflurane at 2% over 2-3 hr in a tube-like container. In vivo MRI parameters employed were as follows:

For ¹H 3D TSE imaging: echo time (TE)=30 milliseconds (ms), repetition time (TR)=1000 ms, matrix (MTX)=100×150×125, Resolution (Res)=0.2 mm³, turbo factor (TF)=10, number of averages (NA)=1;

For ¹⁹F 3D TSE imaging: TE=4 ms TR=1000 ms, MTX=40×48×32, Res=0.625 mm³, TF=48, NA=60).

After in vivo MRI, mice were sacrificed and tumors excised. The excised tumors were fixed overnight in 4% paraformaldehyde in PBS and washed twice in PBS. Specimens of approximately 5-15 mm diameter were embedded in 10% Sucrose/PBS with 5% w/v low-melt Agarose. Ex vivo ¹H/¹⁹F 3D TSE was performed on the samples using the following parameters:

For ¹H 3D TSE imaging: TE=81 ms, TR=1000 ms, MTX=300×200×200, Res=0.2 mm³; TF=20, NA=1;

For ¹⁹F 3D TSE imaging: TE=5 ms, TR=1000 ms, MTX=48×64×64, Res=0.625 mm³, TF=64, NA=60.

After ex vivo MRI, the tumors were cut out of the Agarose and dehydrated overnight in 30% w/v Sucrose/PBS. Tumors were embedded for cryosectioning in Tissue-TEK and stored at −80° C.

For immunohistochemistry, the excised tumors were cryo-sectioned into 15 μm slices. Tissue sections were fixed in ice-cold acetone for 10 min. Monocytes and tissue macrophages were labeled with anti-CD68 antibodies (KP-1; Abeam®). Neutrophils were labeled with anti-Ly-6G antibodies (BDBiosciences). For secondary antibody labeling sections were labeled with donkey-anti-rat Cy3 (Jackson ImmunoResearch). The labeled tissue sections were analyzed using stereo-fluorescence microscopy (LeicaMZ16FA). Infection of tumor cells by GLV-1h68 also was visualized by fluorescence imaging of the GFP encoded by GLV-1h68 and expressed by the infected tumor cells.

### B. Results

Both VACV injected and PBS injected animals exhibited ¹⁹F signal located in the tumor by MRI. In PBS injected animals, the ¹⁹F signal was often spread out throughout the entire tumor with low signal intensity. In VACV injected animals, the ¹⁹F signal was located with high signal intensity mainly at the tumor margins. Ex vivo imaging of the VACV injected animals also showed ¹⁹F high signal intensity mainly at the tumor periphery. The distribution of the ¹⁹F MRI signal corresponded well to the strong histological staining pattern of macrophages (CD68) and neutrophils (Ly-6G), which were found mostly at the tumor margins. Detection of GFP expression in the tumor sections confirmed infection of the tumor cells by the VACV.

These results demonstrate that intravenous administration of VACV to tumor-bearing animals results in virus infection of the tumor and induces a massive inflammation characterized by accumulation of CD68-positive macrophages and Ly-6G-positive neutrophils at the tumor periphery. A similar signal pattern was detected by in-vivo and ex vivo ¹⁹F MRI indicating that intravenously administered PFC is taken up by macrophages that are recruited to the tumor, which is significantly enhanced by oncolytic virus infection. Thus, the results indicate that ¹⁹F labeled PFC emulsions can be administered systemically for noninvasive detection of oncolytic virus-induced immune cell recruitment at the tumor.

In PBS-injected controls, accumulation of the PFC in the tumor may be caused by the resident macrophage population in combination with the tumor specific enhanced permeability and retention (EPR) effects (see Fang et al. (2011) Adv. Drug Delivery Reviews 63:136-151). ¹⁹F signal has previously been found in well perfused tumor regions following intravenous injection of PFC emulsions (see Yu et al. (2005) Current Medicinal Chemistry 12:819-848).

Since modifications will be apparent to those of skill in this art, it is intended that this invention be limited only by the scope of the appended claims.

1. A method for detecting or imaging tissues infected by an oncolytic virus, comprising:
administering an oncolytic virus to a subject;
administering an agent for detection of macrophages or inflammatory cells in a subject;
detecting or imaging the accumulation of macrophages or inflammatory cells in a subject, wherein accumulation of the agent in tissues comprising macrophages or inflammatory cells indicates that the oncolytic virus has infected tissues comprising the macrophage or inflammatory cells, thereby detecting or imaging the cells infected by the oncolytic virus.

2. The method of claim 1, wherein the agent detects macrophages.

3. The method of claim 1, wherein:
the subject has a tumor; and
detection of the macrophages or inflammatory cells detects or images cells infected by the oncolytic virus, thereby detecting or imaging tumor cells or tissue in the subject.

4. The method of claim 1, further comprising repeating the method a plurality of times, whereby the progress of oncolytic therapy is monitored by periodically imaging or detecting the macrophages to detect or image changes in the profile of accumulated macrophages and thereby monitor therapy.

5. The method of claim 1, wherein the oncolytic virus does not encode any heterologous proteins for detection of the virus or heterologous proteins that induce a detectable signal.

6. The method of claim 1, wherein the virus is a clonal strain of a virus so that it does not encode any heterologous non-viral gene products.

7. The method of claim 1, wherein the virus encodes a therapeutic protein.

8. The method of claim 7, wherein the therapeutic protein is a protein for treating tumors.

9. The method of claim 4, wherein monitoring assesses whether the therapy is effective by detecting an increase in infected cells within a predetermined time after administration of the virus, wherein the time is sufficient for the oncolytic virus to infect tumor cells.

10. The method of claim 9, wherein the predetermined time is less than 24 days.

11. The method of claim 10, wherein accumulation of the agent in the tumor is indicative that the oncolytic virus treatment is or will be efficacious.

12. The method of claim 1, wherein the agent is detected or imaged in vivo in the subject or is detected ex vivo in a tumor biopsy sample or body fluid sample from the subject.

13. The method of claim 1, wherein the agent is detected or imaged by magnetic resonance imaging (MRI) or magnetic resonance spectroscopy (MRS).

14. The method of claim 1, wherein the imaging agent is administered at a predetermined time prior to the administration of the oncolytic virus or at a predetermined time following the administration of the oncolytic virus.

15. The method of claim 14, wherein the agent is administered 1 hour, 2 hours, 3 hours, 4 hours, 6 hours, 12 hours, 18 hours, 24 hours, 2 days, 3 days, 4 days, 5 days, 6 days, 7 days, 8 days, 9 days, 10 days, 11 days, 12 days, 13 days, 14 days, 3 weeks 4 weeks, 1 month following the administration of the oncolytic virus.

16. The method of claim 1, wherein the agent is administered at the same time as the oncolytic virus or sequentially or intermittently with the virus.

17. The method of claim 1, wherein the agent is detected or imaged at least 1 hour, 2 hours, 4 hours, 6 hours, 12 hours, 18 hours, 24 hours, 30 hours, 36 hours, 42 hours, or 48 hours following the administration of the imaging agent.

18. The method of claim 1, wherein the virus is administered at a dosage for treatment of a tumor or cancer.

19. The method of claim 1, wherein the agent is administered intravenously.

20. The method of claim 1, wherein the oncolytic virus is administered systemically.

21. The method of claim 20, wherein the oncolytic virus is administered by intravenous, intraarterial, intratumoral, endoscopic, intralesional, intramuscular, intraperitoneal, intradermal, intraperitoneal, intravesicular, intraarticular, intrapleural, percutaneous, subcutaneous, oral, parenteral, intranasal, intratracheal, inhalation, intracranial, intraprostatic, intravitreal, topical, ocular, vaginal, or a rectal route of administration.

22. The method of claim 1, wherein the virus is administered a plurality of times and the agent is detected or imaged at a predetermined time after each successive administration of the virus in a cycle of administration.

23. The method of claim 1, wherein the agent for detection of macrophage or inflammatory cells is an imaging agent containing a perfluorocarbon.

24. The method of claim 23, wherein the agent is a perfluorocarbon selected from among a linear perfluorocarbon, branched perfluorocarbon, and cyclic perfluorocarbon.

25. The method of claim 23, wherein agent is a perfluorocarbon imaging agent that contains a perfluorocarbon selected from among a perfluoropolyether, perfluoro crown ether, perfluoroalkane, perfluoropentane, perfluorohexane, perfluorononane, perfluorohexyl bromide, perfluorooctyl bromide, perfluorooctane, perfluorodecalin, perfluorocycloalkane, perfluoro amine, and mixtures thereof.

26. The method of claim 1, wherein the agent is a perfluorocarbon imaging agent that contains two or more perfluorocarbons.

27. The method of claim 1, wherein the agent is a perfluorocarbon imaging agent that is a perfluoroalkyl ether.

28. The method of claim 1, wherein the agent is a perfluorocarbon imaging agent that is perfluoro-15-crown-5-ether.

29. The method of claim 1, wherein the agent is a perfluorocarbon imaging agent that contains a poly(ethylene oxide) block copolymer.

30. The method of claim 29, wherein the poly(ethylene oxide) block copolymer is a poly(ethylene oxide)-polyester block copolymer.

31. The method of claim 30, wherein the poly(ethylene oxide) block copolymer is selected from among poly(ethylene oxide)-block-poly(ε-caprolactone) copolymer, poly(ethylene oxide)-block-(L) polylactide copolymer, poly(ethylene oxide)-block-(D) polylactide copolymer, poly(ethylene oxide)-block-(D,L) polylactide copolymer, and combinations thereof.

32. The method of claim 31, wherein the poly(ethylene oxide) block copolymer is a poly(ethylene oxide)-polyether block copolymer or is a polyethylene-polyether tri-block copolymer or is a poly(ethylene oxide)-polypropylene oxide)-poly(ethylene oxide) (PEO-PPO-PEO) tri-block copolymer.

33. The method of claim 23, wherein the agent is formulated as an emulsion.

34. The method of claim 1, wherein the agent contains a targeting moiety targeting it to macrophage and/or other inflammatory cells and/or a detectable moiety.

35. The method of claim 34, wherein the detectable moiety is a dye or a fluorescent molecule.

36. The method of claim 1, wherein the agent is a perfluorocarbon that comprises an ¹⁸F isotope.

37. The method of claim 1, wherein the subject is a human.

38. The method of claim 1, wherein the subject has a tumor of the lung, breast, colon, brain, prostate, liver, pancreas, esophagus, kidney, stomach, thyroid, bladder, uterus, cervix or ovary.

39. The method of claim 38, wherein the tumor is a metastatic tumor.

40. The method of claim 1, wherein the oncolytic virus is a vaccinia virus.

41. The method of claim 40, wherein the oncolytic virus is a Lister strain virus or a Wyeth strain virus.

42. The method of claim 41, wherein the virus is an LIVP virus.

43. The method of claim 1, wherein the oncolytic virus comprises nucleic acid encoding a heterologous gene product.

44. The method of claim 43, wherein the heterologous nucleic acid encodes a reporter gene product.

45. The method of claim 44, wherein the reporter gene product is a fluorescent protein, a bioluminescent protein, a receptor, or an enzyme.

46. The method of claim 45, wherein the fluorescent protein is selected from among a green fluorescent protein, an enhanced green fluorescent protein, a blue fluorescent protein, a cyan fluorescent protein, a yellow fluorescent protein, a red fluorescent protein, and a far-red fluorescent protein.

47. The method of claim 45, wherein:
the reporter product is a fluorescent protein that is Katushka (TurboFP635), a far-red mutant of the red fluorescent protein from sea anemone Entacmaea quadricolor; or
the reporter product is an enzyme selected from among a luciferase, β-glucuronidase, β-galactosidase, chloramphenicol acetyl tranferase (CAT), alkaline phosphatase, and horseradish peroxidase; or
the reporter product is a receptor that binds to a detectable moiety or a ligand attached to a detectable moiety.

48. The method of claim 47, wherein the reporter is a detectable moiety selected from among a radiolabel, a chromogen and a fluorescent moiety.

49. The method of claim 1, further comprising, in addition to detecting or imaging the macrophage or inflammatory cells, detecting the oncolytic virus by detecting a reporter gene product encoded by the virus.

50. The method of claim 1, wherein the oncolytic virus is detected in vivo in the subject or ex vivo in a tumor biopsy sample from the subject.

51. The method of claim 49, wherein the expression of the reporter gene product is detected by a method selected from among flow cytometry, fluorescence microscopy, fluorescence spectroscopy, magnetic resonance spectroscopy, positron emission tomography and luminescence spectroscopy.

52. The method of claim 1, wherein the virus comprises heterologous nucleic acid that encodes a therapeutic or diagnostic agent.

53. The method of claim 52, wherein:
the heterologous nucleic acid encodes an anticancer agent, an antimetastatic agent, an antiangiogenic agent, an immunomodulatory molecule, an antigen, a cell matrix degradative gene, an enzyme that modifies a substrate to produce a detectable product or signal or is detectable by antibodies, a protein that can bind a contrasting agent; or
the heterologous nucleic acid is a gene for tissue regeneration, reprogramming human somatic cells to pluripotency, optical imaging or detection, PET imaging or MRI imaging.

54. A combination, comprising:
an oncolytic virus; and
an agent for imaging macrophages.

55. The combination of claim 54, wherein the agent is a perfluorocarbon imaging agent.

56. The combination of claim 55, wherein the virus is a vaccinia virus.

57. The method of claim 1, wherein the virus is an LIVP virus designated GLV-1h68 or a derivative thereof.
